SEQUENCE LISTING (43 sequences)

SEQ ID NO: 1 - DNA, 20 bp, Artificial sequence
  Synthetic oligonucleotide
gccaggctggttatgactca

SEQ ID NO: 2 - DNA, 23 bp, Artificial sequence
  Synthetic oligonucleotide
tcagccaggctggttatgactca

SEQ ID NO: 3 - DNA, 16 bp, Artificial sequence
  Synthetic oligonucleotide
gcattctaatagcagc

SEQ ID NO: 4 - DNA, 19 bp, Artificial sequence
  Synthetic oligonucleotide
tcagcattctaatagcagc

SEQ ID NO: 5 - DNA, 16 bp, Artificial sequence
  Synthetic oligonucleotide
aggatatggaaccaaa

SEQ ID NO: 6 - DNA, 19 bp, Artificial sequence
  Synthetic oligonucleotide
tcaaggatatggaaccaaa

SEQ ID NO: 7 - DNA, 16 bp, Artificial sequence
  Synthetic oligonucleotide
gacaacttggagcttg

SEQ ID NO: 8 - DNA, 19 bp, Artificial sequence
  Synthetic oligonucleotide
tcagacaacttggagcttg

SEQ ID NO: 9 - DNA, 16 bp, Artificial sequence
  Synthetic oligonucleotide
ctggtatgaggcctga

SEQ ID NO: 10 - DNA, 19 bp, Artificial sequence
  Synthetic oligonucleotide
tcactggtatgaggcctga

SEQ ID NO: 11 - DNA, 16 bp, Artificial sequence
  Synthetic oligonucleotide
acaataaataccgagg

SEQ ID NO: 12 - DNA, 19 bp, Artificial sequence
  Synthetic oligonucleotide
tcaacaataaataccgagg

SEQ ID NO: 13 - DNA, 14 bp, Artificial sequence
  Synthetic oligonucleotide
  misc_feature (1)..(2): bases at these positions are RNA
  misc_feature (4)..(8): bases at these positions are RNA
  misc_feature (10)..(14): bases at these positions are RNA
cutagcactggccu

SEQ ID NO: 14 - DNA, 20 bp, Artificial sequence
  Synthetic oligonucleotide
agcatagttaacgagctccc

SEQ ID NO: 15 - DNA, 20 bp, Artificial sequence
  Synthetic oligonucleotide
aatggtttattccatggcca

SEQ ID NO: 16 - DNA, 20 bp, Artificial sequence
  Synthetic oligonucleotide
gcagccatggtgatcaggag

SEQ ID NO: 17 - DNA, 17 bp, Artificial sequence
  Synthetic oligonucleotide
ggttcccgaggtgccca

SEQ ID NO: 18 - DNA, 20 bp, Artificial sequence
  Synthetic oligonucleotide
gcacactcagcaggaccccc

SEQ ID NO: 19 - DNA, 20 bp, Artificial sequence
  Synthetic oligonucleotide
ccacctttgggtgaatagca

SEQ ID NO: 20 - DNA, 20 bp, Artificial sequence
  Synthetic oligonucleotide
tgccatttaatgagcttcac

SEQ ID NO: 21 - DNA, 16 bp, Artificial sequence
  Synthetic oligonucleotide
tcccgaatgtccgaca

SEQ ID NO: 22 - DNA, 20 bp, Artificial sequence
  Synthetic oligonucleotide
atcccacgcccctgtccagc

SEQ ID NO: 23 - DNA, 20 bp, Artificial sequence
  Synthetic oligonucleotide
gcagaggtgaagcgaagtgc

SEQ ID NO: 24 - DNA, 20 bp, Artificial sequence
  Synthetic oligonucleotide
ccaatttatgcctacagcct

SEQ ID NO: 25 - DNA, 17 bp, Artificial sequence
  Synthetic oligonucleotide
ggcatagcagcaggatg

SEQ ID NO: 26 - DNA, 20 bp, Artificial sequence
  Synthetic oligonucleotide
aggagttccgcagtatggat

SEQ ID NO: 27 - DNA, 20 bp, Artificial sequence
  Synthetic oligonucleotide
gtgaagcgaagtgcacacgg

SEQ ID NO: 28 - DNA, 20 bp, Artificial sequence
  Synthetic oligonucleotide
gtgcagaggtgaagcgaagt

SEQ ID NO: 29 - DNA, 16 bp, Artificial sequence
  Synthetic oligonucleotide
aggtgaagcgaagtgc

SEQ ID NO: 30 - DNA, 16 bp, Artificial sequence
  Synthetic oligonucleotide
tccgcagtatggatcg

SEQ ID NO: 31 - DNA, 18 bp, Artificial sequence
  Synthetic oligonucleotide
aatttatgcctacagcct

SEQ ID NO: 32 - DNA, 20 bp, Artificial sequence
  Synthetic oligonucleotide
tcttggttacatgaaatccc

SEQ ID NO: 33 - DNA, 20 bp, Artificial sequence
  Synthetic oligonucleotide
cttggttacatgaaatccca

SEQ ID NO: 34 - DNA, 20 bp, Artificial sequence
  Synthetic oligonucleotide
ggaatactcttggttacatg

SEQ ID NO: 35 - DNA, 20 bp, Artificial sequence
  Synthetic oligonucleotide
tggaatactcttggttacat

SEQ ID NO: 36 - DNA, 20 bp, Artificial sequence
  Synthetic oligonucleotide
ttttattgtctctgcctgga

SEQ ID NO: 37 - DNA, 20 bp, Artificial sequence
  Synthetic oligonucleotide
gaatgttttattgtctctgc

SEQ ID NO: 38 - DNA, 20 bp, Artificial sequence
  Synthetic oligonucleotide
aggaatgttttattgtctct

SEQ ID NO: 39 - DNA, 20 bp, Artificial sequence
  Synthetic oligonucleotide
acaggaatgttttattgtct

SEQ ID NO: 40 - DNA, 16 bp, Artificial sequence
  Synthetic oligonucleotide
ggccaatacgccgtca

SEQ ID NO: 41 - DNA, 16 bp, Artificial sequence
  Synthetic oligonucleotide
ggcaattggtgtttaa

SEQ ID NO: 42 - DNA, 20 bp, Artificial sequence
  Synthetic oligonucleotide
attcactttcataatgctgg

SEQ ID NO: 43 - DNA, 18 bp, Artificial sequence
  Synthetic oligonucleotide
tcactttcataatgctgg

(19) United States
(12) Patent Application Publication
(10) Pub. No.: US 2021/0052631 A1
(43) Pub. Date: Feb. 25, 2021

(54) CONJUGATED ANTISENSE COMPOUNDS AND THEIR USE

(71) Applicant: Ionis Pharmaceuticals, Inc., Carlsbad, CA (US)

(72) Inventors: Thazha P. Prakash, Carlsbad, CA (US); Richard Lee, Oceanside, CA (US); Punit P. Seth, Carlsbad, CA (US); Eric E. Swayze, Encinitas, CA (US); Frank Rigo, Carlsbad, CA (US); Michael Oestergaard, Carlsbad, CA (US)

(73) Assignee: Ionis Pharmaceuticals, Inc., Carlsbad, CA (US)

(21) Appl. No.: 15/761,769

(22) PCT Filed: Sep. 26, 2016
(86) PCT No.: PCT/US16/53832

§ 371 Date: Mar. 20, 2018

Related U.S. Application Data
(60) Provisional application No. 62/233,253, filed on Sep. 25, 2015.
(60) Provisional application No. 62/333,101, filed on May 6, 2016.
(60) Provisional application No. 62/399,236, filed on Sep. 23, 2016.

(51) Int. Cl. A61K 31/7125; A61K 9/00

(52) U.S. Cl. CPC A61K 31/7125; A61K 9/0019; C12N 2310/32; C12N 2310/351; C12N 2310/3525; C12N 2310/315; C12N 2310/33

(57) ABSTRACT

Provided herein are oligomeric compounds comprising a modified oligonucleotide and a conjugate group for modulating the amount or activity of a target nucleic acid in extra hepatic tissues and extra hepatic cells. Also provided herein are methods of modulating the amount or activity of an extra-hepatic nucleic acid target in a cell comprising contacting the cell with the oligomeric compound or antisense compound.

### SEQUENCE LISTING

The present application is being filed along with a Sequence Listing in electronic format. The Sequence Listing is provided as a file entitled CORE0136WOSEQ_ST25.txt, created on Sep. 21, 2016, which is 8 KB in size. The information in the electronic format of the sequence listing is incorporated herein by reference in its entirety.

### FIELD

The present embodiments provide oligomeric compounds comprising a modified oligonucleotide and a conjugate group for modulating the amount or activity of a target nucleic acid in extra hepatic tissues and/or extra hepatic cells. Also provided herein are methods of modulating the amount or activity of an extra-hepatic nucleic acid target in a cell comprising contacting the cell with the oligomeric compound or antisense compound.

### BACKGROUND

The principle behind antisense technology is that an antisense compound hybridizes to a target nucleic acid and modulates the amount, activity, and/or function of the target nucleic acid. For example in certain instances, antisense compounds result in altered transcription or translation of a target. Such modulation of expression can be achieved by, for example, target mRNA degradation or occupancy-based inhibition. An example of modulation of RNA target function by degradation is RNase H-based degradation of the target RNA upon hybridization with a DNA-like antisense compound. Another example of modulation of gene expression by target degradation is RNA interference (RNAi). RNAi refers to antisense-mediated gene silencing through a mechanism that utilizes the RNA-induced silencing complex (RISC). An additional example of modulation of RNA target function is by an occupancy-based mechanism such as is employed naturally by microRNA. MicroRNAs are small non-coding RNAs that regulate the expression of protein-coding RNAs. The binding of an antisense compound to a microRNA prevents that microRNA from binding to its messenger RNA targets, and thus interferes with the function of the microRNA. MicroRNA mimics can enhance native microRNA function. Certain antisense compounds alter splicing of pre-mRNA. Regardless of the specific mechanism, sequence-specificity makes antisense compounds attractive as tools for target validation and gene functionalization, as well as therapeutics to selectively modulate the expression of genes involved in the pathogenesis of diseases.

Antisense technology is an effective means for modulating the expression of one or more specific gene products and can therefore prove to be uniquely useful in a number of therapeutic, diagnostic, and research applications. Chemically modified nucleosides may be incorporated into antisense compounds to enhance one or more properties, such as nuclease resistance, pharmacokinetics or affinity for a target nucleic acid. In 1998, the antisense compound, Vitravene® (fomivirsen; developed by Isis Pharmaceuticals Inc., Carlsbad, Calif.) was the first antisense drug to achieve marketing clearance from the U.S. Food and Drug Administration (FDA), and is currently a treatment of cytomegalovirus (CMV)-induced retinitis in AIDS patients. For another example, an antisense oligonucleotide targeting ApoB, KYNAMRO™, has been approved by the U.S. Food and Drug Administration (FDA) as an adjunct treatment to lipid-lowering medications and diet to reduce low density lipoprotein-cholesterol (LDL-C), ApoB, total cholesterol (TC), and non-high density lipoprotein-cholesterol (non HDL-C) in patients with homozygous familial hypercholesterolemia (HoFH).

New chemical modifications have improved the potency and efficacy of antisense compounds, uncovering the potential for oral delivery as well as enhancing subcutaneous administration, decreasing potential for side effects, and leading to improvements in patient convenience. Chemical modifications increasing potency of antisense compounds allow administration of lower doses, which reduces the potential for toxicity, as well as decreasing overall cost of therapy. Modifications increasing the resistance to degradation result in slower clearance from the body, allowing for less frequent dosing. Different types of chemical modifications can be combined in one compound to further optimize the compound's efficacy. Traditionally, antisense compounds, including modified oligonucleotides, have deomonstrated good functional uptake into liver tissue. However, there is still a need to facilitate uptake of antisense compounds into other cell types.

### SUMMARY OF THE INVENTION

After an oligomeric compound is administered to a subject, different organs, tissues, and cells receive different amounts of the oligomeric compound. The distribution of the oligomeric compound to different organs, tissues, and cells depends on many factors. For example, the degree to which a given oligomeric compound binds to plasma proteins may affect the distribution of a given oligomeric compound to various tissues. In certain embodiments, the degree to which a given oligomeric compound is recognized by certain cell-surface receptors may affect the distribution of a given oligomeric compound to various tissues or cells.

Oligomeric compounds typically show good distribution to the liver after administration to a subject. However, in certain embodiments a need exists to deliver oligomeric compounds to other tissues within a subject. For example, a need exists to deliver oligomeric compounds to one or more extra-hepatic tissues such as adipose tissue or muscle tissue. In certain embodiments, the present disclosure provides oligomeric compounds comprising a modified oligonucleotide and a conjugate group, wherein the conjugate group enhances delivery of the modified oligonucleotide to one or more extra-hepatic tissues.

Most oligomeric compounds are metabolized in the liver or kidneys, which can reduce the half life of the oligomeric compound in a subject. For example, in certain embodiments, an oligomeric compound administered to a subject may distribute to the kidneys and then be excreted out in the subject's urine. In another embodiments, Conjugating an oligomeric compound an oligomeric compound administered to a subject may be metabolized in the liver. In certain embodiments, an oligomeric compound administered to a subject is both metabolized by the liver and excreted out through the kidneys. In certain embodiments, the present disclosure provides oligomeric compounds comprising a modified oligonucleotide and a conjugate group, wherein the conjugate group enhances delivery of the modified oligonucleotide. In certain embodiments, the conjugate group enhances delivery of the modified oligonucleotide to a tissue selected from among: skeletal muscle, cardiac muscle, smooth muscle, adipose, white adipose, spleen, bone, intestine, adrenal, testes, ovary, pancreas, pituitary, prostate, skin, uterus, bladder, brain, glomerulus, distal tubular epithelium, breast, lung, heart, kidney, ganglion, frontal cortex, spinal cord, trigeminal ganglia, sciatic nerve, dorsal root ganglion, epididymal fat, diaphragm, and colon.

Oligomeric compounds typically show good uptake in hepatocytes. In certain embodiments, the present disclosure provides oligomeric compounds comprising a modified oligonucleotide and a conjugate group, wherein the conjugate group enhances uptake in a particular cell type. In certain embodiments, the conjugate group enhances uptake in macrophages. In certain embodiments, the conjugate group enhances uptake in cardiomyocytes. In certain embodiments, the conjugate group enhances uptake in fibroblasts. In certain embodiments, the conjugate group enhances uptake in endothelial cells. In certain embodiments, the conjugate group enhances uptake in heart cells.

In certain embodiments, the present disclosure provides oligomeric compounds comprising a modified oligonucleotide and a conjugate group that modulates the amount or activity of a target nucleic acid transcript in an extra-hepatic cell to a greater extent than oligomeric compound comprising unconjugated modified oligonucleotide. In certain embodiments, the present disclosure provides oligomeric compounds comprising a modified oligonucleotide and a conjugate group that modulates the amount or activity of a target nucleic acid transcript in an extra-hepatic tissue to a greater extent than oligomeric compound comprising unconjugated modified oligonucleotide. In certain embodiments, the present disclosure provides oligomeric compounds comprising a modified oligonucleotide and a conjugate group that modulates the amount or activity of a target nucleic acid transcript in an extra-hepatic cell and in an extra-hepatic tissue to a greater extent than oligomeric compound comprising unconjugated modified oligonucleotide.

In certain embodiments, the present disclosure provides oligomeric compounds comprising a modified oligonucleotide and a conjugate group for delivery to extra-hepatic cells. In certain embodiments, the present disclosure provides oligomeric compounds comprising a modified oligonucleotide and a conjugate group for delivery to extra-hepatic tissues. In certain embodiments, the present disclosure provides oligomeric compounds comprising a modified oligonucleotide and a conjugate group wherein the modified oligonucleotide is complementary to a target nucleic acid transcript expressed in one or more extra-hepatic cell types. In certain embodiments, the present disclosure provides oligomeric compounds comprising a modified oligonucleotide and a conjugate group wherein the modified oligonucleotide is complementary to a target nucleic acid transcript expressed in one or more extra-hepatic tissues.

In certain embodiments, the present disclosure provides methods of modulating the amount or activity of a target nucleic acid in an extra-hepatic tissue and/or extra-hepatic cell type. In certain such embodiments, the present disclosure provides methods of treating diseases in which modulating the amount of activity of the target nucleic acid in the liver is not sufficient to provide a therapeutic benefit. The present disclosure provides the following non-limiting embodiments:
Embodiment 1: An oligomeric compound comprising a modified oligonucleotide and a conjugate group wherein:
   the modified oligonucleotide consists of 10-30 linked nucleosides and has a nucleobase sequence complementary to the nucleobase sequence of an extra-hepatic nucleic acid target;
   wherein the conjugate group comprises a conjugate moiety and a conjugate linker,
   wherein the conjugate moiety is selected from among: a lipid, vitamin, steroid, C₅-C₃₀ saturated alkyl group, C₅-C₃₀ unsaturated alkyl group, fatty acid, or lipophilic group; and wherein the conjugate linker comprises at least one cleavable moiety.
Embodiment 2: The oligomeric compound of embodiment 1, wherein the extra-hepatic nucleic acid target is not expressed in the liver at a significant level.
Embodiment 3: The oligomeric compound of embodiment 1, wherein the extra-hepatic nucleic acid target is expressed in the liver at a significant level.
Embodiment 4: The oligomeric compound of any of embodiments 1-3, wherein the extra-hepatic nucleic acid target is expressed in at least one extra-hepatic cell type selected from among: white fat cells, brown fat cells, adipocytes, macrophages, cancer cells, tumor cells, smooth muscle cells, lymphocytes, pulmonary cells, and heart muscle cells.
Embodiment 5: The oligomeric compound of any of embodiments 1-4, wherein the extra-hepatic nucleic acid target is expressed in at least two extra-hepatic cell types.
Embodiment 6: The oligomeric compound of any of embodiments 1-5, wherein the extra-hepatic nucleic acid target is expressed in at least three extra-hepatic cell types.
Embodiment 7: The oligomeric compound of any of embodiments 1-6, wherein the extra-hepatic nucleic acid target is expressed in at least four extra-hepatic cell types.
Embodiment 8: The oligomeric compound of any of embodiments 1-7, wherein the extra-hepatic nucleic acid target is expressed in white fat cells.
Embodiment 9: The oligomeric compound of any of embodiments 1-8, wherein the extra-hepatic nucleic acid target is expressed in brown fat cells
Embodiment 10: The oligomeric compound of any of embodiments 1-9, wherein the extra-hepatic nucleic acid target is expressed in adipocytes.
Embodiment 11: The oligomeric compound of any of embodiments 1-10, wherein the extra-hepatic nucleic acid target is expressed in macrophages.
Embodiment 12: The oligomeric compound of any of embodiments 1-11, wherein the extra-hepatic nucleic acid target is expressed in cancer cells.
Embodiment 13: The oligomeric compound of any of embodiments 1-12, wherein the extra-hepatic nucleic acid target is expressed in tumor cells.
Embodiment 14: The oligomeric compound of any of embodiments 1-13, wherein the extra-hepatic nucleic acid target is expressed in smooth muscle cells
Embodiment 15: The oligomeric compound of any of embodiments 1-14, wherein the extra-hepatic nucleic acid target is expressed in heart muscle cells.
Embodiment 16: The oligomeric compound of any of embodiments 1-15, wherein the extra-hepatic nucleic acid target is expressed in lymphocytes.
Embodiment 17: The oligomeric compound of any of embodiments 1-16, wherein the extra-hepatic nucleic acid target is expressed in at least one extra-hepatic tissue selected from among: skeletal muscle, cardiac muscle, smooth muscle, adipose, white adipose, spleen, bone, intestine, adrenal, testes, ovary, pancreas, pituitary, prostate, skin, uterus, bladder, brain, glomerulus, distal tubular epithelium, breast, lung, heart, kidney, ganglion, frontal cortex, spinal cord, trigeminal ganglia, sciatic nerve, dorsal root ganglion, epididymal fat, diaphragm, and colon.
Embodiment 18: The oligomeric compound of any of embodiments 1-17, wherein the extra-hepatic nucleic acid target is expressed in at least two extra-hepatic tissues.
Embodiment 19: The oligomeric compound of any of embodiments 1-18, wherein the extra-hepatic nucleic acid target is expressed in at least three extra-hepatic tissues.
Embodiment 20: The oligomeric compound of any of embodiments 1-19, wherein the extra-hepatic nucleic acid target is expressed in at least four extra-hepatic tissues.
Embodiment 21: The oligomeric compound of any of embodiments 1-20, wherein the extra-hepatic nucleic acid target is expressed in skeletal muscle.
Embodiment 22: The oligomeric compound of any of embodiments 1-21, wherein the extra-hepatic nucleic acid target is expressed in cardiac muscle.
Embodiment 23: The oligomeric compound of any of embodiments 1-22, wherein the extra-hepatic nucleic acid target is expressed in smooth muscle.
Embodiment 24: The oligomeric compound of any of embodiments 1-23, wherein the extra-hepatic nucleic acid target is expressed in epididymal fat.
Embodiment 25: The oligomeric compound of any of embodiments 1-24, wherein the extra-hepatic nucleic acid target is expressed in white adipose tissue.
Embodiment 26: The oligomeric compound of any of embodiments 1-25, wherein the extra-hepatic nucleic acid target is expressed in the spleen.
Embodiment 27: The oligomeric compound of any of embodiments 1-26, wherein the extra-hepatic nucleic acid target is expressed in bone.
Embodiment 28: The oligomeric compound of any of embodiments 1-27, wherein the extra-hepatic nucleic acid target is expressed in bone marrow.
Embodiment 29: The oligomeric compound of any of embodiments 1-28, wherein the extra-hepatic nucleic acid target is expressed in the intestine.
Embodiment 30: The oligomeric compound of any of embodiments 1-29, wherein the extra-hepatic nucleic acid target is expressed in adrenal tissue.
Embodiment 31: The oligomeric compound of any of embodiments 1-30, wherein the extra-hepatic nucleic acid target is expressed in the testes.
Embodiment 32: The oligomeric compound of any of embodiments 1-31, wherein the extra-hepatic nucleic acid target is expressed in the ovaries.
Embodiment 33: The oligomeric compound of any of embodiments 1-32, wherein the extra-hepatic nucleic acid target is expressed in the pancreas.
Embodiment 34: The oligomeric compound of any of embodiments 1-33, wherein the extra-hepatic nucleic acid target is expressed in the pituitary.
Embodiment 35: The oligomeric compound of any of embodiments 1-34, wherein the extra-hepatic nucleic acid target is expressed in the prostate.
Embodiment 36: The oligomeric compound of any of embodiments 1-35, wherein the extra-hepatic nucleic acid target is expressed in the skin.
Embodiment 37: The oligomeric compound of any of embodiments 1-36, wherein the extra-hepatic nucleic acid target is expressed in the uterus.
Embodiment 38: The oligomeric compound of any of embodiments 1-37, wherein the extra-hepatic nucleic acid target is expressed in the bladder.
Embodiment 39: The oligomeric compound of any of embodiments 1-38, wherein the extra-hepatic nucleic acid target is expressed in the brain.
Embodiment 40: The oligomeric compound of any of embodiments 1-39, wherein the extra-hepatic nucleic acid target is expressed in the glomerulus.
Embodiment 41: The oligomeric compound of any of embodiments 1-40, wherein the extra-hepatic nucleic acid target is expressed in the distal tubular epithelium.
Embodiment 42: The oligomeric compound of any of embodiments 1-41, wherein the extra-hepatic nucleic acid target is expressed in the breast.
Embodiment 43: The oligomeric compound of any of embodiments 1-42, wherein the extra-hepatic nucleic acid target is expressed in the lung.
Embodiment 44: The oligomeric compound of any of embodiments 1-43, wherein the extra-hepatic nucleic acid target is expressed in the heart.
Embodiment 45: The oligomeric compound of any of embodiments 1-44, wherein the extra-hepatic nucleic acid target is expressed in the kidney.
Embodiment 46: The oligomeric compound of any of embodiments 1-45, wherein the extra-hepatic nucleic acid target is expressed in the colon.
Embodiment 47: The oligomeric compound of any of embodiments 1-46, wherein the extra-hepatic nucleic acid target is expressed in the ganglion.
Embodiment 48: The oligomeric compound of any of embodiments 1-47, wherein the extra-hepatic nucleic acid target is expressed in the frontal cortex.
Embodiment 49: The oligomeric compound of any of embodiments 1-48, wherein the extra-hepatic nucleic acid target is expressed in the spinal cord.
Embodiment 50: The oligomeric compound of any of embodiments 1-49, wherein the extra-hepatic nucleic acid target is expressed in the trigeminal ganglia.
Embodiment 51: The oligomeric compound of any of embodiments 1-50, wherein the extra-hepatic nucleic acid target is expressed in the sciatic nerve.
Embodiment 52: The oligomeric compound of any of embodiments 1-51, wherein the extra-hepatic nucleic acid target is expressed in the dorsal root ganglion.
Embodiment 53: The oligomeric compound of any of embodiments 1-52, wherein the extra-hepatic nucleic acid target is an endogenous RNA transcript.
Embodiment 54: The oligomeric compound of embodiment 53, wherein the RNA transcript is a pre-mRNA.
Embodiment 55: The oligomeric compound of embodiment 53, wherein the RNA transcript is an mRNA.
Embodiment 56: The oligomeric compound of embodiment 53, wherein the RNA transcript is a toxic RNA.
Embodiment 57: The oligomeric compound of embodiment 53, wherein the RNA transcript is a non-coding RNA.
Embodiment 58: The oligomeric compound of embodiment 56, wherein the RNA transcript is a microRNA.
Embodiment 59: The oligomeric compound of any of embodiments 1-52, wherein the extra-hepatic nucleic acid target is viral nucleic acid.
Embodiment 60: The oligomeric compound of any of embodiments 1-56, wherein the extra-hepatic nucleic acid target is selected from among: ATGL, CD40, TNF-α, CD36, DMPK, DNM2, DMD, DUX4, LMNA, ZFN9, SGLT2, and GCCR.
Embodiment 61: The oligomeric compound of any of embodiments 1-56, wherein the extra-hepatic nucleic acid target is selected from among: Androgen Receptor (AR), ANGPTL3, DGAT2, eIF4E, Factor XI, FGFR4, GCCR, GCGR, GHR, PTP1B, SMRT, STAT3, Them1, TRPV4, FTO, MC4R, TMEM18, KCTD15, GNPDA2, SH2B1, MTCH2, NEGR1, BDNF, ETVS, Leptin, leptin receptor, FAIM2, KCNMA1, MAF, NRXN3, TFAP2B, MSRA, AGPAT2, BSCL2, AKT2, PPARγ, LMNA, ZMPSTE24, DGAT1, TNFα, IL-6, Resistin, PAI-1, TBC1D1, METAP2, VEGF, AIF-1, JNK1, CB1, RIP140, TIF2, ANGPT1, ANGPT2, EIF4EBP2, CDK5, SLC13A5, Perilipin 1, Perilipin 2, Perilipin 3, Perilipin 4, HGF, GDF3, TNKs, KATNA1, ChREBP, ATF4, BASP-1, NNMT.
Embodiment 62: The oligomeric compound of any of embodiments 1-58, wherein the extra-hepatic nucleic acid target is other than any of: Androgen Receptor (AR), ANGPTL3, DGAT2, eIF4E, Factor XI, FGFR4, GCCR, GCGR, GHR, PTP1B, SMRT, STAT3, Them1, TRPV4, FTO, MC4R, TMEM18, KCTD15, GNPDA2, SH2B1, MTCH2, NEGR1, BDNF, ETVS, Leptin, leptin receptor, FAIM2, KCNMA1, MAF, NRXN3, TFAP2B, MSRA, AGPAT2, BSCL2, AKT2, PPARγ, LMNA, ZMPSTE24, DGAT1, TNFα, IL-6, Resistin, PAI-1, TBC1D1, METAP2, VEGF, AIF-1, JNK1, CB1, RIP140, TIF2, ANGPT1, ANGPT2, EIF4EBP2, CDK5, SLC13A5, Perilipin 1, Perilipin 2, Perilipin 3, Perilipin 4, HGF, GDF3, TNKs, KATNA1, ChREBP, ATF4, BASP-1, NNMT.
Embodiment 63: The oligomeric compound of any of embodiments 1-62, wherein the modified oligonucleotide has a nucleobase sequence that is at least 80% complementary to the nucleobase sequence of the extra-hepatic nucleic acid target, when measured across the entire nucleobase sequence of the modified oligonucleotide.
Embodiment 64: The oligomeric compound of embodiment 63, wherein the modified oligonucleotide has a nucleobase sequence that is at least 90% complementary to the nucleobase sequence of the extra-hepatic nucleic acid target, when measured across the entire nucleobase sequence of the modified oligonucleotide.
Embodiment 65: The oligomeric compound of embodiment 63, wherein the modified oligonucleotide has a nucleobase sequence that is 100% complementary to the nucleobase sequence of the extra-hepatic nucleic acid target, when measured across the entire nucleobase sequence of the modified oligonucleotide.
Embodiment 66: The oligomeric compound of any of embodiments 1-53, wherein the modified oligonucleotide has at least 8 contiguous nucleobases of any of the nucleobase sequences of SEQ ID NOs: 1, 2, 3, 4, 5, 6, 7, 8, 9, 10, 11, 12, 13, 14, 15, 16, 17, 18, 19, 20, 21, or 22.
Embodiment 67: The oligomeric compound of any of embodiments 1-53, wherein the modified oligonucleotide has at least 9 contiguous nucleobases of any of the nucleobase sequences of SEQ ID NOs: 1, 2, 3, 4, 5, 6, 7, 8, 9, 10, 11, 12, 13, 14, 15, 16, 17, 18, 19, 20, 21, or 22.
Embodiment 68: The oligomeric compound of any of embodiments 1-53, wherein the modified oligonucleotide has at least 10 contiguous nucleobases of any of the nucleobase sequences of SEQ ID NOs: 1, 2, 3, 4, 5, 6, 7, 8, 9, 10, 11, 12, 13, 14, 15, 16, 17, 18, 19, 20, 21, or 22.
Embodiment 69: The oligomeric compound of any of embodiments 1-53, wherein the modified oligonucleotide consists of the nucleobase sequence of any of SEQ ID NOs: 1, 2, 3, 4, 5, 6, 7, 8, 9, 10, 11, 12, 13, 14, 15, 16, 17, 18, 19, 20, 21, or 22.
Embodiment 70: The oligomeric compound of any of embodiments 1-53, wherein the modified oligonucleotide has at least 12 contiguous nucleobases of any of the nucleobase sequences of SEQ ID NOs: 1, 2, 3, 4, 5, 6, 7, 8, 9, 10, 11, 12, 13, 14, 15, 16, 17, 18, 19, 20, 21, or 22.
Embodiment 71: The oligomeric compound of any of embodiments 1-70, wherein the modified oligonucleotide does not have any DNA nucleosides.
Embodiment 72: The oligomeric compound of any of embodiments 1-71, wherein the modified oligonucleotide comprises at least one modified nucleoside.
Embodiment 73: The oligomeric compound of embodiment 72, wherein the modified oligonucleotide comprises a least one modified nucleoside comprising a modified sugar moiety.
Embodiment 74: The oligomeric compound of embodiment 73, wherein the modified oligonucleotide comprises at least one modified nucleoside comprising a bicyclic sugar moiety.
Embodiment 75: The oligomeric compound of embodiment 74, wherein the modified oligonucleotide comprises at least one modified nucleoside comprising a bicyclic sugar moiety having a 2′-4′ bridge, wherein the 2′-4′ bridge is selected from —O—CH₂—; and —O—CH(CH₃)—.
Embodiment 76: The oligomeric compound of any of embodiments 71-75, wherein the modified oligonucleotide comprises at least one modified nucleoside comprising a modified non-bicyclic sugar moiety.
Embodiment 77: The oligomeric compound of embodiment 76, wherein the modified oligonucleotide comprises at least one modified nucleoside comprising a non-bicyclic sugar moiety comprising a 2′-MOE or 2′-OMe.
Embodiment 78: The oligomeric compound of any of embodiments 71-77, wherein the modified oligonucleotide comprises at least one modified nucleoside comprising a sugar surrogate.
Embodiment 79: The oligomeric compound of embodiment 78, wherein the modified oligonucleotide comprises at least one modified nucleoside comprising a sugar surrogate selected from a morpholino, a PNA, a F-HNA, a THP, or a modified THP.
Embodiment 80: The oligomeric compound of any of embodiments 1-70 or 72-79, wherein the modified oligonucleotide has a sugar motif comprising:
   a 5′-region consisting of 1-5 linked 5′-nucleosides;
   a central region consisting of 6-10 linked central region nucleosides; and
   a 3′-region consisting of 1-5 linked 3′-region nucleosides; wherein each of the 5′-region nucleosides and each of the 3′-region comprises a modified sugar moiety and each of the central region nucleosides comprises an unmodified DNA sugar moiety.
Embodiment 81: The oligomeric compound of any of embodiments 1-80, wherein the modified oligonucleotide comprises at least one modified internucleoside linkage.
Embodiment 82: The oligomeric compound of embodiment 81, wherein each internucleoside linkage of the modified oligonucleotide is a modified internucleoside linkage.
Embodiment 83: The oligomeric compound of embodiment 81 or 82 wherein at least one internucleoside linkage is a phosphorothioate internucleoside linkage.
Embodiment 84: The oligomeric compound of embodiment 81 or 83 wherein the modified oligonucleotide comprises at least one unmodified phosphodiester internucleoside linkage.
Embodiment 85: The oligomeric compound of embodiment 84, wherein each internucleoside linkage is either an unmodified phosphodiester internucleoside linkage or a phosphorothioate internucleoside linkage.
Embodiment 86: The oligomeric compound of embodiment 82, wherein each internucleoside linkage is a phosphorothioate internucleoside linkage.
Embodiment 87: The oligomeric compound of any of embodiments 1-86, wherein the modified oligonucleotide comprises at least one modified nucleobase.
Embodiment 88: The oligomeric compound of embodiment 87, wherein the modified nucleobase is a 5-Me cytosine.
Embodiment 89: The oligomeric compound of any of embodiments 1-87 wherein each nucleobase of each nucleoside of the modified oligonucleotide is either an unmodified nucleobase or is 5-Me cytosine.
Embodiment 90: The oligomeric compound of any of embodiments 1-89, wherein the modified oligonucleotide consists of 12-22 linked nucleosides.
Embodiment 91: The oligomeric compound of any of embodiments 1-89, wherein the modified oligonucleotide consists of 12-20 linked nucleosides.
Embodiment 92: The oligomeric compound of any of embodiments 1-89, wherein the modified oligonucleotide consists of 14-20 linked nucleosides.
Embodiment 93: The oligomeric compound of any of embodiments 1-89, wherein the modified oligonucleotide consists of 16-20 linked nucleosides.
Embodiment 94: The oligomeric compound of any of embodiments 1-89, wherein the modified oligonucleotide consists of 18-20 linked nucleosides.
Embodiment 95: The oligomeric compound of any of embodiments 1-89, wherein the modified oligonucleotide consists of 20 linked nucleosides.
Embodiment 96: The oligomeric compound of any of embodiments 1-89, wherein the modified oligonucleotide consists of 19 linked nucleosides.
Embodiment 97: The oligomeric compound of any of embodiments 1-89, wherein the modified oligonucleotide consists of 18 linked nucleosides.
Embodiment 98: The oligomeric compound of any of embodiments 1-89, wherein the modified oligonucleotide consists of 17 linked nucleosides.
Embodiment 99: The oligomeric compound of any of embodiments 1-89, wherein the modified oligonucleotide consists of 16 linked nucleosides.
Embodiment 100: The oligomeric compound of any of embodiments 1-99, wherein the modified oligonucleotide is a single-stranded modified oligonucleotide.
Embodiment 101: The oligomeric compound of any of embodiments 1-99, wherein the oligomeric compound is paired with a second oligomeric compound to form a duplex.
Embodiment 102: The oligomeric compound of any of embodiments 1-101, wherein the conjugate linker comprises 1-5 linker-nucleosides.
Embodiment 103: The oligomeric compound of embodiment 102, wherein the conjugate linker comprises 3 linker-nucleosides.
Embodiment 104: The oligomeric compound of embodiment 103, wherein the 3 linker-nucleosides have a TCA motif.
Embodiment 105: The oligomeric compound of embodiment 96, wherein 1-5 linker-nucleosides do not comprise a TCA motif.
Embodiment 106: The oligomeric compound of any of embodiments 1-101, wherein the conjugate group does not comprise linker-nucleosides.
Embodiment 107: The oligomeric compound of any of embodiments 1-106, wherein the conjugate linker comprises a hexylamino group.
Embodiment 108: The oligomeric compound of any of embodiments 1-107, wherein the conjugate linker comprises a polyethylene glycol group.
Embodiment 109: The oligomeric compound of any of embodiments 1-108, wherein the conjugate linker comprises a triethylene glycol group.
Embodiment 110: The oligomeric compound of any of embodiments 1-109, wherein the conjugate linker comprises a phosphate group.
Embodiment 111: The oligomeric compound of any of embodiments 1-110, wherein the conjugate linker comprises:

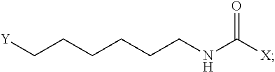

X directly or indirectly attaches to the conjugate moiety; and
Y directly or indirectly attaches to the modified oligonucleotide.
Embodiment 112: The oligomeric compound of embodiment 110, wherein X comprises 0.
Embodiment 113: The oligomeric compound of embodiment 111 or 112, wherein Y comprises a phosphate group.
Embodiment 114: The oligomeric compound of any of embodiments 1-110, wherein the conjugate linker comprises:

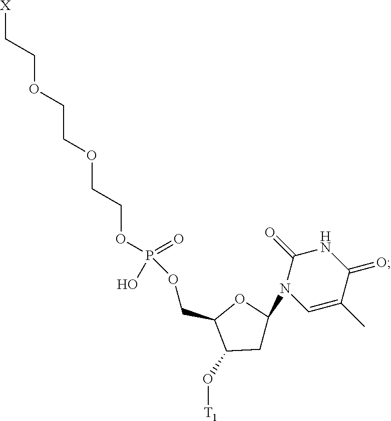

wherein m is 0 or 1;
X directly or indirectly attaches to the conjugate moiety; and
T₁ comprises a linking group, nucleoside, or a modified oligonucleotide.
Embodiment 115: The oligomeric compound of any of embodiments 1-110, wherein the conjugate linker comprises:

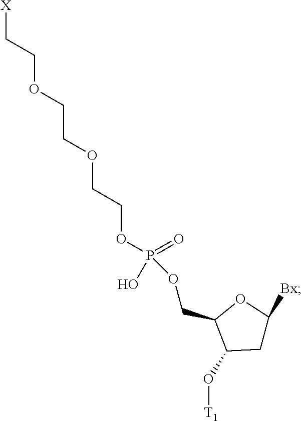

wherein m is 0 or 1;
X directly or indirectly attaches to the conjugate moiety; and
wherein T₁ comprises a nucleotide or a modified oligonucleotide; and Bₓ is a modified or unmodified nucleobase.
Embodiment 116: The oligomeric compound of any of embodiments 1-115, wherein the conjugate moiety comprises a lipophilic group.
Embodiment 117: The oligomeric compound of embodiment 116, wherein the lipophilic group is selected from among: cholesterol, C₁₀-C₂₆ saturated fatty acid, C₁₀-C₂₆ unsaturated fatty acid, C₁₀-C₂₆ alkyl, triglyceride, tocopherol, or cholic acid.
Embodiment 118: The oligomeric compound of embodiment 117, wherein the conjugate moiety is a saturated fatty acid or an unsaturated fatty acid.
Embodiment 119: The oligomeric compound of embodiment 117, wherein the conjugate moiety is C16 lipid.
Embodiment 120: The oligomeric compound of embodiment 117, wherein the conjugate moiety is C18 lipid.
Embodiment 121: The oligomeric compound of embodiment 117, wherein the conjugate moiety is C16 alkyl.
Embodiment 122: The oligomeric compound of embodiment 117, wherein the conjugate moiety is C18 alkyl.
Embodiment 123: The oligomeric compound of embodiment 117, wherein the conjugate moiety is cholesterol.
Embodiment 124: The oligomeric compound of embodiment 117, wherein the conjugate moiety is tocopherol.
Embodiment 125: The oligomeric compound of any of embodiments 1-124, wherein the conjugate group is attached to the modified oligonucleotide at the 5′-end of the modified oligonucleotide.
Embodiment 126: The oligomeric compound of any of embodiments 1-125, wherein the conjugate group is attached to the modified oligonucleotide at the 3′-end of the modified oligonucleotide.
Embodiment 127: The oligomeric compound of any of embodiments 1-126 comprising a terminal group.
Embodiment 128: An antisense compound consisting of the oligomeric compound of any of embodiments 1-127.
Embodiment 129: An antisense compound comprising the oligomeric compound of any of embodiments 1-127.
Embodiment 130: The antisense compound of embodiment 128 or 129 that is an RNase H antisense compound.
Embodiment 131: The antisense compound of embodiment 128 or 129 that is a single-stranded RNAi antisense compound.
Embodiment 132: The antisense compound of any of embodiments 128-131 that is capable of reducing the amount or activity of the extra-hepatic nucleic acid target by at least 20% when tested at a concentration of 1.0 nM in a standard cell assay.
Embodiment 133: The antisense compound of embodiment 132 that is capable of reducing the amount or activity of the extra-hepatic nucleic acid target by at least 40% in the standard cell assay.
Embodiment 134: The antisense compound of embodiment 132 that is capable of reducing the amount or activity of the extra-hepatic nucleic acid target by at least 80% in the standard cell assay.
Embodiment 135: The antisense compound of any of embodiments 128-134 that is capable of reducing the amount or activity of the extra-hepatic nucleic acid target in an extra-hepatic tissue by at least 20% when provided at a dose of 100 mg/kg in a standard animal experiment.
Embodiment 136: The antisense compound of embodiment 135 that is capable of reducing the amount or activity of the extra-hepatic nucleic acid target in the extra-hepatic tissue by at least 40%.
Embodiment 137: The antisense compound of embodiment 135 that is capable of reducing the amount or activity of the extra-hepatic nucleic acid target in the extra-hepatic tissue by at least 80%.
Embodiment 138: The antisense compound of embodiment 128 or 129 that alters the RNA processing of the extra-hepatic nucleic acid target.
Embodiment 139: A method comprising contacting a cell with the oligomeric compound of any of embodiments 1-126.
Embodiment 140: A method comprising contacting a cell with the antisense compound of any of embodiments 127-137.
Embodiment 141: A method of modulating the amount or activity of an extra-hepatic nucleic acid target in a cell comprising contacting the cell with the oligomeric compound or antisense compound of any of embodiments 1-137 and thereby modulating the amount or activity of the extra-hepatic nucleic acid target in the cell.
Embodiment 142: The method of embodiment 141, wherein the amount or activity of the extra-hepatic nucleic acid target is reduced.
Embodiment 143: The method of any of embodiments 139-142, wherein the cell is in vitro.
Embodiment 144: The method of any of embodiments 139-142, wherein the cell is in an animal.
Embodiment 145: The method of embodiment 144, wherein the animal is a human.
Embodiment 146: A pharmaceutical composition comprising an oligomeric compound of any embodiments 1-127 and a pharmaceutically acceptable carrier or diluent.
Embodiment 147: A pharmaceutical composition comprising an antisense compound of any of embodiments 128-138 and a pharmaceutically acceptable carrier or diluent.
Embodiment 148: A method comprising administering to an animal a pharmaceutical composition of embodiment 146 or 147.
Embodiment 149: A method of treating a disease associated with an extra-hepatic nucleic acid target comprising administering to an individual having or at risk for developing a disease associated with the extra-hepatic nucleic acid target a therapeutically effective amount of a pharmaceutical composition according to embodiment 146 or 147; and thereby treating the disease associated with the extra-hepatic nucleic acid target.
Embodiment 150: The method of embodiment 149, wherein the extra-hepatic nucleic acid target is selected from among: ATGL, CD40, CD36, DMPK, DNM2, DMD, DUX4, LMNA, ZFN9, SGLT2, or GCCR.
Embodiment 151: The method of embodiment 149, wherein the extra-hepatic nucleic acid target transcript is selected from among: Androgen Receptor (AR), ANGPTL3, DGAT2, eIF4E, Factor XI, FGFR4, GCCR, GCGR, GHR, PTP1B, SMRT, STAT3, Them1, TRPV4, FTO, MC4R, TMEM18, KCTD15, GNPDA2, SH2B1, MTCH2, NEGR1, BDNF, ETVS, Leptin, leptin receptor, FAIM2, KCNMA1, MAF, NRXN3, TFAP2B, MSRA, AGPAT2, BSCL2, AKT2, PPARγ, LMNA, ZMPSTE24, DGAT1, TNFα, IL-6, Resistin, PAI-1, TBC1D1, METAP2, VEGF, AIF-1, JNK1, CB1, RIP140, TIF2, ANGPT1, ANGPT2, EIF4EBP2, CDK5, SLC13A5, Perilipin 1, Perilipin 2, Perilipin 3, Perilipin 4, HGF, GDF3, TNKs, KATNA1, ChREBP, ATF4, BASP-1, NNMT.
Embodiment 152: The method of embodiment 149, wherein the extra-hepatic nucleic acid target transcript is not selected from among: Androgen Receptor (AR), ANGPTL3, DGAT2, eIF4E, Factor XI, FGFR4, GCCR, GCGR, GHR, PTP1B, SMRT, STAT3, Them1, TRPV4, FTO, MC4R, TMEM18, KCTD15, GNPDA2, SH2B1, MTCH2, NEGR1, BDNF, ETVS, Leptin, leptin receptor, FAIM2, KCNMA1, MAF, NRXN3, TFAP2B, MSRA, AGPAT2, BSCL2, AKT2, PPARγ, LMNA, ZMPSTE24, DGAT1, TNFα, IL-6, Resistin, PAI-1, TBC1D1, METAP2, VEGF, AIF-1, JNK1, CB1, RIP140, TIF2, ANGPT1, ANGPT2, EIF4EBP2, CDK5, SLC13A5, Perilipin 1, Perilipin 2, Perilipin 3, Perilipin 4, HGF, GDF3, TNKs, KATNA1, ChREBP, ATF4, BASP-1, NNMT.
Embodiment 153: The method of any of embodiments 149-152, wherein at least one symptom of a disease associated with an extra-hepatic nucleic acid target is ameliorated.
Embodiment 154: The method of any of embodiments 149-153, wherein the disease is selected from among: diabetes, metabolic syndrome, cardiac disease, muscular dystrophy, myotonic dystrophy, Becker muscular dystrophy, congenital muscular dystrophy, Duchenne muscular dystrophy, distal muscular dystrophy, Emery-Dreifuss muscular dystrophy, facioscapulohumeral muscular dystrophy, limb-girdle muscular dystrophy, or oculopharyngeal muscular dystrophy.
Embodiment 155: The method of any of embodiments 149-154 wherein the amount or activity of the extra-hepatic nucleic acid target is modulated in at least one tissue type other than liver.
Embodiment 156: The method of embodiment 149-155, wherein the amount of activity of the extra-hepatic nucleic acid target is modulated in at least two tissue types.
Embodiment 157: The method of embodiment 156, wherein at least one of the at least two tissue types is selected from among: liver, skeletal muscle, cardiac muscle, smooth muscle, adipose, white adipose, spleen, bone, intestine, adrenal, testes, ovary, pancreas, pituitary, prostate, skin, uterus, bladder, brain, glomerulus, distal tubular epithelium, breast, lung, heart, kidney, ganglion, frontal cortex, spinal cord, trigeminal ganglia, sciatic nerve, dorsal root ganglion, epididymal fat, diaphragm, and colon.
Embodiment 158: The method of embodiment 156, wherein at least two tissue types are selected from among: liver, skeletal muscle, cardiac muscle, smooth muscle, adipose, white adipose, spleen, bone, intestine, adrenal, testes, ovary, pancreas, pituitary, prostate, skin, uterus, bladder, brain, glomerulus, distal tubular epithelium, breast, lung, heart, kidney, ganglion, frontal cortex, spinal cord, trigeminal ganglia, sciatic nerve, dorsal root ganglion, epididymal fat, diaphragm, and colon.
Embodiment 159: A method of treating a multi-tissue disease or condition, comprising administering a therapeutically effective amount of the pharmaceutical composition of embodiment 146 or 147 to a subject, and thereby modulating the amount or activity of a target nucleic acid in two or more tissues.
Embodiment 160: A method of treating a disease or condition, comprising administering a therapeutically effective amount of the pharmaceutical composition of embodiment 146 or 147 to a subject, and thereby modulating the amount or activity of a target nucleic acid in two or more cell types.
Embodiment 161: A method of treating a multi-tissue disease or condition, comprising administering a therapeutically effective amount of the pharmaceutical composition of embodiment 146 or 147 to a subject, and thereby modulating the amount or activity of a target nucleic acid in two or more cell types.
Embodiment 162: The method of embodiment 160 or 161, wherein the two or more cell types are selected from among: hepatocytes, white fat cells, brown fat cells, adipocytes, macrophages, cancer cells, tumor cells, smooth muscle cells, lymphocytes, and heart muscle cells.
Embodiment 163: The method of embodiment 148, wherein the pharmaceutical composition is administered subcutaneously.
Embodiment 164: The method of embodiment 148, wherein the pharmaceutical composition is administered intravenously.
Embodiment 165: The method of embodiment 148, wherein the pharmaceutical composition is administered by parenteral administration.
Embodiment 166: The method of embodiment 148, wherein the pharmaceutical composition is administered by intraperitoneal administration.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well known and commonly used in the art. Where permitted, all patents, applications, published applications and other publications and other data referred to throughout in the disclosure are incorporated by reference herein in their entirety.

Unless otherwise indicated, the following terms have the following meanings: “2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

“2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2′-substituent group other than H or OH.

“Antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound. In certain embodiments, antisense activity is a change in splicing of a pre-mRNA nucleic acid target. In certain embodiments, antisense activity is an increase in the amount or expression of a target nucleic acid or protein encoded by such target nucleic acid compared to target nucleic acid levels or target protein levels in the absence of the antisense compound.

“Antisense compound” means a compound comprising an antisense oligonucleotide and optionally one or more additional features, such as a conjugate group or terminal group.

“Antisense oligonucleotide” means an oligonucleotide that (1) has a nucleobase sequence that is at least partially complementary to a target nucleic acid and that (2) is capable of producing an antisense activity in a cell or animal.

“Ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom.

“Bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

“Branching group” means a group of atoms having at least 3 positions that are capable of forming covalent linkages to at least 3 groups. In certain embodiments, a branching group provides a plurality of reactive sites for connecting tethered ligands to an oligonucleotide via a conjugate linker and/or a cleavable moiety.

“Cell-targeting moiety” means a conjugate group or portion of a conjugate group that is capable of binding to a particular cell type or particular cell types.

“Cleavable moiety” means a bond or group of atoms that is cleaved under physiological conditions, for example, inside a cell, an animal, or a human.

“Complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include, but unless otherwise specific are not limited to, adenine (A) and thymine (T), adenine (A) and uracil (U), cytosine (C) and guanine (G), 5-methyl cytosine (IT) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

“Conjugate group” means a group of atoms that is directly or indirectly attached to an oligonucleotide. Conjugate groups include a conjugate moiety and a conjugate linker that attaches the conjugate moiety to the oligonucleotide.

“Conjugate linker” means a group of atoms comprising at least one bond that connects a conjugate moiety to an oligonucleotide.

“Conjugate moiety” means a group of atoms that is attached to an oligonucleotide via a conjugate linker.

“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

“Duplex” means two oligomeric compounds that are paired. In certain embodiments, the two oligomeric compounds are paired via hybridization of complementary nucleobases.

“Extra-hepatic cell type” means a cell type that is not a hepatocyte.

“Extra-hepatic nucleic acid target” means a target nucleic acid that is expressed in tissues other than liver. In certain embodiments, extra-hepatic nucleic acid targets are not expressed in the liver or not expressed in the liver at a significant level. In certain embodiments, extra-hepatic nucleic acid targets are expressed outside the liver and also in the liver.

“Extra hepatic disease” means a disease or condition where one or more symptoms or causes of the disease or condition occur in tissues other than liver.

“Extra-hepatic tissue” means a tissue other than liver.

“Fully modified” in reference to a modified oligonucleotide means a modified oligonucleotide in which each sugar moiety is modified. “Uniformly modified” in reference to a modified oligonucleotide means a fully modified oligonucleotide in which each sugar moiety is the same. For example, the nucleosides of a uniformly modified oligonucleotide can each have a 2′-MOE modification but different nucleobase modifications, and the internucleoside linkages may be different.

“Gapmer” means an antisense oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Heart disease” means any disease or condition where one or more symptoms or causes of the disease or condition manifests in the heart. For example, in certain embodiments, a heart disease may be caused by a particular nucleic acid transcript expressed in a cardiomyocyte, endothelial cell, fibroblast, or macrophage located in the heart. In certain embodiments a heart disease may be caused or associated with a particular nucleic acid target or nucleic acid transcript expressed in the heart.

“Hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

“Inhibiting the expression or activity” refers to a reduction or blockade of the expression or activity relative to the expression of activity in an untreated or control sample and does not necessarily indicate a total elimination of expression or activity.

“Internucleoside linkage” means a group or bond that forms a covalent linkage between adjacent nucleosides in an oligonucleotide. As used herein “modified internucleoside linkage” means any internucleoside linkage other than a naturally occurring, phosphate internucleoside linkage. Non-phosphate linkages are referred to herein as modified internucleoside linkages. “Phosphorothioate linkage” means a modified phosphate linkage in which one of the non-bridging oxygen atoms is replaced with a sulfur atom. A phosphorothioate internucleoside linkage is a modified internucleoside linkage.

“Linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

“Lipophilic group” or “lipophilic” in reference to a chemical group means a group of atoms that is more soluble in lipids or organic solvents than in water and/or has a higher affinity for lipids than for water. In certain embodiments, lipophilic groups comprise a lipid. As used herein “lipid” means a molecule that is not soluble in water or is less soluble in water than in organic solvents. In certain embodiments, compounds of the present invention comprise lipids selected from saturated or unsaturated fatty acids, steroids, fat soluble vitamins, phospholipids, sphingolipids, hydrocarbons, mono-, di-, and tri-glycerides, and synthetic derivatives thereof.

“Non-bicyclic modified sugar” or “non-bicyclic modified sugar moiety” means a modified sugar moiety that comprises a modification, such as a substitutent, that does not form a bridge between two atoms of the sugar to form a second ring.

“Linked nucleosides” are nucleosides that are connected in a continuous sequence (i.e. no additional nucleosides are present between those that are linked).

“Mismatch” or “non-complementary” means a nucleobase of a first oligonucleotide that is not complementary with the corresponding nucleobase of a second oligonucleotide or target nucleic acid when the first and second oligomeric compound are aligned.

“MOE” means methoxyethyl. “2′-MOE” means a —OCH₂CH₂OCH₃ group at the 2′ position of a furanosyl ring.

“Motif” means the pattern of unmodified and/or modified sugar moieties, nucleobases, and/or internucleoside linkages, in an oligonucleotide.

“Multi-tissue disease or condition” means a disease or condition affects or is effected by more than one tissue. In treating a multi-tissue disease or condition, it is desirable to affect more than one tissue type. In certain embodiments, treatment of disease or condition may be enhanced by treating the disease or condition in multiple tissues. For example, in certain embodiments, a disease or condition may manifest itself in the liver tissue and the muscle tissue. In certain embodiments, treating the disease or condition in the liver tissue and the muscle tissue will be more effective than treating the disease in either the liver tissue or the muscle tissue.

“Naturally occurring” means found in nature.

“Nucleobase” means an unmodified nucleobase or a modified nucleobase. As used herein a “an “unmodified nucleobase” is adenine (A), thymine (T), cytosine (C), uracil (U), and guanine (G). As used herein, a “modified nucleobase” is a group of atoms other than unmodified A, T, C, U, or G capable of pairing with at least one unmodified nucleobase. A universal base is a modified nucleobase that can pair with any one of the five unmodified nucleobases. As used herein, “nucleobase sequence” means the order of contiguous nucleobases in a nucleic acid or oligonucleotide independent of any sugar or internucleoside linkage modification.

“Nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase.

“Oligomeric compound” means a compound consisting of an oligonucleotide and optionally one or more additional features, such as a conjugate group or terminal group.

“Oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

“Pharmaceutically acceptable carrier or diluent” means any substance suitable for use in administering to an animal Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water; sterile saline; or sterile buffer solution.

“Pharmaceutically acceptable salts” means physiologically and pharmaceutically acceptable salts of compounds, such as oligomeric compounds, i.e., salts that retain the desired biological activity of the parent compound and do not impart undesired toxicological effects thereto.

“Pharmaceutical composition” means a mixture of substances suitable for administering to a subject. For example, a pharmaceutical composition may comprise an antisense compound and a sterile aqueous solution. In certain embodiments, a pharmaceutical composition shows activity in free uptake assay in certain cell lines.

“Phosphorus moiety” means a group of atoms comprising a phosphorus atom. In certain embodiments, a phosphorus moiety comprises a mono-, di-, or tri-phosphate, or phosphorothioate.

“Prodrug” means a therapeutic agent in a form outside the body that is converted to a different form within the body or cells thereof. Typically conversion of a prodrug within the body is facilitated by the action of an enzymes (e.g., endogenous or viral enzyme) or chemicals present in cells or tissues and/or by physiologic conditions.

“RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense oligonucleotides that act through RNase H.

“Single-stranded” in reference to an oligomeric compound means such a compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex, in which case it would no longer be single-stranded.

“Standard cell assay” means the assay described in Example 1 and reasonable variations thereof

“Standard in vivo experiment” means the procedure described in Example 5 and reasonable variations thereof.

“Sugar moiety” means an unmodified sugar moiety or a modified sugar moiety. As used herein, “unmodified sugar moiety” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars. As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

“Target nucleic acid” means a naturally occurring, identified nucleic acid. In certain embodiments, target nucleic acids are endogenous cellular nucleic acids, including, but not limited to RNA transcripts, pre-mRNA, mRNA, microRNA. In certain embodiments, target nucleic acids are viral nucleic acids. In certain embodiments, target nucleic acids are nucleic acids that an antisense compound is designed to affect.

“Target region” means a portion of a target nucleic acid to which an antisense compound is designed to hybridize.

“TCA motif” means three nucleosides having the nucleobase sequence TCA (5′-3′). Such nucleosides may have modified sugar moieties and/or modified internucleosides linkages. Unless otherwise indicated, the nucleosides of TCA motifs comprise unmodified 2′-deoxy sugar moieties and unmodified phosphodiester internucleoside linkages.

“Terminal group” means a chemical group or group of atoms that is covalently linked to a terminus of an oligonucleotide.

“Skeletal muscle target” means a nucleic acid transcript for which there is some desired therapeutic benefit from modulating the amount or activity of the nucleic acid transcript in skeletal muscle tissue. For example, a given nucleic acid transcript may be expressed in multiple tissues, however one or more therapeutic benefit is achieved when the amount or activity of the target nucleic acid is modulated in skeletal muscle tissue.

“Cardiac muscle target” means a nucleic acid transcript for which there is some desired therapeutic benefit from modulating the amount or activity of the nucleic acid transcript in cardiac muscle tissue. For example, a given nucleic acid transcript may be expressed in multiple tissues, however one or more therapeutic benefit is achieved when the amount or activity of the target nucleic acid is modulated in cardiac muscle tissue.

“Smooth muscle target” means a nucleic acid transcript for which there is some desired therapeutic benefit from modulating the amount or activity of the nucleic acid transcript in smooth muscle tissue. For example, a given nucleic acid transcript may be expressed in multiple tissues, however one or more therapeutic benefit is achieved when the amount or activity of the target nucleic acid is modulated in smooth muscle tissue.

“Epididymal fat” means a nucleic acid transcript for which there is some desired therapeutic benefit from modulating the amount or activity of the nucleic acid transcript in epididymal fat tissue. For example, a given nucleic acid transcript may be expressed in multiple tissues, however one or more therapeutic benefit is achieved when the amount or activity of the target nucleic acid is modulated in epididymal fat tissue.

“White adipose tissue target” means a nucleic acid transcript for which there is some desired therapeutic benefit from modulating the amount or activity of the nucleic acid transcript in white adipose tissue. For example, a given nucleic acid transcript may be expressed in multiple tissues, however one or more therapeutic benefit is achieved when the amount or activity of the target nucleic acid is modulated in white adipose tissue.

“Spleen target” means a nucleic acid transcript for which there is some desired therapeutic benefit from modulating the amount or activity of the nucleic acid transcript in spleen tissue. For example, a given nucleic acid transcript may be expressed in multiple tissues, however one or more therapeutic benefit is achieved when the amount or activity of the target nucleic acid is modulated in spleen tissue.

“Bone” means a nucleic acid transcript for which there is some desired therapeutic benefit from modulating the amount or activity of the nucleic acid transcript in bone tissue. For example, a given nucleic acid transcript may be expressed in multiple tissues, however one or more therapeutic benefit is achieved when the amount or activity of the target nucleic acid is modulated in bone tissue.

“Bone marrow target” means a nucleic acid transcript for which there is some desired therapeutic benefit from modulating the amount or activity of the nucleic acid transcript in bone marrow tissue. For example, a given nucleic acid transcript may be expressed in multiple tissues, however one or more therapeutic benefit is achieved when the amount or activity of the target nucleic acid is modulated in bone marrow tissue.

“Intestine target” means a nucleic acid transcript for which there is some desired therapeutic benefit from modulating the amount or activity of the nucleic acid transcript in intestinal tissue. For example, a given nucleic acid transcript may be expressed in multiple tissues, however one or more therapeutic benefit is achieved when the amount or activity of the target nucleic acid is modulated in intestinal tissue.

“Adrenal tissue target” means a nucleic acid transcript for which there is some desired therapeutic benefit from modulating the amount or activity of the nucleic acid transcript in adrenal tissue. For example, a given nucleic acid transcript may be expressed in multiple tissues, however one or more therapeutic benefit is achieved when the amount or activity of the target nucleic acid is modulated in adrenal tissue.

“Testes target” means a nucleic acid transcript for which there is some desired therapeutic benefit from modulating the amount or activity of the nucleic acid transcript in testicular tissue. For example, a given nucleic acid transcript may be expressed in multiple tissues, however one or more therapeutic benefit is achieved when the amount or activity of the target nucleic acid is modulated in testicular tissue.

“Ovaries target” means a nucleic acid transcript for which there is some desired therapeutic benefit from modulating the amount or activity of the nucleic acid transcript in ovarian tissue. For example, a given nucleic acid transcript may be expressed in multiple tissues, however one or more therapeutic benefit is achieved when the amount or activity of the target nucleic acid is modulated in ovarian tissue.

“Pancreas target” means a nucleic acid transcript for which there is some desired therapeutic benefit from modulating the amount or activity of the nucleic acid transcript in pancreatic tissue. For example, a given nucleic acid transcript may be expressed in multiple tissues, however one or more therapeutic benefit is achieved when the amount or activity of the target nucleic acid is modulated in pancreatic tissue.

“Pituitary” means a nucleic acid transcript for which there is some desired therapeutic benefit from modulating the amount or activity of the nucleic acid transcript in pituitary tissue. For example, a given nucleic acid transcript may be expressed in multiple tissues, however one or more therapeutic benefit is achieved when the amount or activity of the target nucleic acid is modulated in pituitary tissue.

“Prostate target” means a nucleic acid transcript for which there is some desired therapeutic benefit from modulating the amount or activity of the nucleic acid transcript in prostate tissue. For example, a given nucleic acid transcript may be expressed in multiple tissues, however one or more therapeutic benefit is achieved when the amount or activity of the target nucleic acid is modulated in prostate tissue.

“Skin target” means a nucleic acid transcript for which there is some desired therapeutic benefit from modulating the amount or activity of the nucleic acid transcript in skin tissue. For example, a given nucleic acid transcript may be expressed in multiple tissues, however one or more therapeutic benefit is achieved when the amount or activity of the target nucleic acid is modulated in skin tissue.

“Uterus target” means a nucleic acid transcript for which there is some desired therapeutic benefit from modulating the amount or activity of the nucleic acid transcript in uterus tissue. For example, a given nucleic acid transcript may be expressed in multiple tissues, however one or more therapeutic benefit is achieved when the amount or activity of the target nucleic acid is modulated in uterus tissue.

“Bladder target” means a nucleic acid transcript for which there is some desired therapeutic benefit from modulating the amount or activity of the nucleic acid transcript in bladder tissue. For example, a given nucleic acid transcript may be expressed in multiple tissues, however one or more therapeutic benefit is achieved when the amount or activity of the target nucleic acid is modulated in bladder tissue.

“Brain target” means a nucleic acid transcript for which there is some desired therapeutic benefit from modulating the amount or activity of the nucleic acid transcript in brain tissue. For example, a given nucleic acid transcript may be expressed in multiple tissues, however one or more therapeutic benefit is achieved when the amount or activity of the target nucleic acid is modulated in brain tissue.

“Glomerulus target” means a nucleic acid transcript for which there is some desired therapeutic benefit from modulating the amount or activity of the nucleic acid transcript in glomerulus tissue. For example, a given nucleic acid transcript may be expressed in multiple tissues, however one or more therapeutic benefit is achieved when the amount or activity of the target nucleic acid is modulated in glomerulus tissue.

“Distal tubular epithelium target” means a nucleic acid transcript for which there is some desired therapeutic benefit from modulating the amount or activity of the nucleic acid transcript in distal tubular epithelium tissue. For example, a given nucleic acid transcript may be expressed in multiple tissues, however one or more therapeutic benefit is achieved when the amount or activity of the target nucleic acid is modulated in distal tubular epithelium tissue.

“Breast target” means a nucleic acid transcript for which there is some desired therapeutic benefit from modulating the amount or activity of the nucleic acid transcript in breast tissue. For example, a given nucleic acid transcript may be expressed in multiple tissues, however one or more therapeutic benefit is achieved when the amount or activity of the target nucleic acid is modulated in breast tissue.

“Lung target” means a nucleic acid transcript for which there is some desired therapeutic benefit from modulating the amount or activity of the nucleic acid transcript in lung tissue. For example, a given nucleic acid transcript may be expressed in multiple tissues, however one or more therapeutic benefit is achieved when the amount or activity of the target nucleic acid is modulated in lung tissue.

“Heart target” means a nucleic acid transcript for which there is some desired therapeutic benefit from modulating the amount or activity of the nucleic acid transcript in heart tissue. For example, a given nucleic acid transcript may be expressed in multiple tissues, however one or more therapeutic benefit is achieved when the amount or activity of the target nucleic acid is modulated in heart tissue.

“Kidney target” means a nucleic acid transcript for which there is some desired therapeutic benefit from modulating the amount or activity of the nucleic acid transcript in kidney tissue. For example, a given nucleic acid transcript may be expressed in multiple tissues, however one or more therapeutic benefit is achieved when the amount or activity of the target nucleic acid is modulated in kidney tissue.

“Colon target” means a nucleic acid transcript for which there is some desired therapeutic benefit from modulating the amount or activity of the nucleic acid transcript in colon tissue. For example, a given nucleic acid transcript may be expressed in multiple tissues, however one or more therapeutic benefit is achieved when the amount or activity of the target nucleic acid is modulated in colon tissue.

“Ganglion target” means a nucleic acid transcript for which there is some desired therapeutic benefit from modulating the amount or activity of the nucleic acid transcript in ganglion tissue. For example, a given nucleic acid transcript may be expressed in multiple tissues, however one or more therapeutic benefit is achieved when the amount or activity of the target nucleic acid is modulated in ganglion tissue.

“Frontal cortex target” means a nucleic acid transcript for which there is some desired therapeutic benefit from modulating the amount or activity of the nucleic acid transcript in frontal cortex tissue. For example, a given nucleic acid transcript may be expressed in multiple tissues, however one or more therapeutic benefit is achieved when the amount or activity of the target nucleic acid is modulated in frontal cortex tissue.

“Spinal cord target” means a nucleic acid transcript for which there is some desired therapeutic benefit from modulating the amount or activity of the nucleic acid transcript in spinal cord tissue. For example, a given nucleic acid transcript may be expressed in multiple tissues, however one or more therapeutic benefit is achieved when the amount or activity of the target nucleic acid is modulated in spinal cord tissue.

“Trigeminal ganglia target” means a nucleic acid transcript for which there is some desired therapeutic benefit from modulating the amount or activity of the nucleic acid transcript in trigeminal ganglia tissue. For example, a given nucleic acid transcript may be expressed in multiple tissues, however one or more therapeutic benefit is achieved when the amount or activity of the target nucleic acid is modulated in trigeminal ganglia tissue.

“Sciatic nerve target” means a nucleic acid transcript for which there is some desired therapeutic benefit from modulating the amount or activity of the nucleic acid transcript in sciatic nerve tissue. For example, a given nucleic acid transcript may be expressed in multiple tissues, however one or more therapeutic benefit is achieved when the amount or activity of the target nucleic acid is modulated in sciatic nerve tissue.

“Dorsal root ganglion target” means a nucleic acid transcript for which there is some desired therapeutic benefit from modulating the amount or activity of the nucleic acid transcript in dorsal root ganglion tissue. For example, a given nucleic acid transcript may be expressed in multiple tissues, however one or more therapeutic benefit is achieved when the amount or activity of the target nucleic acid is modulated in dorsal root ganglion tissue.

I. Certain Oligonucleotides

In certain embodiments, the invention provides oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA (i.e., comprise at least one modified nucleoside (comprising a modified sugar moiety and/or a modified nucleobase) and/or at least one modified internucleoside linkage).

A. Certain Modified Nucleosides

Modified nucleosides comprise a modified sugar moiety or a modified nucleobase or both a modifed sugar moiety and a modified nucleobase.

1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more acyclic substituent, including but not limited to substituents at the 2′, 4′, and/or 5′ positions. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂S CH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836).

In certain embodiments, a 2′-substituted nucleoside or 2′-non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or 2′-non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or 2′-non-bicyclic modified nucleoside comprises a sugar moiety comprising a non-bridging 2′-substituent group selected from: F, OCH₃, and OCH₂CH₂OCH₃.

Nucleosides comprising modified sugar moieties, such as non-bicyclic modified sugar moieties, may be referred to by the position(s) of the substitution(s) on the sugar moiety of the nucleoside. For example, nucleosides comprising 2′-substituted or 2-modified sugar moieties are referred to as 2′-substituted nucleosides or 2-modified nucleosides.

Certain modifed sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J Org. Chem., 1998, 63, 10035-10039; Srivastava et al., J Am. Chem. Soc., 20017, 129, 8362-8379; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

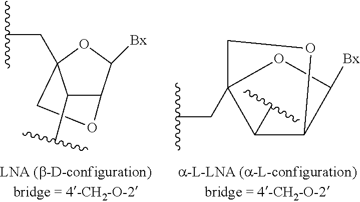

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. &Med. Chem. 2002, 10, 841-854), fluoro HNA:

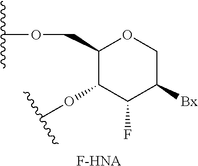

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

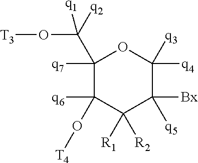

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

each of R₁ and R₂ is independently selected from among: hydrogen, halogen, substituted or unsubstituted alkoxy, NJ₁J₂, SJ₃, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

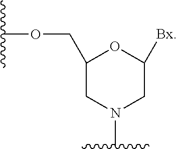

In certain embodiments, morpholinos may be modified, for example by adding or altering various substituent groups from the above morpholino structure. Such sugar surrogates are referred to herein as “modifed morpholinos.”

In certain embodiments, sugar surrogates comprise acyclic moieites. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides).

1. Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manohara et al., US2003/0158403; Manoharan et al., US2003/0175906; Dinh et al., U.S. Pat. No. 4,845,205; Spielvogel et al., U.S. Pat. No. 5,130,302; Rogers et al., U.S. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

B. Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

C. Certain Motifs

In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, the wings of a gapmer comprise 2-5 nucleosides. In certain embodiments, the wings of a gapmer comprise 3-5 nucleosides. In certain embodiments, the nucleosides of a gapmer are all modified nucleosides.

In certain embodiments, the gap of a gapmer comprises 7-12 nucleosides. In certain embodiments, the gap of a gapmer comprises 7-10 nucleosides. In certain embodiments, the gap of a gapmer comprises 8-10 nucleosides. In certain embodiments, the gap of a gapmer comprises 10 nucleosides. In certain embodiment, each nucleoside of the gap of a gapmer is an unmodified 2′-deoxy nucleoside.

In certain embodiments, the gapmer is a deoxy gapmer. In such embodiments, the nucleosides on the gap side of each wing/gap junction are unmodified 2′-deoxy nucleosides and the nucleosides on the wing sides of each wing/gap junction are modified nucleosides. In certain such embodiments, each nucleoside of the gap is an unmodified 2′-deoxy nucleoside. In certain such embodiments, each nucleoside of each wing is a modified nucleoside.

In certain embodiments, modified oligonucleotides comprise or consist of a region having a fully modified sugar motif. In such embodiments, each nucleoside of the fully modified region of the modified oligonucleotide comprises a modified sugar moiety. In certain such embodiments, each nucleoside to the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the sugar motif of a modified oligonucleotide is a gapmer and the internucleoside linkages within the gap are all modified. In certain such embodiments, some or all of the internucleoside linkages in the wings are unmodified phosphate linkages. In certain embodiments, the terminal internucleoside linkages are modified.

D. Certain Lengths

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides

E. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Furthermore, in certain instances, an oligonucleotide is described by an overall length or range and by lengths or length ranges of two or more regions (e.g., a regions of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists if of 15-20 linked nucleosides and has a sugar motif consisting of three regions, A, B, and C, wherein region A consists of 2-6 linked nucleosides having a specified sugar motif, region B consists of 6-10 linked nucleosides having a specified sugar motif, and region C consists of 2-6 linked nucleosides having a specified sugar motif. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of the overall length of the modified oligonucleotide (20). Herein, if a description of an oligonucleotide is silent with respect to one or more parameter, such parameter is not limited. Thus, a modified oligonucleotide described only as having a gapmer sugar motif without further description may have any length, internucleoside linkage motif, and nucleobase motif. Unless otherwise indicated, all modifications are independent of nucleobase sequence.

F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 90%, at least 95%, or 100% complementary to the second oligonucleotide or nucleic acid, such as a target nucleic acid.

II. Certain Oligomeric Compounds

In certain embodiments, the invention provides oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

Most oligomeric compounds are metabolized in the liver or kidneys, which can reduce the half life of the oligomeric compound in a subject. For example, in certain embodiments, an oligomeric compound administered to a subject may distribute to the kidneys and then be excreted out in the subject's urine. In another embodiments, Conjugating an oligomeric compound an oligomeric compound administered to a subject may be metabolized in the liver. In certain embodiments, an oligomeric compound administered to a subject is both metabolized by the liver and excreted out through the kidneys. In certain embodiments, the present disclosure provides oligomeric compounds comprising a modified oligonucleotide and a conjugate group, wherein the conjugate group enhances delivery of the modified oligonucleotide. In certain embodiments, the conjugate group enhances delivery of the modified oligonucleotide to a tissue selected from among: skeletal muscle, cardiac muscle, smooth muscle, adipose, white adipose, spleen, bone, intestine, adrenal, testes, ovary, pancreas, pituitary, prostate, skin, uterus, bladder, brain, glomerulus, distal tubular epithelium, breast, lung, heart, kidney, ganglion, frontal cortex, spinal cord, trigeminal ganglia, sciatic nerve, dorsal root ganglion, epididymal fat, diaphragm, and colon.

Oligomeric compounds typically show good uptake in hepatocytes. In certain embodiments, the present disclosure provides oligomeric compounds comprising a modified oligonucleotide and a conjugate group, wherein the conjugate group enhances uptake in a particular cell type. In certain embodiments, the conjugate group enhances uptake in macrophages. In certain embodiments, the conjugate group enhances uptake in cardiomyocytes. In certain embodiments, the conjugate group enhances uptake in fibroblasts. In certain embodiments, the conjugate group enhances uptake in endothelial cells. In certain embodiments, the conjugate group enhances uptake in heart cells.

1. Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises a compound found endogenously in a subject. For example, in certain embodiments, the conjugate may be a steroid, such as cholesterol. Although cholesterol is endogenously produced in a subject and has certain physiological activities, cholesterol may be used as a conjugate to alter or improve one or more properties of a modified oligonucleotide. For example, cholesterol conjugated to a modified oligonucleotide may increase the modified oligonucleotide's binding affinity for a given protein, such as HDL

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

In certain embodiments, conjugate moieties impart a new property on the attached oligonucleotide, which may alter the oligonucleotides distribution or pharmacokinetic profile. For example, certain conjugate moieties selected from among lipids, vitamins, steroids, C₅-C₃₀ saturated alkyl groups, C₅-C₃₀ unsaturated alkyl groups, fatty acids, or lipophilic groups may increase the distribution of an oligonucleotide to various tissues or organs within a subject. In certain embodiments, certain conjugate moieties selected from among lipids, vitamins, steroids, C₅-C₃₀ saturated alkyl groups, C₅-C₃₀ unsaturated alkyl groups, fatty acids, or lipophilic groups increase affinity for an oligonucleotide with one or more serum proteins, such as albumin. In certain embodiments, certain conjugate moieties selected from among lipids, vitamins, steroids, C₅-C₃₀ saturated alkyl groups, C₅-C₃₀ unsaturated alkyl groups, fatty acids, or lipophilic groups increase affinity for an oligonucleotide to an extra-hepatic tissue. In certain embodiments, this allows for conjugated oligonucleotides to have longer half lives because the less of the conjugated oligonucleotide is metabolized in the liver.

In certain embodiments, certain conjugate moieties are selected from among lipids, vitamins, steroids, C₅-C₃₀ saturated alkyl groups, C₅-C₃₀ unsaturated alkyl groups, fatty acids, or lipophilic groups increase affinity for an extra-hepatic tissue selected from among: skeletal muscle, cardiac muscle, smooth muscle, adipose, white adipose, spleen, bone, intestine, adrenal, testes, ovary, pancreas, pituitary, prostate, skin, uterus, bladder, brain, glomerulus, distal tubular epithelium, breast, lung, heart, kidney, ganglion, frontal cortex, spinal cord, trigeminal ganglia, sciatic nerve, dorsal root ganglion, epididymal fat, diaphragm, pancreas, and colon.

In certain embodiments, a conjugate moiety is selected from among:

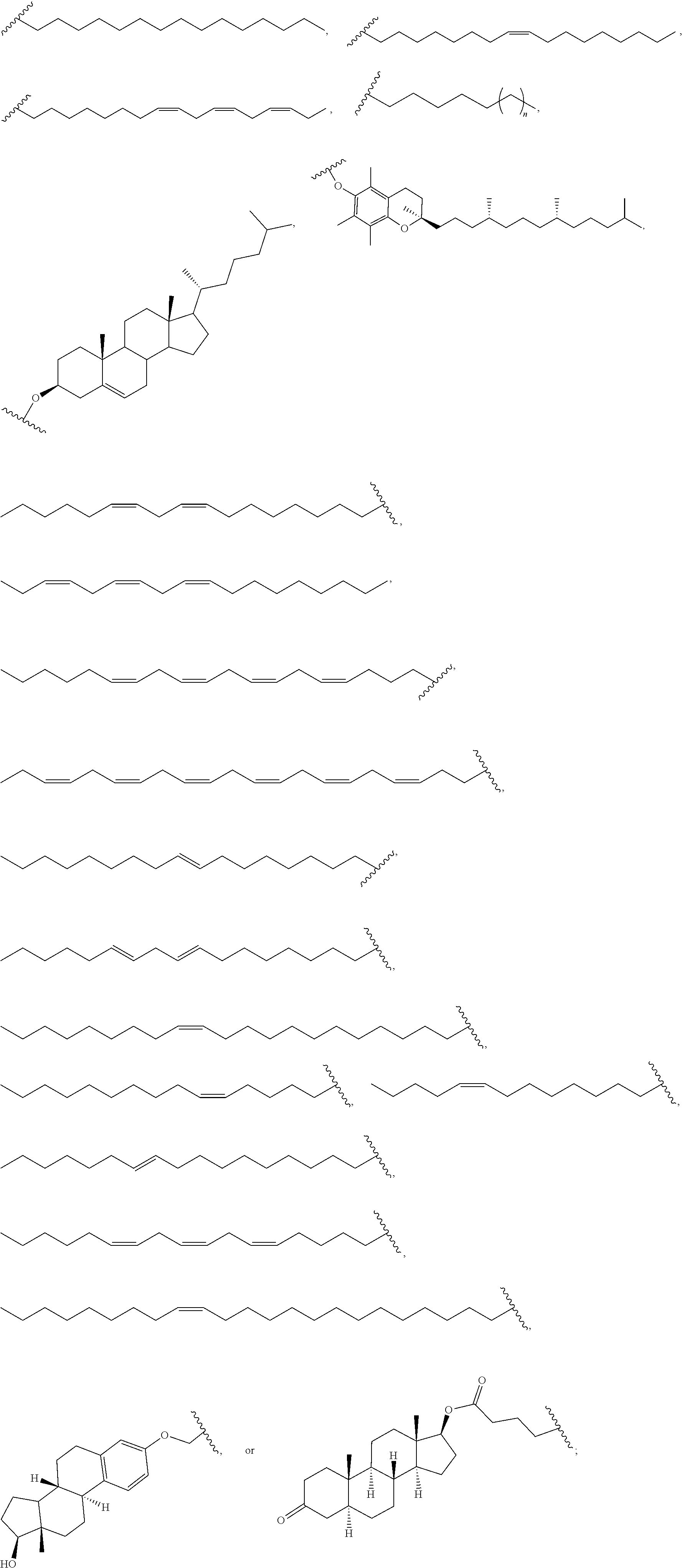

wherein n is selected from among: 1, 2, 3, 4, 5, 6, 7, 8, 9, 10, 11, 12, 13, 14, 15, or 16.

2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif.

In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

III. Certain Antisense Compounds

In certain embodiments, the present invention provides antisense compounds, which comprise or consist of an oligomeric compound comprising an antisense oliognucleotide, having a nucleobase sequences complementary to that of a target nucleic acid. In certain embodiments, antisense compounds are single-stranded. Such single-stranded antisense compounds typically comprise or consist of an oligomeric compound that comprises or consists of a modified oligonucleotide and optionally a conjugate group. In certain embodiments, antisense compounds are double-stranded. Such double-stranded antisense compounds comprise a first oligomeric compound having a region complementary to a target nucleic acid and a second oligomeric compound having a region complementary to the first oligomeric compound. The first oligomeric compound of such double stranded antisense compounds typically comprises or consists of a modified oligonucleotide and optionally a conjugate group. The oligonucleotide of the second oligomeric compound of such double-stranded antisense compound may be modified or unmodified. Either or both oligomeric compounds of a double-stranded antisense compound may comprise a conjugate group. The oligomeric compounds of double-stranded antisense compounds may include non-complementary overhanging nucleosides.

In certain embodiments, oligomeric compounds of antisense compounds are capable of hybridizing to a target nucleic acid, resulting in at least one antisense activity. In certain embodiments, antisense compounds selectively affect one or more target nucleic acid. Such selective antisense compounds comprises a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, the invention provides antisense compounds that are sufficiently “DNA-like” to elicit RNase H activity. Further, in certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain such embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain such embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

IV. Certain Target Nucleic Acids

In certain embodiments, antisense compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: an mRNA and a pre-mRNA, including intronic, exonic and untranslated regions. In certain embodiments, the target RNA is an mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron.

In certain embodiments, the target nucleic acid is a non-coding RNA. In certain such embodiments, the target non-coding RNA is selected from: a long-non-coding RNA, a short non-coding RNA, an intronic RNA molecule, a snoRNA, a scaRNA, a microRNA (including pre-microRNA and mature microRNA), a ribosomal RNA, and promoter directed RNA. In certain embodiments, the target nucleic acid is a nucleic acid other than a mature mRNA. In certain embodiments, the target nucleic acid is a nucleic acid other than a mature mRNA or a microRNA. In certain embodiments, the target nucleic acid is a non-coding RNA other than a microRNA. In certain embodiments, the target nucleic acid is a non-coding RNA other than a microRNA or an intronic region of a pre-mRNA. In certain embodiments, the target nucleic acid is a long non-coding RNA. In certain embodiments, the target nucleic acid is a non-coding RNA associated with splicing of other pre-mRNAs. In certain embodiments, the target nucleic acid is a nuclear-retained non-coding RNA.

In certain embodiments, antisense compounds described herein are complementary to a target nucleic acid comprising a single-nucleotide polymorphism (SNP). In certain such embodiments, the antisense compound is capable of modulating expression of one allele of the SNP-containing target nucleic acid to a greater or lesser extent than it modulates another allele. In certain embodiments, an antisense compound hybridizes to a (SNP)-containing target nucleic acid at the single-nucleotide polymorphism site.

In certain embodiments, antisense compounds are at least partially complementary to more than one target nucleic acid. For example, antisense compounds of the present invention may mimic microRNAs, which typically bind to multiple targets.

A. Complementarity/Mismatches to the Target Nucleic Acid

In certain embodiments, antisense compounds comprise antisense oligonucleotides that are complementary to the target nucleic acid over the entire length of the oligonucleotide. In certain embodiments, such oligonucleotides are 99% complementary to the target nucleic acid. In certain embodiments, such oligonucleotides are 95% complementary to the target nucleic acid. In certain embodiments, such oligonucleotides are 90% complementary to the target nucleic acid. In certain embodiments, such oligonucleotides are 85% complementary to the target nucleic acid. In certain embodiments, such oligonucleotides are 80% complementary to the target nucleic acid. In certain embodiments, antisense oligonucleotides are at least 80% complementary to the target nucleic acid over the entire length of the oligonucleotide and comprise a region that is 100% or fully complementary to a target nucleic acid. In certain such embodiments, the region of full complementarity is from 6 to 20 nucleobases in length. In certain such embodiments, the region of full complementarity is from 10 to 18 nucleobases in length. In certain such embodiments, the region of full complementarity is from 18 to 20 nucleobases in length.

In certain embodiments, the oligomeric compounds of antisense compounds comprise one or more mismatched nucleobases relative to the target nucleic acid. In certain such embodiments, antisense activity against the target is reduced by such mismatch, but activity against a non-target is reduced by a greater amount. Thus, in certain such embodiments selectivity of the antisense compound is improved. In certain embodiments, the mismatch is specifically positioned within an oligonucleotide having a gapmer motif. In certain such embodiments, the mismatch is at position 1, 2, 3, 4, 5, 6, 7, or 8 from the 5′-end of the gap region. In certain such embodiments, the mismatch is at position 9, 8, 7, 6, 5, 4, 3, 2, 1 from the 3′-end of the gap region. In certain such embodiments, the mismatch is at position 1, 2, 3, or 4 from the 5′-end of the wing region. In certain such embodiments, the mismatch is at position 4, 3, 2, or 1 from the 3′-end of the wing region.

B. Certain Target Nucleic Acids in Certain Tissues

In certain embodiments, antisense compounds comprise or consist of an oligonucleotide comprising a region that is complementary to a target nucleic acid, wherein the target nucleic acid is expressed in an extra-hepatic tissue. Extra-hepatic tissues include, but are not limited to: skeletal muscle, cardiac muscle, smooth muscle, adipose, white adipose, spleen, bone, intestine, adrenal, testes, ovary, pancreas, pituitary, prostate, skin, uterus, bladder, brain, glomerulus, distal tubular epithelium, breast, lung, heart, kidney, ganglion, frontal cortex, spinal cord, trigeminal ganglia, sciatic nerve, dorsal root ganglion, epididymal fat, diaphragm, pancreas, and colon.

In certain embodiments, it is desirable to modulate the amount or activity of the extra-hepatic nucleic acid target. For example, in certain embodiments, it is desirable to modulate the amount or activity of a nucleic acid target in a tissue selected from among: skeletal muscle, cardiac muscle, smooth muscle, adipose, white adipose, spleen, bone, intestine, adrenal, testes, ovary, pancreas, pituitary, prostate, skin, uterus, bladder, brain, glomerulus, distal tubular epithelium, breast, lung, heart, kidney, ganglion, frontal cortex, spinal cord, trigeminal ganglia, sciatic nerve, dorsal root ganglion, epididymal fat, diaphragm, pancreas, and colon.

In certain embodiments, a nucleic acid transcript expressed in one type of cell or tissue may cause a particular disease or condition, whereas the same nucleic acid transcript expressed in another type of tissue does not cause a particular disease or condition. For example, in certain embodiments, a nucleic acid transcript having particular mutation that is expressed in the heart may cause one or more symptoms associated with heart disease. However, the same nucleic acid transcript having the same mutation expressed in the liver does not cause any symptoms associated with heart disease.

Likewise, certain nucleic acid transcripts or nucleic acid targets may be highly expressed in one type of tissue, but not other types of tissues. For example, certain nucleic acid transcripts or nucleic acid targets may be highly expressed in a tissue selected from among: skeletal muscle, cardiac muscle, smooth muscle, adipose, white adipose, spleen, bone, intestine, adrenal, testes, ovary, pancreas, pituitary, prostate, skin, uterus, bladder, brain, glomerulus, distal tubular epithelium, breast, lung, heart, kidney, ganglion, frontal cortex, spinal cord, trigeminal ganglia, sciatic nerve, dorsal root ganglion, epididymal fat, diaphragm, pancreas, or colon, but not highly expressed in the liver. Conjugated oligomeric compounds described herein increase distribution into such tisses.

Certain nucleic acid transcripts or nucleic acid targets may also be differentially expressed in one type cell or tissue, but not other types of cells or tissues. For example, certain nucleic acid transcripts or nucleic acid targets may be expressed in hepatocytes, but expressed in higher quantities in heart cells, fibroblasts, cardiomyocytes, endothelial cells, or tumor cells. For example, certain nucleic acid transcripts or nucleic acid targets may be expressed in the liver, but expressed in higher quantities in skeletal muscle, cardiac muscle, smooth muscle, adipose, white adipose, spleen, bone, intestine, adrenal, testes, ovary, pancreas, pituitary, prostate, skin, uterus, bladder, brain, glomerulus, distal tubular epithelium, breast, lung, heart, kidney, ganglion, frontal cortex, spinal cord, trigeminal ganglia, sciatic nerve, dorsal root ganglion, epididymal fat, diaphragm, pancreas, or colon tissue.

C. Certain Modified Oligonucleotides

In certain embodiments, disclosed here in are modified oligonucleotides designed to target certain nucleic acid targets. Tables A to D below describe certain modified oligonucleotides targeted to certain nucleic acid transcripts. In Tables A to D below, subscript “s” represents a phosphorothioate internucleoside linkage, subscript “o” represents a phosphate internucleoside linkage, subscript “d” represents a 2′-deoxynucleoside, subscript “e” represents a 2′-MOE modified nucleoside, and subscript “k” represents a cEt modified nucleoside. In tables A and B below, superscript “m” before a C represents a 5-methylcysteine.

TABLE A
Certain Modified Oligonucleotides

| Target | Isis No. | Sequence (5′-3′) | Motif | SEQ ID NO: |
|---|---|---|---|---|
| CRP | 329993 | AGCATAGTTAACGAGCTCCC | 5-10-5 MOE | 14 |
| PTPB1B | 404173 | AATGGTTTATTCCATGGCCA | 5-10-5 MOE | 15 |
| GCCR | 426115 | GCAGCCATGGTGATCAGGAG | 5-10-5 MOE | 16 |
| GCGR | 449884 | GGTTCCCGAGGTGCCCA | 3-10-4 MOE | 17 |
| FGFR4 | 463588 | GCACACTCAGCAGGACCCCC | 5-10-5 MOE | 18 |
| GHr | 532401 | CCACCTTTGGGTGAATAGCA | 5-10-5 MOE | 19 |
| DGAT2 | 484137 | TGCCATTTAATGAGCTTCAC | 5-10-5 MOE | 20 |
| DMPK | 598769 | TCCCGAATGTCCGACA | Mixed wing | 21 |
| CFB | 696844 | ATCCCACGCCCCTGTCCAGC | 5-10-5 MOE | 22 |
| | | | GalNAc | |

TABLE B
Certain Modified Oligonucleotides

| | Isis | | SEQ ID |
|---|---|---|---|
| Target | No. | Motif (5′-3′) | NO. |
| CRP | 329993 | AₑₛGₑₛ^{m}CₑₛAₑₛTₑₛA_{ds}G_{ds}T_{ds}T_{ds}A_{ds}A_{ds}^{m}C_{ds}G_{ds}A_{ds}G_{ds}^{m}CₑₛTₑₛ^{m}Cₑₛ^{m}Cₑₛ^{m}Cₑ | 14 |
| PTPB1B | 404173 | AₑₛAₑₛTₑₛGₑₛGₑₛT_{ds}T_{ds}T_{ds}A_{ds}T_{ds}T_{ds}^{m}C_{ds}^{m}C_{ds}A_{ds}T_{ds}GₑₛGₑₛ^{m}Cₑₛ^{m}CₑₛAₑ | 15 |
| GCCR | 426115 | Gₑₛ^{m}CₑₛAₑₛGₑₛ^{m}Cₑₛ^{m}C_{ds}A_{ds}T_{ds}G_{ds}G_{ds}T_{ds}G_{ds}A_{ds}T_{ds}^{m}C_{ds}AₑₛGₑₛGₑₛAₑₛGₑ | 16 |
| GCGR | 449884 | GₑₛGₑₛTₑₛT_{ds}^{m}C_{ds}^{m}C_{ds}^{m}C_{ds}G_{ds}A_{ds}G_{ds}G_{ds}T_{ds}G_{ds}^{m}Cₑₛ^{m}Cₑₛ^{m}CₑₛAₑ | 17 |
| FGFR4 | 463588 | Gₑₛ^{m}CₑₛAₑₛ^{m}CₑₛAₑₛ^{m}C_{ds}T_{ds}^{m}C_{ds}A_{ds}G_{ds}^{m}C_{ds}A_{ds}G_{ds}G_{ds}A_{ds}^{m}Cₑₛ^{m}Cₑₛ^{m}Cₑₛ^{m}Cₑₛ^{m}Cₑ | 18 |
| GHr | 532401 | ^{m}Cₑₛ^{m}CₑₛAₑₛ^{m}Cₑₛ^{m}CₑₛT_{ds}T_{ds}T_{ds}G_{ds}G_{ds}G_{ds}T_{ds}G_{ds}A_{ds}A_{ds}TₑₛAₑₛGₑₛ^{m}CₑₛAₑ | 19 |
| DGAT2 | 484137 | TₑₛGₑₛ^{m}Cₑₛ^{m}CₑₛAₑₛT_{ds}T_{ds}T_{ds}A_{ds}A_{ds}T_{ds}G_{ds}A_{ds}G_{ds}^{m}C_{ds}TₑₛTₑₛ^{m}CₑₛAₑₛ^{m}Cₑ | 20 |
| DMPK | 598769 | Tₑₛ^{m}Cₑₛ^{m}Cₖₛ^{m}CₖₛG_{ds}A_{ds}A_{ds}T_{ds}G_{ds}T_{ds}^{m}C_{ds}^{m}C_{ds}GₖₛAₖₛ^{m}CₑₛAₑ | 21 |
| CFB | 696844 | AₑₛTₑₛ^{m}Cₑₛ^{m}Cₑₛ^{m}CₑₛA_{ds}^{m}C_{ds}G_{ds}^{m}C_{ds}^{m}C_{ds}^{m}C_{ds}^{m}C_{ds}T_{ds}G_{ds}T_{ds}^{m}Cₑₛ^{m}CₑₛAₑₛGₑₛ^{m}Cₑ | 22 |

TABLE C
Certain Modified Oligonucleotides Targeted to HBV

| Isis | | SEQ ID |
|---|---|---|
| No. | Motif (5′-3′) | NO. |
| 505358 | Ges mCes Aes Ges Aes Gds Gds Tds Gds Ads Ads Gds mCds Gds | 23 |
| | Ads Aes Ges Tes Ges mCe | |
| 509934 | mCes mCes Aes Aes Tes Tds Tds Ads Tds Gds mCds mCds Tds | 24 |
| | Ads mCds Aes Ges mCes mCes Te | |
| 510100 | Ges Ges mCes Ads Tds Ads Gds mCds Ads Gds mCds Ads Gds | 25 |
| | Ges Aes Tes Ge | |
| 552023 | Aes Ges Ges Aes Ges Tes Tds mCds mCds Gds mCds Ads Gds | 26 |
| | Tds Ads Tds Ges Ges Aes Te | |
| 552024 | Ges Tes Ges Aes Aes Ges mCds Gds Ads Ads Gds Tds Gds mCds | 27 |
| | Ads mCds Aes mCes Ges Ge | |
| 552032 | Ges Tes Ges mCes Aes Ges Ads Gds Gds Tds Gds Ads Ads Gds | 28 |
| | mCds Gds Aes Aes Ges Te | |
| 552859 | Aes Gks Gks Tds Gds Ads Ads Gds mCds Gds Ads Ads Gds Tks | 29 |
| | Gks mCe | |
| 552925 | Tes mCks mCds Gds mCds Ads Gds Tds Ads Tds Gds Gds Aks | 30 |
| | Tes mCks Ge | |
| 577119 | Aks Ads Tks Tds Tks Ads Tds Gds mCds mCds Tds Ads mCds | 31 |
| | Aes Ges mCes mCes Te | |

For table C above, A = an adenine, mC = a 5′-methylcytosine, G = a guanine, T = a thymine, e = a 2′-O-methoxyethyl modified nucleoside, k = a cEt modified nucleoside, d = a 2′-deoxynucleoside, and s = a phosphorothioate internucleoside linkage.

TABLE D
Certain Modified Oligonucleotides Targeted to TTR

| Isis | | SEQ ID |
|---|---|---|
| No. | Motif (5′-3′) | NO. |
| 420915 | Tes mCes Tes Tes Ges Gds Tds Tds Ads mCds Ads Tds Gds Ads | 32 |
| | Ads Aes Tes mCes mCes mCe | |
| 304299 | mCes Tes Tes Ges Ges Tds Tds Ads mCds Ads Tds Gds Ads Ads | 33 |
| | Ads Tes mCes mCes mCes Ae | |
| 420921 | Ges Ges Aes Aes Tes Ads mCds Tds mCds Tds Tds Gds Gds Tds | 34 |
| | Tds Aes mCes Aes Tes Ge | |
| 420922 | Tes Ges Ges Aes Aes Tds Ads mCds Tds mCds Tds Tds Gds Gds | 35 |
| | Tds Tes Aes mCes Aes Te | |
| 420950 | Tes Tes Tes Tes Aes Tds Tds Gds Tds mCds Tds mCds Tds Gds | 36 |
| | mCds mCes Tes Ges Ges Ae | |
| 420955 | Ges Aes Aes Tes Ges Tds Tds Tds Tds Ads Tds Tds Gds Tds | 37 |
| | mCds Tes mCes Tes Ges mCe | |
| 420957 | Aes Ges Ges Aes Aes Tds Gds Tds Tds Tds Tds Ads Tds Tds | 38 |
| | Gds Tes mCes Tes mCes Te | |
| 420959 | Aes mCes Aes Ges Ges Ads Ads Tds Gds Tds Tds Tds Tds Ads | 39 |
| | Tds Tes Ges Tes mCes Te | |

For table D above, A = an adenine, mC = a 5′-methylcytosine, G = a guanine, T = a thymine, e = a 2′-O-methoxyethyl modified nucleoside, k = a cEt modified nucleoside, d = a 2′-deoxynucleoside, and s = a phosphorothioate intemucleoside linkage.

V. Certain Pharmaceutical Compositions

In certain embodiments, the present invention provides pharmaceutical compositions comprising one or more antisense compound or a salt thereof. In certain such embodiments, the pharmaceutical composition comprises a suitable pharmaceutically acceptable diluent or carrier. In certain embodiments, a pharmaceutical composition comprises a sterile saline solution and one or more antisense compound. In certain embodiments, such pharmaceutical composition consists of a sterile saline solution and one or more antisense compound. In certain embodiments, the sterile saline is pharmaceutical grade saline. In certain embodiments, a pharmaceutical composition comprises one or more antisense compound and sterile water. In certain embodiments, a pharmaceutical composition consists of one antisense compound and sterile water. In certain embodiments, the sterile water is pharmaceutical grade water. In certain embodiments, a pharmaceutical composition comprises one or more antisense compound and phosphate-buffered saline (PBS). In certain embodiments, a pharmaceutical composition consists of one or more antisense compound and sterile PBS. In certain embodiments, the sterile PBS is pharmaceutical grade PBS.

In certain embodiments, pharmaceutical compositions comprise one or more or antisense compound and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, antisense compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an antisense compound encompass any pharmaceutically acceptable salts of the antisense compound, esters of the antisense compound, or salts of such esters. In certain embodiments, pharmaceutical compositions comprising antisense compounds comprising one or more antisense oligonucleotide, upon administration to an animal, including a human, are capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an antisense compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain.

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Included in the compounds provided herein are all such possible isomers, including their racemic and optically pure forms, unless specified otherwise. Likewise, all cis- and trans-isomers and tautomeric forms are also included unless otherwise indicated. Unless otherwise indicated, compounds described herein are intended to include corresponding salt forms.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Effects of Oligomeric Compounds Comprising a Lipophilic Conjugate Group In Vitro

The oligomeric compounds described in the table below are complementary to both human and mouse Metastasis Associated Lung Adenocarcinoma Transcript 1 (MALAT-1) transcript. Their effects on MALAT-1 expression were tested in vitro. Primary mouse hepatocytes were isolated from wild type BALB/c mice and plated at a density of 35,000 cells per well Immediately after the cells were plated, the oligomeric compounds were added to the hepatocytes at the concentrations listed in the table below, and the cells were incubated overnight. No transfection reagents were used. The next day, the cells were lysed in Buffer RLT and RNA extracted using RNeasy (Qiagen, Germantown, Md.). MALAT-1 RNA levels were measured using RT-qPCR and normalized to total RNA using RiboGreen (Thermo Fisher Scientific, Carlsbad, Calif.). The results are shown below as the percent normalized MALAT-1 RNA levels relative to untreated control cells (% UTC) for each concentration of oligomeric compound tested, and the calculated half maximal inhibitory concentrations (IC₅₀) are shown.

TABLE 1
MALAT-1 expression in vitro

| | | Concentration | MALAT-1 | IC₅₀ | SEQ |
|---|---|---|---|---|---|
| Isis No. | Sequence (5′ to 3′) | (nM) | level (% UTC) | (nM) | ID NO. |
| 626112 | Gₑₛ ^{m}Cₑₒ ^{m}Cₑₒ Aₑₒ Gₑₒ G_{ds} ^{m}C_{ds} | 0.032 | 108 | 16 | 1 |
| | T_{ds} G_{ds} G_{ds} T_{ds} T_{ds} A_{ds} T_{ds} G_{ds} | 0.16 | 105 | | |
| | Aₑₒ ^{m}Cₑₒ Tₑₛ ^{m}Cₑₛ Aₑ | 0.80 | 121 | | |
| | | 4.0 | 93 | | |
| | | 20.0 | 41 | | |
| | | 100.0 | 6 | | |
| 724784 | Chol-TEG-T_{do} ^{m}C_{do} A_{do} Gₑₛ ^{m}Cₑₛ | 0.032 | 122 | 18 | 2 |
| | ^{m}Cₑₛ Aₑₛ GₑₛG_{ds} ^{m}C_{ds} T_{ds} G_{ds} G_{ds} T_{ds} | 0.16 | 112 | | |
| | T_{ds} A_{ds} T_{ds} G_{ds} Aₑₛ ^{m}Cₑₛ Tₑₛ ^{m}Cₑₛ Aₑ | 0.80 | 92 | | |
| | | 4.0 | 94 | | |
| | | 20.0 | 48 | | |
| | | 100.0 | 15 | | |

Subscripts in the table above: “s” represents a phosphorothioate internucleoside linkage, “o” represents a phosphate internucleoside linkage, “d” represents a 2′-deoxynucleoside, “e” represents a 2′-MOE modified nucleoside.
Superscripts: “m” before a C represents a 5-methylcysteine.
The structure of “Chol-TEG-”, is shown below:

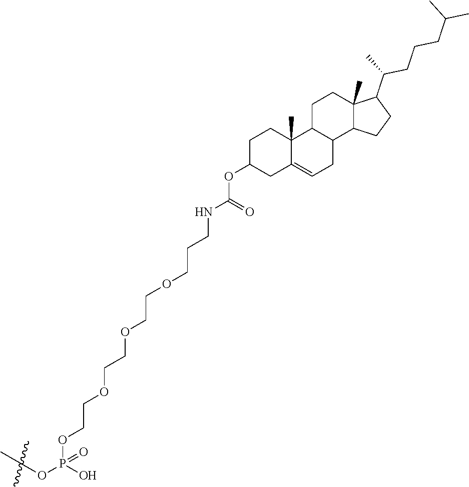 [SMILES: *OP(=O)(O)OCCOCCOCCOCCCNC(=O)OC1CC[C@@]2(C)C(=CCC3C4CCC([C@H](C)CCCC(C)C)[C@@]4(C)CCC32)C1]

### Example 2: Effects of Oligomeric Compounds Comprising a Lipophilic Conjugate Group In Vivo

The oligomeric compounds described in the table below are complementary to both human and mouse MALAT-1 transcripts. Their effects on MALAT-1 expression were tested in vivo. Wild type C57bl/6 mice each received a 100 mg/kg intravenous injection, via the tail vein, of an oligomeric compound listed in the table below or saline vehicle alone. Each treatment group consisted of four mice. Eight days after the injection, the animals were sacrificed. MALAT-1 RNA expression was analyzed in liver, kidney, lung, ganglion, frontal cortex, and spinal cord by RT-qPCR and normalized to total RNA using RiboGreen (Thermo Fisher Scientific, Carlsbad, Calif.). The average results for each group are shown below as the percent normalized MALAT-1 RNA levels relative to average results for the vehicle treated animals

TABLE 2
MALAT-1 expression in vivo

| | | MALAT-1 RNA level (% Vehicle) | | | | | | SEQ |
|---|---|---|---|---|---|---|---|---|
| Isis | | | | Gangl | Fr. | Sp. | | ID |
| No. | Sequence (5′ to 3′) | Liver | Kidney | Lung | ion | Cor. | Cord | NO. |
| 626112 | Gₑₛ^{m}Cₑₒ^{m}CₑₒAₑₒGₑₒG_{ds}^{m}C_{ds}T_{ds} | 16 | 46 | 65 | 47 | 88 | 89 | 1 |
| | G_{ds}G_{ds}T_{ds}T_{ds}A_{ds}T_{ds}G_{ds}Aₑₒ^{m}Cₑₒ | | | | | | | |
| | Tₑₛ^{m}CₑₛAₑ | | | | | | | |
| 724781 | Toco-TEG-T_{do}^{m}C_{do}A_{do}Gₑₛ^{m}Cₑₛ | 9.6 | 34 | 46 | 39 | 92 | 87 | 2 |
| | ^{m}CₑₛAₑₛGₑₛG_{ds}^{m}C_{ds}T_{ds}G_{ds}G_{ds}T_{ds} | | | | | | | |
| | T_{ds}A_{ds}T_{ds}G_{ds}Aₑₛ^{m}CₑₛTₑₛ^{m}CₑₛAₑ | | | | | | | |
| 724782 | C10-TEG-T_{do}^{m}C_{do}A_{do}Gₑₛ^{m}Cₑₛ | 8.8 | 24 | 44 | 21 | 95 | 69 | 2 |
| | ^{m}CₑₛAₑₛGₑₛG_{ds}^{m}C_{ds}T_{ds}G_{ds}G_{ds}T_{ds} | | | | | | | |
| | T_{ds}A_{ds}T_{ds}G_{ds}Aₑₛ^{m}CₑₛTₑₛ^{m}CₑₛAₑ | | | | | | | |
| 724783 | C16-TEG-T_{do}^{m}C_{do}A_{do}Gₑₛ^{m}Cₑₛ | 7.7 | 39 | 39 | 19 | 93 | 70 | 2 |
| | ^{m}CₑₛAₑₛGₑₛG_{ds}^{m}C_{ds}T_{ds}G_{ds}G_{ds}T_{ds} | | | | | | | |
| | T_{ds}A_{ds}T_{ds}G_{ds}Aₑₛ^{m}CₑₛTₑₛ^{m}CₑₛAₑ | | | | | | | |
| 724784 | Chol-TEG-T_{do}^{m}C_{do}A_{do}Gₑₛ^{m}Cₑₛ | 6.8 | 24 | 23 | 28 | 105 | 99 | 2 |
| | ^{m}CₑₛAₑₛGₑₛG_{ds}^{m}C_{ds}T_{ds}G_{ds}G_{ds}T_{ds} | | | | | | | |
| | T_{ds}A_{ds}T_{ds}G_{ds}Aₑₛ^{m}CₑₛTₑₛ^{m}CₑₛAₑ | | | | | | | |

See legend for Table 1 for subscripts and superscript key. The structure of “Chol-TEG-”, is shown in Example 1. The structure of “Toco-TEG-” is:

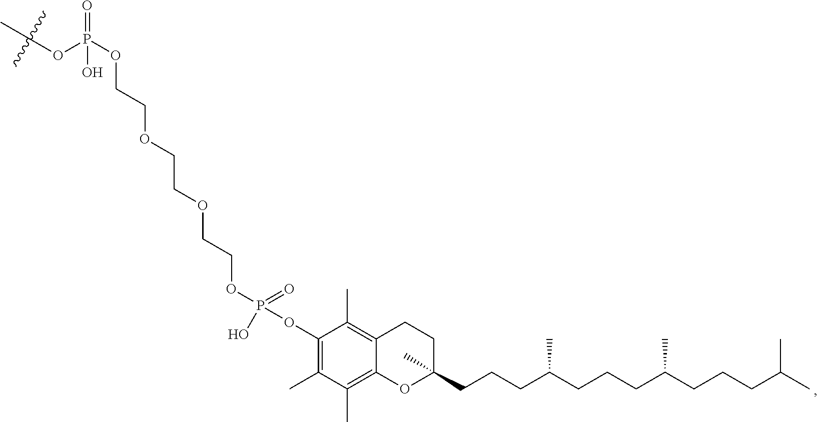

the structures of “C10-TEG” and “C16-TEG-” are:

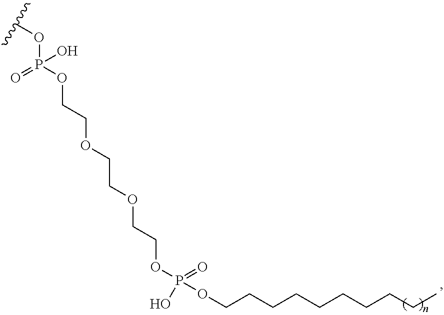

wherein n is 1 in “C10-TEG-”, and n is 7 in “C16-TEG-”.

### Example 3: Effects of Oligomeric Compounds Comprising a Lipophilic Conjugate Group In Vivo

The oligomeric compounds described in the table below are complementary to both human and mouse MALAT-1 transcripts. Their effects on MALAT-1 expression were tested in vivo. Wild type C57bl/6 mice each received an intravenous injection, via the tail vein, of 4.5 μmol/kg of an oligomeric compound listed in the table below or saline vehicle alone. Each treatment group consisted of three or four mice. Three days after the injection, the animals were sacrificed. MALAT-1 RNA expression was analyzed in heart, macrophages (Macs), trigeminal ganglia (TG), sciatic nerve (SN), and dorsal root ganglion (DRG) by RT-qPCR and normalized to total RNA using RiboGreen (Thermo Fisher Scientific, Carlsbad, Calif.). The average results for each group are shown below as the percent normalized MALAT-1 RNA levels relative to average results for the vehicle treated animals.

TABLE 3
MALAT-1 expression in vivo

| Isis | | MALAT-1 RNA level (% Vehicle) | | | | | SEQ ID |
|---|---|---|---|---|---|---|---|
| No. | Sequence (5′ to 3′) | Heart | Macs | TG | SN | DRG | NO. |
| 556089 | Gₖₛ^{m}CₖₛAₖₛT_{ds}T_{ds}^{m}C_{ds}T_{ds}A_{ds}A_{ds}T_{ds}A_{ds} | 67 | 48 | 79 | 77 | 88 | 3 |
| | G_{ds}^{m}C_{ds}AₖₛGₖₛ^{m}Cₖ | | | | | | |
| 827935 | Toco-TEG-T_{do}^{m}C_{do}A_{do}GₖₛCₖₛAₖₛT_{ds} | 69 | 38 | 66 | 62 | 87 | 4 |
| | T_{ds}^{m}C_{ds}T_{ds}A_{ds}A_{ds}T_{ds}A_{ds}G_{ds}^{m}C_{ds}Aₖₛ | | | | | | |
| | Gₖₛ^{m}Cₖ | | | | | | |
| 812134 | C16-HA-T_{do}^{m}C_{do}A_{do}Gₖₛ^{m}CₖₛAₖₛT_{ds} | 28 | 37 | 58 | 64 | 59 | 4 |
| | T_{ds}^{m}C_{ds}T_{ds}A_{ds}A_{ds}T_{ds}A_{ds}G_{ds}^{m}C_{ds}Aₖₛ | | | | | | |
| | Gₖₛ^{m}Cₖ | | | | | | |

See legend for Table 1 for subscripts and superscript key. The structure of “Toco-TEG-”, is shown in Example 2. The structure of “C16-HA” is:

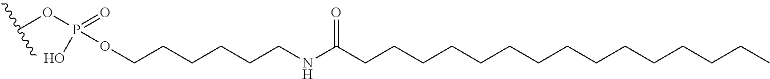 [SMILES: *OP(=O)(O)OCCCCCCNC(=O)CCCCCCCCCCCCCCC]

### Example 4: Dose Response Effects of Oligomeric Compounds Comprising a Lipophilic Conjugate Group In Vivo

The oligomeric compounds described in the table below are complementary to both human and mouse MALAT-1 transcripts. Their effects on MALAT-1 expression were tested in vivo. Male diet-induced obesity (DIO) mice each received an intravenous injection, via the tail vein, of an oligomeric compound listed in the table below or saline vehicle alone once per week for two weeks. Each treatment group consisted of three or four mice. Three days after the final injection, the animals were sacrificed. MALAT-1 RNA expression was analyzed in liver, heart, lung, white adipose tissue (WAT), and brown adipose tissue (BAT) by RT-qPCR and normalized to total RNA using RiboGreen (Thermo Fisher Scientific, Carlsbad, Calif.). The average results for each group are shown below as the percent normalized MALAT-1 RNA levels relative to average results for the vehicle treated animals.

TABLE 4
MALAT-1 expression in vivo

| Isis | | Dosage | MALAT-1 RNA level (% Vehicle) | | | | | SEQ |
|---|---|---|---|---|---|---|---|---|
| No. | Sequence (5′ to 3′) | (μmol/kg/week) | Liver | Heart | WAT | Lung | BAT | ID NO. |
| 556089 | Gₖₛ^{m}CₖₛAₖₛT_{ds}T_{ds}^{m}C_{ds}T_{ds}A_{ds} | 0.2 | 51 | 105 | 88 | 88 | 79 | 3 |
| | A_{ds}T_{ds}A_{ds}G_{ds}^{m}C_{ds}AₖₛGₖₛ^{m}Cₖ | 0.6 | 20 | 104 | 61 | 72 | 63 | |
| | | 1.8 | 6 | 74 | 31 | 49 | 35 | |
| 812133 | Ole-HA-T_{do}^{m}C_{do}A_{do}Gₖₛ^{m}Cₖₛ | 0.2 | 25 | 71 | 53 | 81 | 56 | 4 |
| | AₖₛT_{ds}T_{ds}^{m}C_{ds}T_{ds}A_{ds}A_{ds}T_{ds} | 0.6 | 10 | 61 | 39 | 59 | 38 | |
| | A_{ds}G_{ds}^{m}C_{ds}AₖₛGₖₛ^{m}Cₖ | 1.8 | 5 | 42 | 23 | 40 | 23 | |
| 812134 | C16-HA-T_{do}^{m}C_{do}A_{do}Gₖₛ^{m}Cₖₛ | 0.2 | 23 | 86 | 55 | 101 | 84 | 4 |
| | AₖₛT_{ds}T_{ds}^{m}C_{ds}T_{ds}A_{ds}A_{ds}T_{ds} | 0.6 | 13 | 65 | 35 | 100 | 46 | |
| | A_{ds}G_{ds}^{m}C_{ds}AₖₛGₖₛ^{m}Cₖ | 1.8 | 4 | 31 | 25 | 30 | 15 | |

See legend for Table 1 for subscripts and superscript key. Subscript “k” represents a cEt modified bicyclic sugar moiety. The structure of “C16-HA-”, is shown in Example 3. The structure of “Ole-HA-” is:

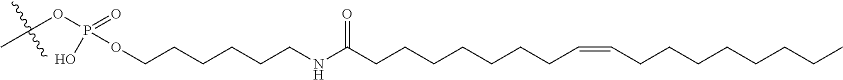 [SMILES: *OP(=O)(O)OCCCCCCNC(=O)CCCCCCC/C=C\CCCCCCCC]

### Example 5: Effects of Oligomeric Compounds Comprising a Lipophilic Conjugate Group In Vivo Following Different Routes of Administration

The effects of Isis Numbers 556089 and 812134 (see Example 4) on MALAT-1 expression were tested in vivo. Male, wild type C57bl/6 mice each received either an intravenous (IV) injection, via the tail vein, or a subcutaneous (SC) injection of Isis No. 556089, Isis No. 812134, or saline vehicle alone. Each treatment group consisted of four mice. Three days after the injection, the animals were sacrificed. MALAT-1 RNA expression was analyzed in liver, heart, and white adipose tissue (WAT) by RT-qPCR and normalized to total RNA using RiboGreen (Thermo Fisher Scientific, Carlsbad, Calif.). The average results for each group are shown below as the percent normalized MALAT-1 RNA levels relative to average results for the vehicle treated animals.

TABLE 5
MALAT-1 expression in vivo

| | | | MALAT-1 RNA level | | | SEQ |
|---|---|---|---|---|---|---|
| | Dosage | Route of | (% Vehicle) | | | ID |
| Isis No. | (mol/kg) | administration | Liver | Heart | WAT | NO. |
| 556089 | 0.4 | SC | 30 | 85 | 70 | 3 |
| | 1.2 | SC | 21 | 79 | 60 | |
| | 3.6 | SC | 13 | 53 | 37 | |
| | | IV | 12 | 56 | 32 | |
| 812134 | 0.4 | SC | 37 | 71 | 62 | 4 |
| | 1.2 | SC | 16 | 48 | 47 | |
| | 3.6 | SC | 8 | 29 | 25 | |
| | | IV | 5 | 30 | 18 | |

### Example 6: Effects of Oligomeric Compounds Comprising a Lipophilic Conjugate Group In Vivo in a Cancer Model

The effects of Isis Numbers 556089 and 812134 (see Example 4) on MALAT-1 expression were tested in vivo in human epidermoid A431 tumor-bearing mice. Female, NCr-Nude mice were subcutaneously injected in the left flank with 10 million A431 cells. Once the tumors reached approximately 100 mm³ in size, each mouse received a subcutaneous injection of 50 mg/kg of Isis No. 556089 (9.23 μmol/kg) or Isis No. 812134 (7.1 μmol/kg), or saline vehicle alone. Each treatment group consisted of three mice. Twenty-four hours after the injection, the animals were sacrificed. MALAT-1 RNA expression was analyzed in a tumor, liver, kidney, heart, lung, fat, and muscle by RT-qPCR, with species-specific primer/probe sets, and normalized to mouse cyclophilin or human beta-actin levels. The average results for each group are shown below as the percent normalized MALAT-1 RNA levels relative to average results for the vehicle treated animals

TABLE 6
MALAT-1 expression in vivo

| | | | | | | | | Human MALAT-1 |
|---|---|---|---|---|---|---|---|---|
| | Mouse MALAT-1 RNA level (% Vehicle) | | | | | | | RNA in tumor |
| Isis No. | Tumor | Liver | Kidney | Heart | Lung | Fat | Muscle | (% Vehicle) |
| 556089 | 38 | 17 | 65 | 107 | 56 | 40 | 113 | 47 |
| 812134 | 26 | 7 | 44 | 65 | 44 | 13 | 67 | 45 |

### Example 7: Effects of Oligomeric Compounds Comprising a Lipophilic Conjugate Group In Vivo

The oligomeric compounds described in the table below are complementary to mouse CD36 transcript. Their effects on CD36 expression were tested in vivo. Wild type C57bl/6 mice each received an intravenous injection of an oligomeric compound at a dosage listed in the table below or saline vehicle alone. Each treatment group consisted of three mice. Three days after the injection, the animals were sacrificed. CD36 mRNA expression was analyzed in liver, kidney, heart, quadriceps, and epididymal fat by RT-qPCR and normalized to total RNA using RiboGreen (Thermo Fisher Scientific, Carlsbad, Calif.). The average results for each group are shown below as the percent normalized CD36 RNA levels relative to average results for the vehicle treated animals. Tissue distribution of the oligomeric compounds was analyzed using HPLC-MS detection of the parent oligonucleotide (Isis No. 583363), which is generated from Isis No. 847939 following cleavage of the linker nucleosides and conjugate group. The results are shown below as the average parent oligonucleotide mass per unit of tissue mass for each treatment group.

TABLE 7
CD36 expression in vivo

| | Sequence | Dosage | C36 mRNA level (% Vehicle) | | | | | |
|---|---|---|---|---|---|---|---|---|
| Isis No. | (5′ to 3′) | (mol/kg) | Liver | Kidney | Heart | Quad | Fat | SEQ ID NO. |
| 583363 | AₖₛGₖₛGₖₛA_{ds}T_{ds}A_{ds}T_{ds}G_{ds} | 1.7 | 52 | 87 | 95 | 107 | 100 | 5 |
| | G_{ds}A_{ds}A_{ds}^{m}C_{ds}^{m}C_{ds}AₖₛAₖₛAₖ | 5 | 35 | 42 | 85 | 133 | 91 | |
| | | 15 | 15 | 42 | 84 | 62 | 82 | |
| 847939 | c16-HA-T_{do}^{m}C_{do}A_{do}AₖₛGₖₛ | 1.7 | 31 | 90 | 89 | 82 | 87 | 6 |
| | GₖₛA_{ds}T_{ds}A_{ds}T_{ds}G_{ds}G_{ds}A_{ds} | 5 | 7 | 44 | 65 | 60 | 40 | |
| | A_{ds}^{m}C_{ds}^{m}C_{ds}AₖₛAₖₛAₖ | 15 | 3 | 23 | 18 | 34 | 12 | |

See legend for Tables 1 and 4 for subscripts and superscript key. The structure of “C16-HA” is shown in Example 3.

TABLE 8
CD36 tissue distribution

| | Dosage | Mass of 583363/Tissue mass (μg/g) | | | | |
|---|---|---|---|---|---|---|
| Isis No. | (μmol/kg) | Liver | Kidney | Heart | Quad | Fat |
| 583363 | 1.7 | 5 | 31 | 1 | <1 | <1 |
| | 5 | 10 | 58 | 1 | 1 | <1 |
| | 15 | 29 | 128 | 2 | 2 | 1 |
| 847939 | 1.7 | 16 | 29 | 1 | <1 | <1 |
| | 5 | 47 | 95 | 5 | 2 | 2 |
| | 15 | 257 | 218 | 18 | 8 | 29 |

### Example 8: Effects of Oligomeric Compounds Comprising a Lipophilic Conjugate Group In Vivo Following Different Routes of Administration

The effects of Isis Numbers 583363 and 847939 (see Example 7) on CD36 expression were tested in vivo. Female, wild type C57bl/6 mice each received either an intravenous injection or an intraperitoneal injection of Isis No. 583363, Isis No. 847939, or saline vehicle alone once per week for three weeks. Each treatment group consisted of four mice. Three days after the final injection, the animals were sacrificed. CD36 mRNA expression was analyzed in liver, kidney, heart, lung, quadriceps, fat, and peritoneal macrophages (Macs) by RT-qPCR and normalized to total RNA using RiboGreen (Thermo Fisher Scientific, Carlsbad, Calif.). The average results for each group are shown below as the percent normalized CD36 RNA levels relative to average results for the vehicle treated animals

TABLE 9
CD36 expression in vivo

| | Dosage | | | | | | | | |
|---|---|---|---|---|---|---|---|---|---|
| | (μmol | | | | | | | | |
| | /kg/ | Route of | CD36 mRNA level (% Vehicle) | | | | | | |
| Isis No. | week) | administration | Liver | Kidney | Lung | Heart | Quad | Fat | Macs |
| 583363 | 1 | IV | 56 | 80 | 85 | 102 | 84 | 95 | 96 |
| | 3 | IV | 24 | 37 | 84 | 98 | 69 | 74 | 80 |
| | 9 | IV | 11 | 20 | 81 | 81 | 30 | 46 | 51 |
| | | IP | 15 | 7 | 82 | 94 | 36 | 28 | 28 |
| 847939 | 1 | IV | 22 | 78 | 90 | 94 | 37 | 62 | 98 |
| | 3 | IV | 12 | 33 | 66 | 69 | 22 | 31 | 56 |
| | 9 | IV | 11 | 3 | 45 | 28 | 9 | 7 | 19 |
| | | IP | 18 | 7 | 56 | 52 | 21 | 10 | 29 |

### Example 9: Effects of Oligomeric Compounds Comprising a Lipophilic Conjugate Group In Vivo

The oligomeric compounds described in the table below are complementary to mouse adipose triglyceride lipase (ATGL) transcript. Their effects on ATGL expression were tested in vivo. Male, DIO mice each received an intravenous injection of an oligomeric compound at a dosage listed in the table below or saline vehicle alone. Each treatment group consisted of three mice, except for the high dose 829311 group, which consisted of two mice. Three days after the injection, the animals were sacrificed. CD36 mRNA expression was analyzed in liver, heart, and epididymal fat by RT-qPCR and normalized to total RNA using RiboGreen (Thermo Fisher Scientific, Carlsbad, Calif.). The average results for each group are shown below as the percent normalized ATGL RNA levels relative to average results for the vehicle treated animals

TABLE 10
ATGL expression in vivo

| Isis | | Dosage | CD36 mRNA level (% Vehicle) | | | SEQ ID |
|---|---|---|---|---|---|---|
| No. | Sequence (5′ to 3′) | (μmol/kg) | Liver | Heart | Fat | NO. |
| 606890 | GₖₛAₖₛ^{m}CₖₛA_{ds}A_{ds}^{m}C_{ds}T_{ds}T_{ds} | 1.8 | 61 | 105 | 63 | 7 |
| | G_{ds}G_{ds}A_{ds}G_{ds}^{m}C_{ds}TₖₛTₖₛGₖ | 3 | 61 | 95 | 94 | |
| | | 15 | 22 | 90 | 54 | |
| 829311 | C16-HA-T_{do}^{m}C_{do}A_{do}GₖₛAₖₛ^{m}Cₖₛ | 1.8 | 68 | 113 | 106 | 8 |
| | A_{ds}A_{ds}^{m}C_{ds}T_{ds}TsG_{ds}G_{ds}A_{ds}G_{ds} | 3 | 32 | 94 | 56 | |
| | ^{m}C_{ds}TₖₛTₖₛGₖ | 15 | 16 | 58 | 19 | |
| 829312 | Ole-HA-T_{do}^{m}C_{do}A_{do}GₖₛAₖₛ^{m}Cₖₛ | 1.8 | 64 | 101 | 84 | 8 |
| | A_{ds}A_{ds}^{m}C_{ds}T_{ds}T_{ds}G_{ds}G_{ds}A_{ds}G_{ds} | 3 | 26 | 62 | 53 | |
| | ^{m}C_{dsTks}TₖₛGₖ | 15 | 12 | 59 | 26 | |
| 829316 | Lin-HA-T_{do}^{m}C_{do}A_{do}GₖₛAₖₛ^{m}Cₖₛ | 3 | 32 | 90 | 52 | 8 |
| | A_{ds}A_{ds}^{m}C_{ds}T_{ds}T_{ds}G_{ds}G_{ds}A_{ds}G_{ds} | | | | | |
| | ^{m}C_{ds}TₖₛTₖₛGₖ | | | | | |

See legend for Tables 1 and 4 for subscripts and superscript key. The structures of “C16-HA” and “Ole-HA-” are shown in Examples 3 and 4, respectively. The structure or “Lin-HA-” is:

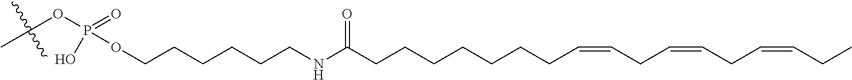 [SMILES: *OP(=O)(O)OCCCCCCNC(=O)CCCCCCC/C=C\C/C=C\C/C=C\CC]

### Example 10: Effects of Oligomeric Compounds Comprising a Lipophilic Conjugate Group In Vivo

The oligomeric compounds described in the table below are complementary to mouse eukaryotic translation initiation factor 4E binding protein 1 (eIF4E-BP1) transcript. Their effects on eIF4E-BP1 expression were tested in vivo. Female, wild type C57bl/6 mice each received an intravenous injection of an oligomeric compound listed in the table below or saline vehicle alone once per week for three weeks. Each treatment group consisted of three mice. Two days after the final injection, the animals were sacrificed. eIF4E-BP1 mRNA expression was analyzed in liver, kidney, heart, lung, muscle, fat, and colon by RT-qPCR and normalized to total RNA using RiboGreen (Thermo Fisher Scientific, Carlsbad, Calif.). The average results for each group are shown below as the percent normalized CD36 RNA levels relative to average results for the vehicle treated animals.

TABLE 11
eIF4E-BP1 expression in vivo

| Isis | | Dosage | eIF4E-BP1 mRNA level (% Vehicle) | | | | | | | SEQ ID |
|---|---|---|---|---|---|---|---|---|---|---|
| No. | Sequence | (μmol/kg/wk) | Liver | Kid | Lung | Heart | Musc | Fat | Col | NO. |
| 543226 | ^{m}CₑₛTₑₛGₖₛG_{ds} | 1.7 | 96 | 92 | 98 | 114 | 116 | 92 | 104 | 9 |
| | T_{ds}A_{ds}T_{ds}G_{ds}A_{ds} | 5 | 72 | 90 | 96 | 99 | 96 | 86 | 93 | |
| | G_{ds}G_{ds}^{m}C_{ds}^{m}C_{ds} | 15 | 57 | 99 | 84 | 109 | 88 | 74 | 85 | |
| | TₖₛGₖₛAₑ | | | | | | | | | |
| 835315 | C16-HA-T_{do}^{m}C_{do} | 1.7 | 82 | 82 | 97 | 100 | 84 | 72 | 96 | 10 |
| | A_{do}^{m}CₑₛTₑₛGₖₛ | 5 | 52 | 110 | 90 | 87 | 74 | 51 | 99 | |
| | G_{ds}T_{ds}A_{ds}T_{ds}G_{ds} | 15 | 46 | 95 | 80 | 63 | 59 | 25 | 79 | |
| | A_{ds}G_{ds}G_{ds}^{m}C_{ds} | | | | | | | | | |
| | ^{m}C_{ds}TₖₛGₖₛAₑ | | | | | | | | | |

See legend for Tables 1 and 4 for subscripts and superscript key. The structure of “C16-HA” is shown in Example 3.

### Example 11: Effects of Oligomeric Compounds Comprising a Lipophilic Conjugate Group In Vivo

The oligomeric compounds described in the table below are complementary to both human and mouse Dystrophia Myotonica-Protein Kinase (DMPK) transcript. Their effects on DMPK expression were tested in vivo. Wild type Balb/c mice each received an intravenous injection of an oligomeric compound at a dosage listed in the table below or saline vehicle alone. Each animal received one dose per week for 3½ weeks, for a total of 4 doses. Each treatment group consisted of three or four mice. Two days after the last dose, the animals were sacrificed. DMPK mRNA expression was analyzed in liver, kidney, and quadriceps by RT-qPCR and normalized to total RNA using RiboGreen (Thermo Fisher Scientific, Carlsbad, Calif.). The average results for each group are shown below as the percent normalized DMPK RNA levels relative to average results for the vehicle treated animals. An entry of “nd” means no data. The data below show that the oligomeric compounds comprising a lipophilic conjugate group were more potent in the quadriceps compared to the parent oligomeric compound that does not comprise a lipophilic conjugate group.

TABLE 12
DMPK expression in vivo

| Isis | | Dosage | DMPK mRNA level (% Vehicle) | | | SEQ ID |
|---|---|---|---|---|---|---|
| No. | Sequence (5′ to 3′) | (mg/kg/week) | Liver | Heart | Fat | NO. |
| 486178 | Aₖₛ^{m}CₖₛAₖₛA_{ds}T_{ds}A_{ds}A_{ds}A_{ds}T_{ds}A_{ds}^{m}C_{ds} | 12.5 | 19 | 77 | 50 | 11 |
| | ^{m}C_{ds}G_{ds}AₖₛGₖₛGₖ | 25 | 19 | 74 | 33 | |
| | | 50 | 15 | 64 | 14 | |
| 819733 | Chol-TEG-T_{ds}^{m}C_{do}A_{do}Aₖₛ^{m}CₖₛAₖₛA_{ds}T_{ds} | 12.5 | 19 | 78 | 8 | 12 |
| | A_{ds}A_{ds}A_{ds}T_{ds}A_{ds}^{m}C_{ds}^{m}C_{ds}G_{ds}AₖₛGₖₛGₖ | 25 | nd | nd | nd | |
| | | 50 | nd | nd | nd | |
| 819734 | Toco-TEG-T_{ds}^{m}C_{do}A_{do}Aₖₛ^{m}CₖₛAₖₛA_{ds}T_{ds} | 12.5 | 17 | 66 | 15 | 12 |
| | A_{ds}A_{ds}A_{ds}T_{ds}A_{ds}^{m}C_{ds}^{m}C_{ds}G_{ds}AₖₛGₖₛGₖ | 25 | 18 | 58 | 10 | |
| | Gₖₛ^{m}Cₖ | 50 | 17 | 58 | 5 | |

See legend for Tables 1 and 4 for subscripts and superscript key. The structures of “Choi-TEG-” and “Toco-TEG-” are shown in Examples 1 and 2, respectively.

### Example 12: Effects of Oligomeric Compounds Comprising a Lipophilic Conjugate Group In Vivo

The oligomeric compounds described in the table below are complementary to mouse PTEN transcript. Their effects on PTEN expression were tested in vivo. Wild type Balb/c mice each received a subcutaneous injection of an oligomeric compound at a dosage listed in the table below or saline vehicle alone. Each animal received two doses per week for 3 weeks except for the high dose group for Isis No. 449516, which received one dose per week. Each treatment group consisted of three or four mice. Two days after the last dose, the animals were sacrificed. PTEN mRNA expression was analyzed in liver, heart, diaphragm, tibialis anterior (TA), quadriceps, and gastrocnemius by RT-qPCR and normalized to total RNA using RiboGreen (Thermo Fisher Scientific, Carlsbad, Calif.). The average results for each group are shown below as the percent normalized PTEN mRNA levels relative to average results for the vehicle treated animals. The data below show that the oligomeric compounds comprising a lipophilic conjugate group without a readily cleavable moiety, such as a phosphate group, did not have improved potency in tissues other than the liver compared to the parent oligomeric compound that does not comprise a lipophilic conjugate group.

TABLE 13
PTEN expression in vivo

| | | Dosage | PTEN mRNA level (% Vehicle) | | | | | | SEQ |
|---|---|---|---|---|---|---|---|---|---|
| Isis No. | Sequence (5′ to 3′) | (mg/kg) | Liver | Heart | Diaph | TA | Quad | Gast | ID NO. |
| 392749 | Cₖₛ Uₖₛ T_{ds} A_{ds} G_{ds} C_{ds} | 1.25 | 77 | 129 | 138 | 140 | 167 | 83 | 13 |
| | A_{ds} C_{ds} T_{ds} G_{ds} G_{ds} C_{ds} | 3.75 | 34 | 133 | 133 | 129 | 99 | 120 | |
| | Cₖₛ | 12.5 | 7 | 78 | 66 | 78 | 51 | 61 | |
| 449514 | Cₖₛ Uₖₛ T_{ds} A_{ds} G_{ds} C_{ds} | 1.25 | 108 | 109 | 152 | 114 | 144 | 76 | 13 |
| | A_{ds} C_{ds} T_{ds} G_{ds} G_{ds} C_{ds} | 3.75 | 93 | 127 | 164 | 92 | 101 | 119 | |
| | Cₖₛ U_{HA-Chol} | 12.5 | 62 | 129 | 74 | 97 | 56 | 98 | |
| 449515 | Cₖₛ Uₖₛ T_{ds} A_{ds} G_{ds} C_{ds} | 1.25 | 62 | 184 | 107 | 99 | 151 | 98 | 13 |
| | A_{ds} C_{ds} T_{ds} G_{ds} G_{ds} C_{ds} | 3.75 | 19 | 110 | 109 | 97 | 97 | 96 | |
| | Cₖₛ U_{HA-C10} | 12.5 | 9 | 89 | 49 | 64 | 29 | 51 | |
| 449516 | Cₖₛ Uₖₛ T_{ds} A_{ds} G_{ds} C_{ds} | 1.25 | 65 | 114 | 105 | 78 | 51 | 63 | 13 |
| | A_{ds} C_{ds} T_{ds} G_{ds} G_{ds} C_{ds} | 3.75 | 11 | 107 | 103 | 81 | 137 | 71 | |
| | Cₖₛ U_{HA-C16} | 12.5 | 17 | 124 | 68 | 74 | 31 | 64 | |

See legend for Tables 1 and 4 for subscripts and superscript key. “HA-Chol” is a 2′-modification shown below:

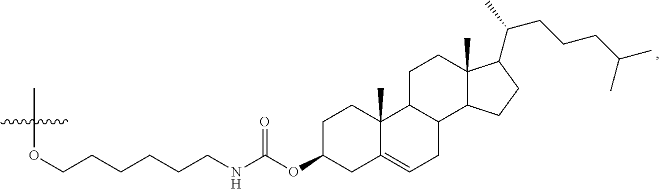 [SMILES: *OCCCCCCNC(=O)O[C@H]1CC[C@@]2(C)C(=CCC3C4CCC([C@H](C)CCCC(C)C)[C@@]4(C)CCC32)C1]

“HA-C10” and “HA-C16” are 2′-modifications shown below:

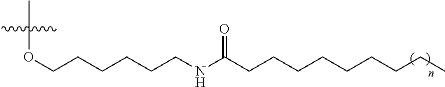

wherein n is 1 in subscript “HA-C10”, and n is 7 in subscript “HA-C16”.

### Example 13: Effects of Oligomeric Compounds In Vivo

The oligomeric compounds described in the table below are complementary to both human and mouse MALAT-1 transcripts. Their effects on MALAT-1 expression were tested in vivo. Wild type male C57bl/6 mice each received a subcutaneous injection of an oligomeric compound at a dose listed in the table below or saline vehicle alone on days 0, 4, and 10 of the treatment period. Each treatment group consisted of three mice. Four days after the last injection, the animals were sacrificed. MALAT-1 RNA expression was analyzed in liver, adipose tissue (fat), and heart by RT-qPCR and normalized to total RNA using RiboGreen (Thermo Fisher Scientific, Carlsbad, Calif.). The average results for each group are shown below as the percent normalized MALAT-1 RNA levels relative to average results for the vehicle treated animals

TABLE 14
MALAT-1 expression in vivo

| | | | MALAT-1 RNA level | | | |
|---|---|---|---|---|---|---|
| | | Dosage | (% Vehicle) | | | SEQ ID |
| Isis No. | Sequence (5′ to 3′) | (μmol/kg) | Liver | Fat | Heart | NO. |
| 556089 | Gₖₛ^{m}CₖₛAₖₛT_{ds}T_{ds}^{m}C_{ds}T_{ds}A_{ds}A_{ds}T_{ds}A_{ds} | 0.4 | 43 | 58 | 83 | 3 |
| | G_{ds}^{m}C_{ds}AₖₛGₖₛ^{m}Cₖ | 1.2 | 22 | 46 | 81 | |
| | | 3.6 | 11 | 31 | 57 | |
| | | 10.8 | 5 | 19 | 27 | |
| 812134 | C16-HA-T_{do} ^{m}C_{do}A_{do}Gₖₛ^{m}CₖₛAₖₛT_{ds} | 0.4 | 40 | 101 | 88 | 4 |
| | T_{ds}^{m}C_{ds}T_{ds}A_{ds}A_{ds}T_{ds}A_{ds}G_{ds}^{m}C_{ds}Aₖₛ | 1.2 | 11 | 51 | 69 | |
| | Gₖₛ^{m}Cₖ | 3.6 | 4 | 16 | 17 | |
| 859299 | C16-HA-Gₖₛ^{m}CₖₛAₖₛT_{ds}T_{ds}^{m}C_{ds}T_{ds}A_{ds} | 0.4 | 43 | 74 | 80 | 3 |
| | A_{ds}T_{ds}A_{ds}G_{ds}^{m}C_{ds}AₖₛGₖₛ^{m}Cₖ | 1.2 | 13 | 44 | 42 | |
| | | 3.6 | 5 | 17 | 14 | |
| 861242 | C16-2x-C6-Gₖₛ^{m}CₖₛAₖₛT_{ds}T_{ds}^{m}C_{ds}T_{ds} | 0.4 | 41 | 73 | 78 | 3 |
| | A_{ds}A_{ds}T_{ds}A_{ds}G_{ds}^{m}C_{ds}AₖₛGₖₛ^{m}Cₖ | 1.2 | 12 | 40 | 45 | |
| | | 3.6 | 5 | 14 | 13 | |
| 861244 | C16-C6-Gₖₛ^{m}CₖₛAₖₛT_{ds}T_{ds}^{m}C_{ds}T_{ds}A_{ds} | 0.4 | 52 | 81 | 76 | 3 |
| | A_{ds}T_{ds}A_{ds}G_{ds}^{m}C_{ds}AₖₛGₖₛ^{m}Cₖ | 1.2 | 13 | 52 | 67 | |
| | | 3.6 | 7 | 18 | 18 | |
| 863406 | C16-2x-C3-Gₖₛ^{m}CₖₛAₖₛT_{ds}T_{ds}^{m}C_{ds}T_{ds} | 0.4 | 41 | 69 | 97 | 3 |
| | A_{ds}A_{ds}T_{ds}A_{ds}G_{ds}^{m}C_{ds}AₖₛGₖₛ^{m}Cₖ | 1.2 | 15 | 54 | 63 | |
| | | 3.6 | 6 | 21 | 26 | |
| 863407 | C16-C3-Ab-Gₖₛ^{m}CₖₛAₖₛT_{ds}T_{ds}^{m}C_{ds}T_{ds} | 0.4 | 49 | 70 | 109 | 3 |
| | A_{ds}A_{ds}T_{ds}A_{ds}G_{ds}^{m}C_{ds}AₖₛGₖₛ^{m}Cₖ | 1.2 | 18 | 41 | 67 | |
| | | 3.6 | 6 | 28 | 32 | |

See legend for Tables 1 and 4 for subscripts and superscript key. The structures of the conjugate linkers and conjugate moieties are shown below.
The structure of “C16-HA-” is:

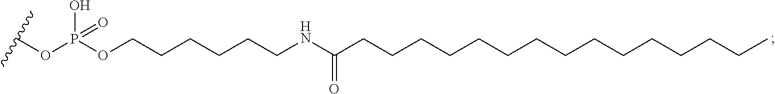 [SMILES: *OP(=O)(O)OCCCCCCNC(=O)CCCCCCCCCCCCCCC]

the structures of “C16-2x-C6-” and “C16-2x-C3-” are:

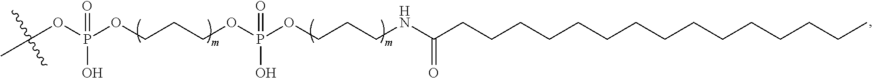

wherein m=2 in “C16-2x-C6-”; and m=1 in “C16-2x-C3-”;
the structure of “C16-C6-” is:

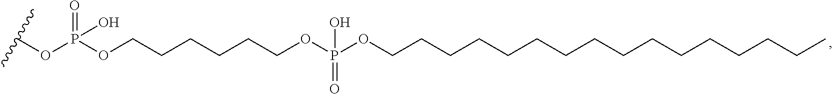 [SMILES: *OP(=O)(O)OCCCCCCOP(=O)(O)OCCCCCCCCCCCCCCCC]

and the structure of “C16-C3-Ab” is:

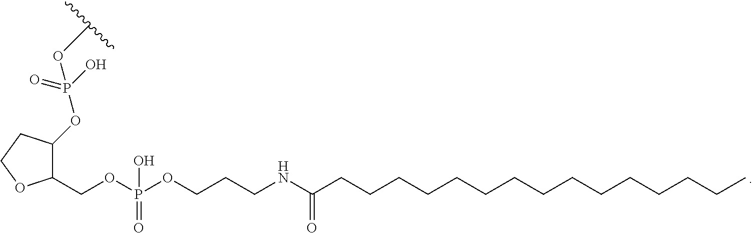 [SMILES: *OP(=O)(O)OC1CCOC1COP(=O)(O)OCCCNC(=O)CCCCCCCCCCCCCCC]

TABLE 15
MALAT-1 ED_{50's}/fold change

| | 50% Target Reduction | | | | | | |
|---|---|---|---|---|---|---|---|
| | Heart ED₅₀ | Fat ED₅₀ | Liver ED₅₀ | Heart fold | Fat fold | Liver fold | |
| Isis No. | (mg/kg) | (mg/kg) | (mg/kg) | change | change | change | Seq ID No. |
| 556089 | 23.3 | 4.3 | 1.53 | | | | 3 |
| 812134 | 6.7 | 6.4 | 2.18 | 3.5 | 0.7 | 0.7 | 4 |
| 859299 | 5.8 | 5.7 | 1.89 | 4.0 | 0.7 | 0.8 | 3 |
| 861242 | 6.1 | 5.3 | 1.86 | 3.8 | 0.8 | 0.8 | 3 |
| 861244 | 8.9 | 7.3 | 2.46 | 2.6 | 0.6 | 0.6 | 3 |
| 863406 | 10.6 | 6.6 | 1.76 | 2.2 | 0.6 | 0.9 | 3 |
| 863407 | 12.8 | 5.8 | 2.31 | 1.8 | 0.7 | 0.7 | 3 |

TABLE 16
MALAT-1 ED_{10's} / fold change

| | 90% Target Reduction | | | | | | |
|---|---|---|---|---|---|---|---|
| | Heart ED₁₀ | Fat ED₁₀ | Liver ED₁₀ | Heart fold | Fat fold | Liver fold | |
| Isis No. | (mg/kg) | (mg/kg) | (mg/kg) | change | change | change | Seq ID No. |
| 556089 | 247 | 281 | 20 | | | | 3 |
| 812134 | 29 | 88 | 10 | 8.6 | 3.2 | 2.0 | 4 |
| 859299 | 25 | 35 | 9 | 9.7 | 8.1 | 2.2 | 3 |
| 861242 | 29 | 30 | 9 | 8.6 | 9.5 | 2.2 | 3 |
| 861244 | 47 | 37 | 10 | 5.2 | 7.6 | 2.1 | 3 |
| 863406 | 41 | 68 | 11 | 6.0 | 4.1 | 1.8 | 3 |
| 863407 | 45 | 80 | 12 | 5.5 | 3.5 | 1.6 | 3 |

### Example 14: Effects of Oligomeric Compounds Comprising Various Conjugate Groups In Vivo

The oligomeric compounds described in the table below are complementary to both human and mouse Dystrophia Myotonica-Protein Kinase (DMPK) transcript. Their effects on DMPK expression were tested in vivo. Wild type Balb/c mice each received either an intravenous (IV) or a subcutaneous (SC) injection of 10 mg/kg of oligomeric compound or saline vehicle alone. Each animal received one dose per week for 3½ weeks, for a total of 4 doses. Each treatment group consisted of three or four mice. Two days after the last dose, the animals were sacrificed. DMPK mRNA expression was analyzed in the heart and quadriceps by RT-qPCR and normalized to total RNA using RiboGreen (Thermo Fisher Scientific, Carlsbad, Calif.). The average results for each group are shown below as the percent normalized DMPK RNA levels relative to average results for the vehicle treated animals. The data below show that the oligomeric compounds comprising certain conjugate groups were more potent when administered through IV or SC routes of administration compared to a parent oligomeric compound that does not comprise a conjugate group.

TABLE 17
Conjugated Oligomeric Compounds Targeted to DMPK

| Compound | | | SEQ ID |
|---|---|---|---|
| No. | Sequence (5′ to 3′) | Conjugate | NO. |
| 486178 | Aₖₛ^{m}CₖₛAₖₛA_{ds}T_{ds}A_{ds}A_{ds}A_{ds}T_{ds}A_{ds}^{m}C_{ds}^{m}C_{ds}G_{ds}AₖₛGₖₛGₖ | None | 11 |
| 819733 | Chol-TEG-T_{do}^{m}C_{do}A_{do}Aₖₛ^{m}CₖₛAₖₛA_{ds}T_{ds}A_{ds}A_{ds}A_{ds}T_{ds}A_{ds}^{m}C_{ds} | Chol-TEG | 12 |
| | ^{m}C_{ds}G_{ds}AₖₛGₖₛGₖ | | |
| 819734 | Toco-TEG-T_{do}^{m}C_{do}A_{do}Aₖₛ^{m}CₖₛAₖₛA_{ds}T_{ds}A_{ds}A_{ds}A_{ds}T_{ds}A_{ds}^{m}C_{ds} | Toco-TEG | 12 |
| | ^{m}C_{ds}G_{ds}AₖₛGₖₛGₖ | | |
| 853212 | C16-TEG-T_{do}^{m}C_{do}A_{do}Aₖₛ^{m}CₖₛAₖₛA_{ds}T_{ds}A_{ds}A_{ds}A_{ds}T_{ds}A_{ds}^{m}C_{ds}^{m}C_{ds} | C16-TEG | 12 |
| | G_{ds}AₖₛGₖₛGₖ | | |
| 853213 | C16-HA-T_{do}^{m}C_{do}A_{do}Aₖₛ^{m}CₖₛAₖₛA_{ds}T_{ds}A_{ds}A_{ds}A_{ds}T_{ds}A_{ds}^{m}C_{ds}^{m}C_{ds} | C16-HA | 12 |
| | G_{ds}AₖₛGₖₛGₖ | | |

See legend for Tables 1 and 4 for subscripts and superscript key. The structures of the conjugate linkers and conjugate moieties are shown in examples 1-3, and 13 above.

TABLE 18
DMPK expression in vivo

| | |
|---|---|
| Dose | Avg % Ctrl in Various Tissues |

| Group | mpk/wk | Route | Heart | Quad |
|---|---|---|---|---|
| Saline | | IV | 100.0 | 100.0 |
| 486178 | 10 | IV | 78.0 | 48.7 |
| 819733 | 10 | IV | 25.9 | 8.2 |
| 819734 | 10 | IV | 38.7 | 18.4 |
| 853212 | 10 | IV | 45.8 | 15.0 |
| 853213 | 10 | IV | 33.1 | 11.3 |
| Saline | | SC | 100.0 | 100.0 |
| 486178 | 10 | SC | 74.1 | 51.8 |
| 819733 | 10 | SC | 81.2 | 67.0 |
| 819734 | 10 | SC | 84.8 | 76.0 |
| 853212 | 10 | SC | 54.8 | 29.6 |
| 853213 | 10 | SC | 43.9 | 26.2 |

### Example 15: Effects of Oligomeric Compounds Comprising a Lipophilic Conjugate Group In Vivo

The oligomeric compounds described in the table below are complementary to both human and mouse Dystrophia Myotonica-Protein Kinase (DMPK) transcript. Their effects on DMPK expression were tested in vivo. Wild type Balb/c mice each received a subcutaneous injection of an oligomeric compound at a dosage listed in the table below or saline vehicle alone. Each animal received one dose per week for 3½ weeks, for a total of 4 doses. Each treatment group consisted of three or four mice. Two days after the last dose, the animals were sacrificed. DMPK mRNA expression was analyzed in the heart, quadriceps (quad), diaphragm, tibialis (tibia), and gastrocnemius (gastroc) by RT-qPCR and normalized to total RNA using RiboGreen (Thermo Fisher Scientific, Carlsbad, Calif.). The average results for each group are shown below as the percent normalized DMPK RNA levels relative to average results for the vehicle treated animals. The average results for each group at each dose were then used to calculate the ED50 or ED30. The data below show that the oligomeric compounds comprising a lipophilic conjugate group were more potent in various tissues compared to a parent oligomeric compound that does not comprise a lipophilic conjugate group.

TABLE 19
Conjugated Oligomeric Compounds Targeted to DMPK

| Compound | | | SEQ ID |
|---|---|---|---|
| No. | Sequence (5′ to 3′) | Conjugate | NO. |
| 486178 | Aₖₛ^{m}CₖₛAₖₛA_{ds}T_{ds}A_{ds}A_{ds}A_{ds}T_{ds}A_{ds}^{m}C_{ds}^{m}C_{ds}G_{ds}AₖₛGₖₛGₖ | None | 11 |
| 853213 | C16-HA-T_{do}^{m}C_{do}A_{do}Aₖₛ^{m}CₖₛAₖₛA_{ds}T_{ds}A_{ds}A_{ds}A_{ds}T_{ds}A_{ds}^{m}C_{ds} | C16-HA | 12 |
| | ^{m}C_{ds}G_{ds}AₖₛGₖₛGₖ | | |
| 877864 | C16-HA-Aₖₛ^{m}CₖₛAₖₛA_{ds}T_{ds}A_{ds}A_{ds}A_{ds}T_{ds}A_{ds}^{m}C_{ds}^{m}C_{ds}G_{ds} | C16-HA | 11 |
| | AₖₛGₖₛGₖ | | |
| 877865 | C16-2X-C6-Aₖₛ^{m}CₖₛAₖₛA_{ds}T_{ds}A_{ds}A_{ds}A_{ds}T_{ds}A_{ds}^{m}C_{ds}^{m}C_{ds}G_{ds}Aₖₛ | C16-2x-C6 | 11 |
| | GₖₛGₖ | | |

See legend for Tables 1 and 4 for subscripts and superscript key. The structures of the conjugate linkers and conjugate moieties are shown in examples 1-3, and 13 above.

TABLE 20
DMPK expression in vivo

| | Dose | Avg % Ctrl in Various Tissues | | | | |
|---|---|---|---|---|---|---|
| Group | mpk/wk | Heart | Quad | Gastroc | Tibia | Diaphragm |
| Saline | | 100.0 | 100.0 | 100.0 | 100.0 | 100.0 |
| 486178 | 10 | 65.4 | 54.6 | 46.9 | 62.9 | 59.4 |
| | 20 | 42.0 | 28.9 | 57.9 | 39.9 | 36.5 |
| | 40 | 45.4 | 13.4 | 13.9 | 18.8 | 19.8 |
| 853213 | 5 | 74.6 | 69.6 | 96.7 | 76.0 | 48.8 |
| | 10 | 46.7 | 21.7 | 23.9 | 31.0 | 30.1 |
| | 15 | 40.8 | 7.9 | 11.6 | 16.8 | 19.1 |
| 877864 | 5 | 53.5 | 47.6 | 45.2 | 55.1 | 45.5 |
| | 10 | 25.9 | 14.2 | 11.8 | 23.4 | 18.0 |
| | 20 | 14.5 | 4.8 | 2.2 | 6.6 | 8.5 |
| 877865 | 5 | 54.9 | 59.8 | 65.4 | 67.1 | 41.5 |
| | 10 | 30.3 | 22.5 | 17.3 | 29.9 | 25.6 |
| | 20 | 19.3 | 7.5 | 2.2 | 6.6 | 7.6 |

“mpk/wk” designates milligrams per kilogram of bodyweight per week.

TABLE 21
DMPK expression in vivo

| | ED50 or ED30 in Various Tissues (mpk/wk) | | | | |
|---|---|---|---|---|---|
| | Heart | Quad | Gastroc | Tibia | Diaphragm |
| Group | (ED50) | (ED50) | (ED50) | (ED50) | (ED30) |
| 486178 | 15.8 | 11.0 | 12.1 | 14.9 | 27.0 |
| 853213 | 10.3 | 6.8 | 8.4 | 7.8 | 10.0 |
| 877864 | 5.1 | 4.1 | 3.8 | 5.4 | 8.0 |
| 877865 | 5.5 | 5.9 | 6.2 | 7.0 | 8.1 |

### Example 16: Effects of Oligomeric Compounds Comprising a Lipophilic Conjugate Group In Vivo

Oligomeric compounds 486178 and 877864, described in the table below, are complementary to both human and mouse Dystrophia Myotonica-Protein Kinase (DMPK) transcript. Oligomeric compound 549144 is a scrambled control oligomeric compound. The effects of compounds 549144, 486178, and 877864 on DMPK expression were tested in vivo. Wild type Sprague-Dawley rats each received a subcutaneous injection of an oligomeric compound at a dosage listed in the table below or a PBS vehicle alone. Each animal received one dose per week for 3½ weeks, for a total of 4 doses. Each treatment group consisted of four rats. Two days after the last dose, the animals were sacrificed. DMPK mRNA expression was analyzed in the liver, quadriceps, and heart by RT-qPCR and normalized to total RNA using RiboGreen (Thermo Fisher Scientific, Carlsbad, Calif.). The average results for each group are shown below as the percent normalized DMPK RNA levels relative to average results for the vehicle treated animals. The data below show that the oligomeric compounds comprising a lipophilic conjugate group were more potent in the liver, heart, and quadriceps (quad) compared to the parent oligomeric compound that does not comprise a lipophilic conjugate group.

TABLE 22
Conjugated Oligomeric Compounds Targeted to DMPK

| Compound | | | SEQ ID |
|---|---|---|---|
| No. | Sequence (5′ to 3′) | Conjugate | NO. |
| 486178 | Aₖₛ^{m}CₖₛAₖₛA_{ds}T_{ds}A_{ds}A_{ds}A_{ds}T_{ds}A_{ds}^{m}C_{ds}^{m}C_{ds}G_{ds}AₖₛGₖₛGₖ | None | 11 |
| 549144 | GₖₛGₖₛ^{m}Cₖₛ^{m}C_{ds}A_{ds}A_{ds}T_{ds}A_{ds}^{m}C_{ds}G_{ds}^{m}C_{ds}^{m}C_{ds}G_{ds}Tₖₛ^{m}CₖₛAₖ | None | 40 |
| 877864 | C16-HA-Aₖₛ^{m}CₖₛAₖₛA_{ds}T_{ds}A_{ds}A_{ds}A_{ds}T_{ds}A_{ds}^{m}C_{ds}^{m}C_{ds}G_{ds} | C16-HA | 11 |
| | AₖₛGₖₛGₖ | | |

See legend for Tables 1 and 4 for subscripts and superscript key. The structures of the conjugate linkers and conjugate moieties are shown in examples 1-3 above.

TABLE 23
DMPK expression in vivo

| | |
|---|---|
| Dose | Avg % Ctrl in Various Tissues |

| Group | mpk/wk | Liver | Heart | Quad |
|---|---|---|---|---|
| PBS | | 100.0 | 100.0 | 100.0 |
| 549144 | 60 | 104 | 97 | 119 |
| 486178 | 10 | 69 | 72 | 74 |
| | 30 | 36 | 45 | 49 |
| | 60 | 17 | 28 | 27 |
| 877864 | 3 | 104 | 91 | 78 |
| | 10 | 68 | 58 | 46 |
| | 30 | 24 | 23 | 17 |

“mpk/wk” designates milligrams per kilogram of bodyweight per week.

### Example 17: Effects of Oligomeric Compounds Comprising Lipophilic Conjugate Groups of Various Sizes In Vivo

The oligomeric compounds described in the table below are complementary to both human and mouse Malat-1 transcript. Their effects on Malat-1 expression were tested in vivo. Male C57BL/6 mice each received a subcutaneous injection of an oligomeric compound at a dosage listed in the table below or saline vehicle alone. Each animal received one dose per week for 3 weeks, for a total of 3 doses. Each treatment group consisted of three mice. Two days after the last dose, the animals were sacrificed. Malat-1 mRNA expression was analyzed in the heart, adipose, and quadriceps (quad), by RT-qPCR and normalized to total RNA using Cyclophilin. The average results for each group are shown below as the percent normalized Malat-1 RNA levels relative to average results for the vehicle treated animals. The data below show that lipophilic conjugate groups of various lengths improve activity across multiple tissues.

TABLE 24
Conjugated Oligomeric Compounds Targeted to Malat-1

| Compound | | | SEQ ID |
|---|---|---|---|
| No. | Sequence (5′ to 3′) | Conjugate | NO. |
| 556089 | Gₖₛ^{m}CₖₛAₖₛT_{ds}T_{ds}^{m}C_{ds}T_{ds}A_{ds}A_{ds}T_{ds}A_{ds}G_{ds}^{m}C_{ds}AₖₛGₖₛ^{m}Cₖ | None | 3 |
| 929856 | C8-HA- Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | C8-HA | 3 |
| | AₖₛGₖₛ^{m}Cₖ | | |
| 929847 | C10-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | C10-HA | 3 |
| | AₖₛGₖₛ^{m}Cₖ | | |
| 929853 | C12-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | C12-HA | 3 |
| | AₖₛGₖₛ^{m}Cₖ | | |
| 929854 | C14-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | C14-HA | 3 |
| | AₖₛGₖₛ^{m}Cₖ | | |
| 859299 | C16-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | C16-HA | 3 |
| | AₖₛGₖₛ^{m}Cₖ | | |
| 929855 | C18-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | C18-HA | 3 |
| | AₖₛGₖₛ^{m}Cₖ | | |
| 929857 | C20-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | C20-HA | 3 |
| | AₖₛGₖₛ^{m}Cₖ | | |
| 929858 | C22-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | C22-HA | 3 |
| | AₖₛGₖₛ^{m}Cₖ | | |
| 929840 | C22-HA-T_{do}^{m}C_{do}A_{do}Gₖₛ^{m}CₖₛAₖₛT_{ds}T_{ds}^{m}C_{ds}T_{ds}A_{ds}A_{ds}T_{ds}A_{ds} | C22-HA | 4 |
| | G_{ds}^{m}C_{ds}AₖₛGₖₛ^{m}Cₖ | | |

See legend for Tables 1 and 4 for subscripts and superscript key.
The structures of the conjugates are shown below:

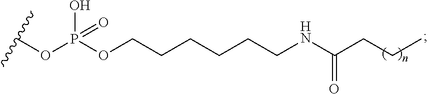

wherein n=5 for C8-HA, n=7 for C10-HA, n=9 for C12-HA, n=11 for C14-HA, n=13 for C16-HA, n=15 for C18-HA, n=17 for C20-HA, and n=19 for C22-HA.

TABLE 25

| | |
|---|---|
| Dose | Avg % Ctrl in Various Tissues |

| Group | μmol/kg/wk | Heart | Adipose | Quad |
|---|---|---|---|---|
| Saline | | 100.0 | 100.0 | 100.0 |
| 556089 | 3.6 | 34 | 15 | 38 |
| 929856 | 1.2 | 78 | 33 | 57 |
| | 3.6 | 45 | 14 | 31 |
| 929847 | 1.2 | 85 | 31 | 64 |
| | 3.6 | 37 | 13 | 34 |
| 929853 | 1.2 | 57 | 24 | 62 |
| | 3.6 | 24 | 12 | 14 |
| 929854 | 1.2 | 58 | 46 | 59 |
| | 3.6 | 25 | 16 | 21 |
| 859299 | 1.2 | 55 | 20 | 42 |
| | 3.6 | 21 | 11 | 10 |
| 929855 | 1.2 | 40 | 17 | 34 |
| | 3.6 | 15 | 9 | 12 |
| 929857 | 1.2 | 40 | 26 | 53 |
| | 3.6 | 16 | 15 | 8 |
| 929858 | 1.2 | 49 | 23 | 76 |
| | 3.6 | 16 | 14 | 17 |
| 929840 | 1.2 | 49 | 30 | 75 |
| | 3.6 | 17 | 14 | 19 |

### Example 18: Effects of Oligomeric Compounds Comprising Lipophilic Conjugate Groups of Various Sizes and Saturation In Vivo

The oligomeric compounds described in the table below are complementary to both human and mouse Malat-1 transcript. Their effects on Malat-1 expression were tested in vivo. Male C57BL/6 mice each received a subcutaneous injection of an oligomeric compound at a dosage listed in the table below or saline vehicle alone. Each animal received one dose per week for 3 weeks, for a total of 3 doses. Each treatment group consisted of three mice. Two days after the last dose, the animals were sacrificed. Malat-1 mRNA expression was analyzed in the heart, white adipose (WA), quadriceps (quad), and liver by RT-qPCR and normalized to total RNA using RiboGreen (Thermo Fisher Scientific, Carlsbad, Calif.). The average results for each group are shown below as the percent normalized Malat-1 RNA levels relative to average results for the vehicle treated animals. The data below show that lipophilic conjugate groups of various lengths and unsaturation improve activity compared to an unconjugated parent compound.

TABLE 26
Conjugated Oligomeric Compounds Targeted to Malat-1

| Compound | | | SEQ |
|---|---|---|---|
| No. | Sequence (5′ to 3′) | Conjugate | ID NO. |
| 556089 | Gₖₛ^{m}CₖₛAₖₛT_{ds}T_{ds}^{m}C_{ds}T_{ds}A_{ds}A_{ds}T_{ds}A_{ds}G_{ds}^{m}C_{ds}AₖₛGₖₛ^{m}Cₖ | None | 3 |
| 859299 | C16-HA-Gₖₛ^{m}CₖₛAₖₛT_{ds}T_{ds}^{m}C_{ds}T_{ds}A_{ds}A_{ds}T_{ds}A_{ds} | C16-HA | 3 |
| | G_{ds}^{m}C_{ds}AₖₛGₖₛ^{m}Cₖ | | |
| 950439 | Palmitoleoyl-HA-Gₖₛ^{m}CₖₛAₖₛT_{ds}T_{ds}^{m}C_{ds}T_{ds}A_{ds}A_{ds}T_{ds}A_{ds} | C16, ω 7 | 3 |
| | G_{ds}^{m}C_{ds}AₖₛGₖₛ^{m}Cₖ | (Palmitoleoyl- | |
| | | HA-) | |
| 950437 | Linoleoyl-HA-Gₖₛ^{m}CₖₛAₖₛT_{ds}T_{ds}^{m}C_{ds}T_{ds}A_{ds}A_{ds}T_{ds}A_{ds} | C18, ω 6, 9 | 3 |
| | G_{ds}^{m}C_{ds}AₖₛGₖₛ^{m}Cₖ | (Linoleoyl- | |
| | | HA-) | |
| 950641 | Linolenyl-HA-Gₖₛ^{m}CₖₛAₖₛT_{ds}T_{ds}^{m}C_{ds}T_{ds}A_{ds}A_{ds}T_{ds}A_{ds} | C18, ω6, 9 | 3 |
| | G_{ds}^{m}C_{ds}AₖₛGₖₛ^{m}Cₖ | (Linolenyl- | |
| | | HA-) | |
| 950438 | Arachidonyl-HA-Gₖₛ^{m}CₖₛAₖₛT_{ds}T_{ds}^{m}C_{ds}T_{ds}A_{ds}A_{ds}T_{ds}A_{ds}G_{ds} | C20, ω 6, 9, | 3 |
| | ^{m}C_{ds}AₖₛGₖₛ^{m}Cₖ | 12, 15 | |
| | | (Arachidonyl- | |
| | | HA-) | |
| 867593 | DHA6 ω -HA- | C22, ω 3, 6, 9, | 2 |
| | T_{do}^{m}C_{do}A_{do}Gₖₛ^{m}CₖₛAₖₛT_{ds}T_{ds}^{m}C_{ds}T_{ds}A_{ds}A_{ds}T_{ds} | 12, 15, 18 | |
| | A_{ds}G_{ds}^{m}C_{ds}AₖₛGₖₛ^{m}Cₖ | (DHA 6ω - | |
| | | HA-) | |

See legend for Tables 1 and 4 for subscripts and superscript key.
The structures of the conjugates are shown below:
“Palmitoleoyl-HA-” has the following structure:

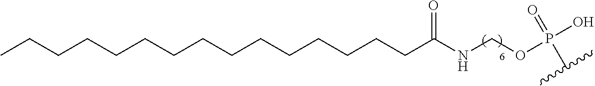

“Linoleoyl-HA-” has the following structure:

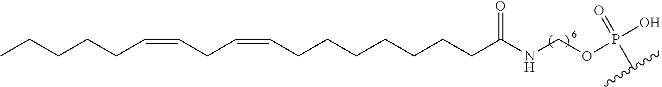

“Linolenyl-HA-” has the following structure:

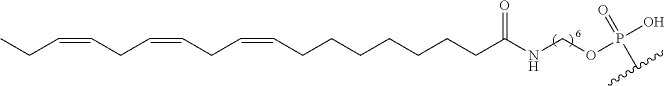

“Arachidonyl-HA-” has the following structure:

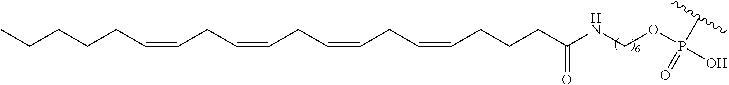

“DHA 6ω-HA-” has the following structure:

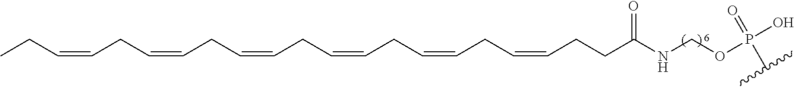

TABLE 27
Conjugated Oligomeric Compounds Targeted to Malat-1

| | |
|---|---|
| Dose | Avg % Ctrl in Various Tissues |

| Group | μmol/kg/wk | Heart | WA | Quad | Liver |
|---|---|---|---|---|---|
| Saline | | 100.0 | 100.0 | 100.0 | 100.0 |
| 556089 | 3.6 | 29.2 | 13.0 | 13.0 | 13.0 |
| 859299 | 1.2 | 43.5 | 18.8 | 18.8 | 18.8 |
| | 3.6 | 11.9 | 8.7 | 8.7 | 8.7 |
| 950439 | 1.2 | 41.2 | 30.3 | 27.3 | 27.3 |
| | 3.6 | 20.0 | 14.8 | 10.6 | 10.6 |
| 950437 | 1.2 | 48.6 | 27.3 | 29.9 | 29.9 |
| | 3.6 | 19.7 | 10.6 | 11.4 | 11.4 |
| 950641 | 1.2 | 41.9 | 29.9 | 27.9 | 27.9 |
| | 3.6 | 21.9 | 11.4 | 14.1 | 14.1 |
| 950438 | 1.2 | 48.0 | 27.9 | 29.6 | 29.6 |
| | 3.6 | 15.7 | 14.1 | 11.4 | 11.4 |
| 867593 | 1.2 | 51.7 | 29.6 | 27.9 | 27.9 |
| | 3.6 | 24.5 | 11.4 | 11.1 | 11.1 |

### Example 19: Effects of Oligomeric Compounds Comprising Lipophilic Conjugate Groups of Various Amounts of Unsaturation In Vivo

The oligomeric compounds described in the table below are complementary to both human and mouse Malat-1 transcript. Their effects on Malat-1 expression were tested in vivo. Male C57BL/6 mice each received a subcutaneous injection of an oligomeric compound at a dosage listed in the table below or saline vehicle alone. Each animal received one dose per week for 3 weeks, for a total of 3 doses. Each treatment group consisted of three mice. Two days after the last dose, the animals were sacrificed. Malat-1 mRNA expression was analyzed in the heart and quadriceps by RT-qPCR and normalized to total RNA using RiboGreen (Thermo Fisher Scientific, Carlsbad, Calif.). The average results for each group are shown below as the percent normalized Malat-1 RNA levels relative to average results for the vehicle treated animals. The data below show that lipophilic conjugate groups having trans-unsaturated bonds improve activity compared to an unconjugated parent.

TABLE 28
Conjugated Oligomeric Compounds Targeted to Malat-1

| Compound | | | SEQ ID |
|---|---|---|---|
| No. | Sequence (5′ to 3′) | Conjugate | NO. |
| 556089 | Gₖₛ^{m}CₖₛAₖₛT_{ds}T_{ds}^{m}C_{ds}T_{ds}A_{ds}A_{ds}T_{ds}A_{ds}G_{ds}^{m}C_{ds}AₖₛGₖₛ^{m}Cₖ | None | 3 |
| 859299 | Gₖₛ^{m}CₖₛAₖₛT_{ds}T_{ds}^{m}C_{ds}T_{ds}A_{ds}A_{ds}T_{ds}A_{ds}G_{ds}^{m}C_{ds}AₖₛGₖₛ^{m}Cₖ | C16-HA | 3 |
| 950642 | Elaidoyl-HA-Gₖₛ^{m}CₖₛAₖₛT_{ds}T_{ds}^{m}C_{ds}T_{ds}A_{ds}A_{ds}T_{ds}A_{ds} | C18, ω 9 | 3 |
| | G_{ds}^{m}C_{ds}AₖₛGₖₛ^{m}Cₖ | (Elaidoyl- | |
| | | HA-) | |
| 950643 | Linoelaidoyl-HA-Gₖₛ^{m}CₖₛAₖₛT_{ds}T_{ds}^{m}C_{ds}T_{ds}A_{ds}A_{ds}T_{ds}A_{ds}G_{ds} | C18, ω 6, 9 | 3 |
| | ^{m}C_{ds}AₖₛGₖₛ^{m}Cₖ | (Linoelaidoyl- | |
| | | HA-) | |

See legend for Tables 1 and 4 for subscripts and superscript key.
The structures of the conjugates are shown below:
“Elaidoyl-HA-” has the following structure:

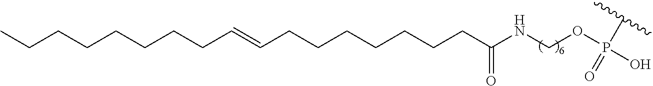

“Linoelaidoyl-HA-” has the following structure:

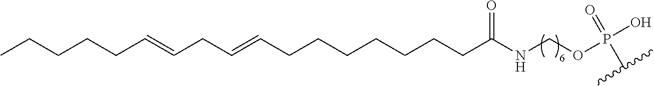

TABLE 29
Conjugated Oligomeric Compounds Targeted to Malat-1

| | |
|---|---|
| Dose | Avg % Ctrl in Various Tissues |

| Group | μmol/kg/wk | Heart | Quad |
|---|---|---|---|
| Saline | | 100.0 | 100.0 |
| 556089 | 3.6 | 37.0 | 33.7 |
| 859299 | 1.2 | 46.1 | 59.1 |
| | 3.6 | 15.9 | 23.1 |
| 950642 | 1.2 | 51.0 | 60.1 |
| | 3.6 | 18.3 | 20.4 |
| 950643 | 1.2 | 55.8 | 79.0 |
| | 3.6 | 25.6 | 26.1 |

### Example 20: Effects of Oligomeric Compounds Comprising Lipophilic Conjugate Groups Having Various Linking Groups In Vivo

The oligomeric compounds described in the table below are complementary to both human and mouse Malat-1 transcript. Their effects on Malat-1 expression were tested in vivo. Male C57BL/6 mice each received a single intravenous injection (IV) of 12.5 mg/kg of an oligomeric compound listed in the table below or saline vehicle alone. 72 hours after receiving the IV dose, the animals were sacrificed. Malat-1 mRNA expression was analyzed in various tissues by RT-qPCR and normalized to total RNA using RiboGreen (Thermo Fisher Scientific, Carlsbad, Calif.). The average results for each group are shown below as the percent normalized Malat-1 RNA levels relative to average results for the vehicle treated animals. The data below show that lipophilic conjugate using different linkers improve activity compared to an unconjugated parent.

TABLE 30
Conjugated Oligomeric Compounds Targeted to Malat-1

| Compound | | | SEQ |
|---|---|---|---|
| No. | Sequence (5′ to 3′) | Conjugate | ID NO. |
| 556089 | Gₖₛ^{m}CₖₛAₖₛT_{ds}T_{ds}^{m}C_{ds}T_{ds}A_{ds}A_{ds}T_{ds}A_{ds}G_{ds}^{m}C_{ds}AₖₛGₖₛ^{m}Cₖ | None | 3 |
| 953626 | Chol-TEG-Gₖₛ^{m}CₖₛAₖₛT_{ds}T_{ds}^{m}C_{ds}T_{ds}A_{ds}A_{ds}T_{ds}A_{ds}G_{ds}^{m}C_{ds}Aₖₛ | Chol-TEG | 3 |
| | Gₖₛ^{m}Cₖ | | |
| 867613 | Chol-TEG-T_{do}^{m}C_{do}A_{do}Gₖₛ^{m}CₖₛAₖₛT_{ds}T_{ds}^{m}C_{ds}T_{ds}AdA_{ds}T_{ds}A_{ds} | Chol-TEG | 4 |
| | G_{ds}^{m}C_{ds}AₖₛGₖₛ^{m}Cₖ | | |
| 861245 | Chol-TEG-C6-Gₖₛ^{m}CₖₛAₖₛT_{ds}T_{ds}^{m}C_{ds}T_{ds}A_{ds}A_{ds}T_{ds}A_{ds}G_{ds} | Chol-TEG- | 3 |
| | ^{m}C_{ds}AₖₛGₖₛ^{m}Cₖ | C6 | |

See legend for Tables 1 and 4 for subscripts and superscript key.
The structure of “Choi-TEG-C6-” is shown below:

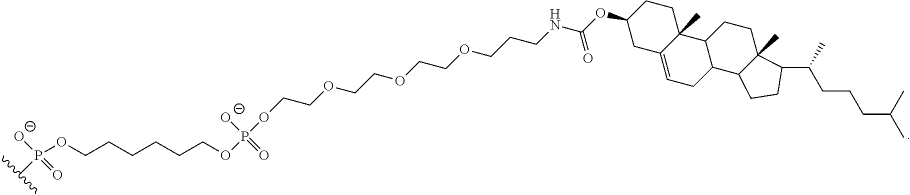 [SMILES: *P(=O)([O-])OCCCCCCOP(=O)([O-])OCCOCCOCCOCCCNC(=O)O[C@H]1CC[C@@]2(C)C(=CCC3C4CCC([C@H](C)CCCC(C)C)[C@@]4(C)CCC32)C1]

The structure of “Chol-TEG-” is shown below:

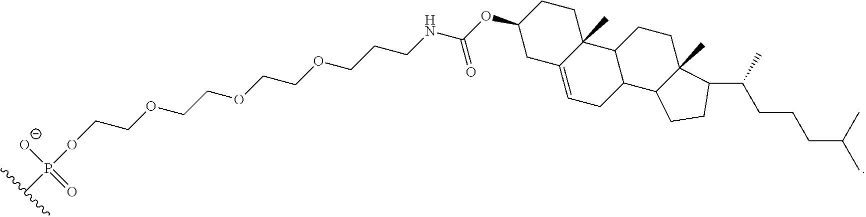 [SMILES: *P(=O)([O-])OCCOCCOCCOCCCNC(=O)O[C@H]1CC[C@@]2(C)C(=CCC3C4CCC([C@H](C)CCCC(C)C)[C@@]4(C)CCC32)C1]

TABLE 31
Conjugated Oligomeric Compounds Targeted to Malat-1

| | |
|---|---|
| Dose | Avg % Ctrl in Various Tissues |

| Group | mg/kg | Heart | Liver | Quad |
|---|---|---|---|---|
| Saline | | 100.0 | 100.0 | 100.0 |
| 556089 | 12.5 | 71.0 | 24.0 | 87.6 |
| 953626 | 12.5 | 69.0 | 10.1 | 46.8 |
| 867613 | 12.5 | 53.8 | 9.0 | 59.6 |
| 861245 | 12.5 | 54.2 | 10.1 | 59.8 |

### Example 21: Effects of Oligomeric Compounds Comprising Lipophilic Conjugate Groups in Heart Tissue In Vivo

The oligomeric compounds described in the table below are complementary to mouse SERCA2 transcript. Their effects on SERCA2 expression were tested in vivo. Six to eight week old C57/B6 mice each received a subcutaneous injection of an oligomeric compound at a dosage listed in the table below or saline vehicle alone. Each animal received a subcutaneous dose on each of days 0, 7, and 14. Each treatment group consisted of four mice. Four days after the last dose, the animals were sacrificed. SERCA2 mRNA expression was analyzed in the heart by RT-qPCR and normalized to total RNA using RiboGreen (Thermo Fisher Scientific, Carlsbad, Calif.). The average results for each group are shown below as the percent normalized SERCA2 RNA levels relative to average results for the vehicle treated animals. The average results for each group at each dose were then used to calculate the ED50. The data below show that the oligomeric compounds comprising a either a saturated or unsaturated lipophilic conjugate group were more potent in the heart compared to the parent oligomeric compound that does not comprise a lipophilic conjugate group.

TABLE 32
Conjugated Oligomeric Compounds Targeted to SERCA2

| Compound | | | SEQ |
|---|---|---|---|
| No. | Sequence (5′to 3′) | Conjugate | ID NO. |
| 854140 | GₖₛGₖₛ^{m}CₖₛA_{ds}A_{ds}T_{ds}T_{ds}G_{ds}G_{ds}T_{ds}G_{ds}T_{ds}T_{ds}TₖₛAₖₛAₖ | None | 41 |
| 946986 | C16-HA-GₖₛGₖₛ^{m}CₖₛA_{ds}A_{ds}T_{ds}T_{ds}G_{ds}G_{ds}T_{ds}G_{ds}T_{ds}T_{ds}TₖₛAₖₛAₖ | C16-HA | 41 |
| 946988 | Ole-HA-GₖₛGₖₛ^{m}CₖₛA_{ds}A_{ds}T_{ds}T_{ds}G_{ds}G_{ds}T_{ds}G_{ds}T_{ds}T_{ds}TₖₛAₖₛAₖ | Ole-HA | 41 |

See legend for Tables 1 and 4 for subscripts and superscript key. See Example 4 for the structure of Ole-HA.

TABLE 33
SERCA2 expression in vivo

| | Dose | Avg % Ctrl in Various Tissues |
|---|---|---|
| Group | μmol/kg | Heart |
| PBS | | 100.0 |
| 854140 | 1.7 | 96 |
| | 5 | 73 |
| | 15 | 30 |
| 946986 | 1.7 | 80 |
| | 5 | 31 |
| | 15 | 8 |
| 946988 | 1.7 | 89 |
| | 5 | 28 |
| | 15 | 9 |

TABLE 34
SERCA2 expression in vivo

| | ED50 in Heart Tissue (mpk/wk) |
|---|---|
| Group | Heart (ED50) |
| 854140 | 50 |
| 946986 | 20 |
| 946988 | 21 |

### Example 22: Effects of Oligomeric Compounds Comprising Lipophilic Conjugate Groups in Various Heart Tissues In Vivo

The oligomeric compounds described in the table below are complementary to both human and mouse Malat-1 transcript. Their effects on Malat-1 expression in the heart were tested in vivo. Male C57BL/6 mice each received two doses over a seven day period of time of 30 mg/kg of an oligomeric compound listed in the table below or saline vehicle alone. 72 hours after receiving the last dose, the animals were sacrificed, and the heart tissue was isolated and collected for analysis. The heart tissue was separated into cardiomyocytes, endothelial cells, fibroblasts, and macrophages. Malat-1 mRNA expression was analyzed in these various cardiac tissues by RT-qPCR and normalized to total RNA using RiboGreen (Thermo Fisher Scientific, Carlsbad, Calif.). The average results for each group are shown below as the percent normalized Malat-1 RNA levels relative to average results for the vehicle treated animals. The data below show that lipophilic conjugate groups improve activity when the lipophilic group is greater than 10 carbons in length.

TABLE 35
Conjugated Oligomeric Compounds Targeted to Malat-1

| Compound | | | SEQ |
|---|---|---|---|
| No. | Sequence (5′ to 3′) | Conjugate | ID NO. |
| 917228 | Gₖₛ^{m}CₖₒAₖₒT_{ds}T_{ds}^{m}C_{ds}T_{ds}A_{ds}A_{ds}T_{ds}A_{ds}G_{ds}^{m}C_{ds}Aₖₒ Gₖₛ^{m}Cₖ | None | 3 |
| 915340 | C16-HA-Gₖₛ^{m}CₖₒAₖₒT_{ds}T_{ds}^{m}C_{ds}T_{ds}A_{ds}A_{ds}T_{ds}A_{ds}G_{ds}^{m}C_{ds}AₖₒGₖₛ^{m}Cₖ | C16-HA | 3 |

See legend for Tables 1 and 4 for subscripts and superscript key.

TABLE 36
Malat-1 expression in various heart tissues in vivo

| | Dose | Avg % Ctrl in Various Heart Cells | |
|---|---|---|---|
| Group | μmol/kg | Heart Cell Type | % Reduction of Malat-1 |
| Saline | | | |
| 917228 | 30 | Cardiomyocyte | 29 |
| | | Endothelial Cells | 73 |
| | | Fibroblasts | 37 |
| | | Macrophages | 77 |
| 915340 | 30 | Cardiomyocyte | 82 |
| | | Endothelial Cells | 91 |
| | | Fibroblasts | 82 |
| | | Macrophages | 92 |

### Example 23: Effects of Oligomeric Compounds Comprising Lipophilic Conjugate Groups Containing Unsaturated Fatty Acids In Vivo

The oligomeric compounds described in the table below are complementary to both human and mouse Malat-1 transcript. Their effects on Malat-1 expression in various tissues were tested in vivo. Male C57BL/6 mice each received a subcutaneous injection of an oligomeric compound at a dosage listed in the table below or saline vehicle alone. Each animal received one dose per week for 3 weeks, for a total of 3 doses. Each treatment group consisted of three mice. Two days after the last dose, the animals were sacrificed. Malat-1 mRNA expression was analyzed in the heart, white adipose tissue (WA), sciatic nerve, quadriceps, and liver by RT-qPCR and normalized to total RNA using RiboGreen (Thermo Fisher Scientific, Carlsbad, Calif.). The average results for each group are shown below as the percent normalized Malat-1 RNA levels relative to average results for the vehicle treated animals. The data below show that lipophilic conjugate groups improve activity when the lipophilic group is greater than 10 carbons in length.

TABLE 36
Conjugated Oligomeric Compounds Targeted to Malat-1

| Compound | | | SEQ |
|---|---|---|---|
| No. | Sequence (5′to 3′) | Conjugate | ID NO. |
| 556089 | Gₖₛ^{m}CₖₛAₖₛT_{ds}T_{ds}^{m}C_{ds}T_{ds}A_{ds}A_{ds}T_{ds}A_{ds}G_{ds}^{m}C_{ds}AₖₛGₖₛ^{m}Cₖ | None | 3 |
| 859299 | C16-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | C16-HA | 3 |
| | AₖₛGₖₛ^{m}Cₖ | | |
| 985288 | Eru-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | Eru-HA- | 3 |
| | AₖₛGₖₛ^{m}Cₖ | | |
| 985289 | Sap-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | Sap-HA- | 3 |
| | AₖₛGₖₛ^{m}Cₖ | | |
| 985286 | Myr-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | Myr-HA- | 3 |
| | AₖₛGₖₛ^{m}Cₖ | | |
| 985287 | (E)-11-octa-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | (E)-11-octa- | 3 |
| | AₖₛGₖₛ^{m}Cₖ | HA- | |
| 968138 | γ-Lin-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | | |
| | AₖₛGₖₛ^{m}Cₖ | γ-Lin-HA- | 3 |
| 985322 | Ner-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | | |
| | AₖₛGₖₛ^{m}Cₖ | Ner-HA- | 3 |

See legend for Tables 1 and 4 for subscripts and superscript key.
The structure of “Eru-HA-” is shown below:

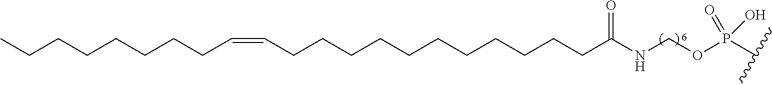

The structure of “Sap-HA-” is shown below:

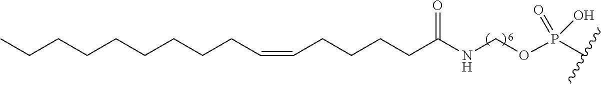

The structure of “Myr-HA-” is shown below:

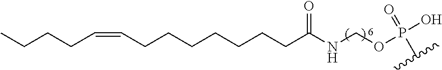

The structure of “(E)-11-octa-HA-” is shown below:

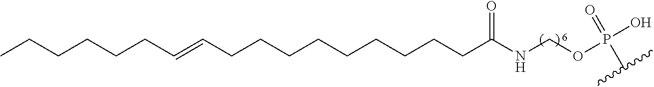

The structure of “γ-Lin-HA-” is shown below:

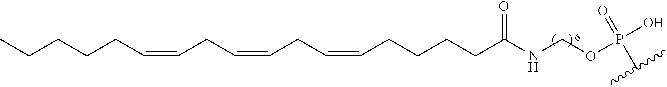

The structure of “Ner-HA-” is shown below:

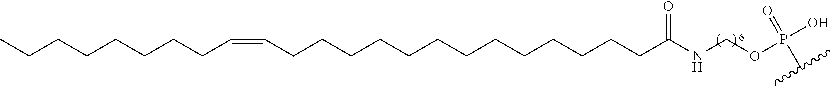

TABLE 37
Conjugated Oligomeric Compounds Targeted to Malat-1

| | |
|---|---|
| Dose | Avg % Ctrl in Various Tissues |

| Group | μmol/kg/wk | Heart | WA | Quad | Liver |
|---|---|---|---|---|---|
| Saline | | 100.0 | 100.0 | 100.0 | 100.0 |
| 556089 | 1.2 | 61.0 | 27.6 | 87.6 | 17.6 |
| | 3.6 | 43.3 | 12.5 | 42.2 | 7.6 |
| 859299 | 1.2 | 52.6 | 30.2 | 59.8 | 10.1 |
| | 3.6 | 21.0 | 8.8 | 19.1 | 4.0 |
| 985288 | 1.2 | 49.4 | 21.7 | 45.4 | 11.6 |
| | 3.6 | 16.9 | 9.5 | 22.8 | 3.6 |
| 985289 | 1.2 | 59.8 | 25.2 | 75.2 | 11.9 |
| | 3.6 | 20.3 | 9.5 | 18.9 | 3.7 |
| 985286 | 1.2 | 57.2 | 39.3 | 73.4 | 9.0 |
| | 3.6 | 21.4 | 10.6 | 31.7 | 4.2 |
| 985287 | 1.2 | 51.7 | 24.8 | 39.6 | 6.5 |
| | 3.6 | 18.1 | 5.7 | 13.6 | 3.3 |
| 968138 | 1.2 | 69.3 | 34.7 | 61.4 | 13.9 |
| | 3.6 | 27.8 | 10.8 | 17.5 | 4.3 |
| 985322 | 1.2 | 68.1 | 29.7 | 51.4 | 13.9 |
| | 3.6 | 24.7 | 13.0 | 28.4 | 4.6 |

### Example 24: Effect of Compounds Comprising a Conjugate Group and a Modified Oligonucleotide Targeting SMN2 in Transgenic Mice Following Systemic Administration

Taiwan type III human transgenic mice were treated by subcutaneous administration with 10-300 mg/kg/week of a modified oligonucleotide listed in the table below or saline (PBS) alone for three weeks and sacrificed 48-72 hours after the last dose. There were 3-4 mice per group. Total RNA from various tissues was extracted and RT-qPCR was performed. The results presented in the table below show that the compounds comprising a C16 conjugate exhibited greater splice modulation activity in various tissues compared to the unconjugated parent.

TABLE 38
Modified oligonucleotides targeting human SMN2

| | | SEQ |
|---|---|---|
| Comp. | | ID |
| No. | Sequence (5′ to 3′) | NO. |
| 387954 | Aₑₛ Tₑₛ Tₑₛ ^{m}Cₑₛ Aₑₛ ^{m}Cₑₛ Tₑₛ Tₑₛ Tₑₛ ^{m}Cₑₛ Aₑₛ Tₑₛ Aₑₛ Aₑₛ Tₑₛ Gₑₛ ^{m}Cₑₛ Tₑₛ Gₑₛ Gₑ | 42 |
| 881068 | C16-HA-Aₑₛ Tₑ ₛTₑ ₛ^{m}Cₑ ₛAₑ ₛ^{m}Cₑ ₛTₑₛ Tₑₛ Tₑₛ ^{m}Cₑₛ Aₑₛ Tₑₛ Aₑₛ Aₑₛ Tₑₛ Gₑₛ ^{m}Cₑₛ Tₑₛ Gₑₛ Gₑ | 42 |
| 881069 | C16-HA -Tₑₛ ^{m}Cₑₛ Aₑₛ ^{m}Cₑₛ Tₑₛ Tₑₛ Tₑₛ ^{m}Cₑₛ Aₑₛ Tₑₛ Aₑₛ Aₑₛ Tₑₛ Gₑₛ ^{m}Cₑₛ Tₑₛ Gₑₛ Gₑ | 43 |
| 881070 | C16-HA -Tₑₛ ^{m}Cₑₛ Aₑₛ ^{m}Cₑₒ Tₑₛ Tₑₒ Tₑₛ ^{m}Cₑₒ Aₑₛ Tₑₒ Aₑₛ Aₑₒ Tₑₛ Gₑₒ ^{m}Cₑₛ Tₑₛ Gₑₛ Gₑ | 43 |

Subscripts in the table above: “s” represents a phosphorothioate internucleoside linkage, “o” represents a phosphate internucleoside linkage, “d” represents a 2′-deoxynucleoside, “e” represents a 2′-MOE modified nucleoside. Superscripts: “m” before a C represents a 5-methylcysteine.

The structure of C16-HA is shown in the examples above:

TABLE 39
Exon 7 inclusion and exclusion

| | | TA Muscle | | | Gastrocnemius | | | Diaphragm | | |
|---|---|---|---|---|---|---|---|---|---|---|
| | | +exon7/ | −exon7/ | | +exon7/ | −exon7/ | | +exon7/ | −exon7/ | |
| Comp. | Dose | total | total | ED₅₀ | total | total | ED₅₀ | total | total | ED₅₀ |
| No. | (mg/kg/wk) | SMN | SMN | (mg/kg) | SMN | SMN | (mg/kg) | SMN | SMN | (mg/kg) |
| PBS | — | 1.0 | 1 | n/a | 1.0 | 1.0 | n/a | 1.0 | 1.0 | n/a |
| 387954 | 30 | 1.0 | 0.9 | 242 | 1.0 | 1.0 | 204 | 1.5 | 0.8 | 122 |
| | 100 | 1.4 | 0.6 | | 1.7 | 0.7 | | 1.9 | 0.6 | |
| | 300 | 2.1 | 0.4 | | 2.3 | 0.3 | | 2.6 | 0.4 | |
| 881068 | 10 | 1.0 | 1.0 | 74 | 0.9 | 1.0 | 69 | 1.1 | 0.9 | 46 |
| | 30 | 1.3 | 0.8 | | 1.3 | 0.8 | | 1.7 | 0.7 | |
| | 100 | 2.2 | 0.2 | | 2.5 | 0.2 | | 2.8 | 0.2 | |
| 881069 | 10 | 1.0 | 1.0 | 56 | 1.0 | 1.0 | 53 | 1.3 | 0.8 | 33 |
| | 30 | 1.4 | 0.7 | | 1.6 | 0.8 | | 2.0 | 0.6 | |
| | 100 | 2.5 | 0.2 | | 2.6 | 0.2 | | 2.9 | 0.1 | |
| 881070 | 10 | 1.1 | 0.9 | 59 | 0.9 | 0.9 | 60 | 1.3 | 1.0 | 26 |
| | 30 | 1.5 | 0.7 | | 1.5 | 0.6 | | 2.3 | 0.6 | |
| | 100 | 2.3 | 0.2 | | 2.6 | 0.2 | | 3.0 | 0.2 | |

### Example 25: Effects of Oligomeric Compounds Comprising Lipophilic Conjugate Groups Containing Steroids In Vivo

The oligomeric compounds described in the table below are complementary to both human and mouse Malat-1 transcript. Their effects on Malat-1 expression in various tissues were tested in vivo. Male C57BL/6 mice each received a subcutaneous injection of an oligomeric compound at a dosage listed in the table below or saline vehicle alone. Each animal received one dose per week for 3 weeks, for a total of 3 doses. Each treatment group consisted of three mice. Two days after the last dose, the animals were sacrificed. Malat-1 mRNA expression was analyzed in the heart, white adipose tissue (WA), sciatic nerve, quadriceps, testicles (testes), and liver by RT-qPCR and normalized to total RNA using RiboGreen (Thermo Fisher Scientific, Carlsbad, Calif.). The average results for each group are shown below as the percent normalized Malat-1 RNA levels relative to average results for the vehicle treated animals

TABLE 36
Conjugated Oligomeric Compounds Targeted to Malat-1

| | | | SEQ |
|---|---|---|---|
| Compound | | | ID |
| No. | Sequence (5′ to 3′) | Conjugate | NO. |
| 556089 | Gₖₛ^{m}CₖₛAₖₛT_{ds}T_{ds}^{m}C_{ds}T_{ds}A_{ds}A_{ds}T_{ds}A_{ds}G_{ds}^{m}C_{ds}AₖₛGₖₛ^{m}Cₖ | None | 3 |
| 859299 | C16-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | C16-HA | 3 |
| | AₖₛGₖₛ^{m}Cₖ | | |
| 968133 | Estradiol-HA- | Estradiol-HA | 3 |
| | Gₖₛ^{m}CₖₛAₖₛT_{ds}T_{ds}^{m}C_{ds}T_{ds}A_{ds}A_{ds}T_{ds}A_{ds}G_{ds}^{m}C_{ds}AₖₛGₖₛ^{m}Cₖ | | |
| 968139 | Dihydrotestosterone-HA-Gₖₛ^{m}CₖₛAₖₛT_{ds}T_{ds}^{m}C_{ds}T_{ds}A_{ds}A_{ds} | Dihydrotestosterone- | 3 |
| | T_{ds}A_{ds}G_{ds}^{m}C_{ds}AₖₛGₖₛ^{m}Cₖ | HA- | |

See legend for Tables 1 and 4 for subscripts and superscript key.
The structure of “Estradiol-HA-” is shown below:

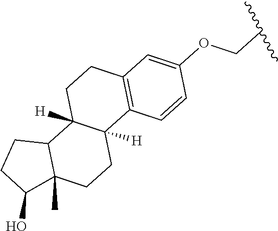 [SMILES: *COc1ccc2c(c1)CC[C@H]1C3CC[C@H](O)[C@@]3(C)CC[C@H]21]

The structure of “Dihydrotestosterone-HA-” is shown below:

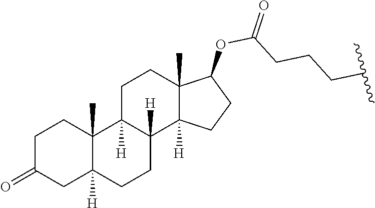

TABLE 37
Conjugated Oligomeric Compounds Targeted to Malat-1 in various tissues

| | Dose | Avg % Ctrl in Various Tissues | | | | |
|---|---|---|---|---|---|---|
| Group | μmol/kg/wk | Heart | WA | Quad | Liver | Testes |
| Saline | | 100.0 | 100.0 | 100.0 | 100.0 | 100.0 |
| 556089 | 1.2 | 61.0 | 27.6 | 87.6 | 17.6 | 57.7 |
| | 3.6 | 43.3 | 12.5 | 42.2 | 7.6 | 33.9 |
| 859299 | 1.2 | 52.6 | 30.2 | 59.8 | 10.1 | 48.7 |
| | 3.6 | 21.0 | 8.8 | 19.1 | 4.0 | 25.0 |
| 968133 | 1.2 | 91.6 | 30.9 | 74.3 | 14.3 | 67.5 |
| | 3.6 | 49.4 | 42.6 | 32.2 | 6.2 | 35.0 |
| 968139 | 1.2 | 80.9 | 33.6 | 80.6 | 11.4 | 61.22 |
| | 3.6 | 47.5 | 15.7 | 45.4 | 4.2 | 27.2 |

### Example 26: Effects of Oligomeric Compounds Comprising Lipophilic Conjugate Groups on Protein Binding

Compound 863776 is a modified oligonucleotide having the sequence and motif described in the table below. Compound 863776 has a C16 lipophilic group conjugated to the 5′ terminus via a hexylamino linker and an Alexa 647 fluorophore conjugated at the 3′-terminus. 10 nM of 863776 was bound to different concentrations of human serum Albumin (HuSA). The top concentration of HuSA used was 1 mM, followed by 3-fold dilutions to establish a 16 point binding curve. The binding curve was then used to determine the HuSA concentration where the ASO and protein were 80-90% bound. This concentration was then used in a competition experiment to determine the Ki (binding affinity) of the compounds described in the Table below with respect to HuSA. The lower the Ki value, the more affinity a compound has for HuSA. This example shows that lipophilic conjugate groups show enhanced binding affinity to Albumin compared to an unconjugated parent.

TABLE 38
Conjugated Oligomeric Compounds Targeted to Malat-1

| Compound | | | SEQ ID |
|---|---|---|---|
| No. | Sequence (5′ to 3′) | Conjugate | NO. |
| 863776 | C16-HA-T_{do}^{m}C_{do}A_{do}Gₖₛ^{m}CₖₛAₖₛT_{ds}T_{ds}^{m}C_{ds}T_{ds}A_{ds}A_{ds}T_{ds}A_{ds} | 5′-C16-HA; 3′- | |
| | G_{ds}^{m}C_{ds}AₖₛGₖₛ^{m}Ck-Alexa Fluor | Fluorophore | |
| 556089 | Gₖₛ^{m}CₖₛAₖₛT_{ds}T_{ds}^{m}C_{ds}T_{ds}A_{ds}A_{ds}T_{ds}A_{ds}G_{ds}^{m}C_{ds}AₖₛGₖₛ^{m}Cₖ | None | 3 |
| 929856 | C8-HA- Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | C8-HA | 3 |
| | AₖₛGₖₛ^{m}Cₖ | | |
| 929847 | C10-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | C10-HA | 3 |
| | AₖₛGₖₛ^{m}Cₖ | | |
| 929853 | C12-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | C12-HA | 3 |
| | AₖₛGₖₛ^{m}Cₖ | | |
| 929854 | C14-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | C14-HA | 3 |
| | AₖₛGₖₛ^{m}Cₖ | | |
| 859299 | C16-HA-Gₖₛ^{m}CₖₛAₖₛT_{ds}T_{ds}nC_{ds}T_{ds}A_{ds}A_{ds}T_{ds}A_{ds}G_{ds}^{m}C_{ds} | C16-HA | 3 |
| | AₖₛGₖₛ^{m}Cₖ | | |
| 929855 | C18-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | C18-HA | 3 |
| | AₖₛGₖₛ^{m}Cₖ | | |
| 929857 | C20-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | C20-HA | 3 |
| | AₖₛGₖₛ^{m}Cₖ | | |
| 929858 | C22-HA-Gₖₛ^{m}CₖₛAₖₛT_{ds}T_{ds}^{m}C_{ds}T_{ds}A_{ds}A_{ds}T_{ds}A_{ds}G_{ds}^{m}C_{ds} | C22-HA | 3 |
| | AₖₛGₖₛ^{m}Cₖ | | |
| 929840 | C22-HA-T_{do}^{m}C_{do}A_{do}Gₖₛ^{m}CₖₛAₖₛT_{ds}T_{ds}^{m}C_{ds}T_{ds}A_{ds}A_{ds}T_{ds}A_{ds} | C22-HA | 4 |
| | G_{ds}^{m}C_{ds}AₖₛGₖₛ^{m}Cₖ | | |
| 950439 | Palmitoleoyl-HA-Gₖₛ^{m}CₖₛAₖₛT_{ds}T_{ds}^{m}C_{ds}T_{ds}A_{ds}A_{ds}T_{ds}A_{ds} | C16, ω 7 | 3 |
| | G_{ds}^{m}C_{ds}AₖₛGₖₛ^{m}Cₖ | (Palmitoleoyl- | |
| | | HA-) | |
| 950437 | Linoleoyl-HA-Gₖₛ^{m}CₖₛAₖₛT_{ds}T_{ds}^{m}C_{ds}T_{ds}A_{ds}A_{ds}T_{ds}A_{ds} | C18, ω 6, 9 | 3 |
| | G_{ds}^{m}C_{ds}AₖₛGₖₛ^{m}Cₖ | (Linoleoyl-HA-) | |
| 950641 | Linolenyl-HA-Gₖₛ^{m}CₖₛAₖₛT_{ds}T_{ds}^{m}C_{ds}T_{ds}A_{ds}A_{ds}T_{ds}A_{ds} | C18, ω 3, 6, 9 | 3 |
| | G_{ds}^{m}C_{ds}AₖₛGₖₛ^{m}Cₖ | (Linolenyl-HA-) | |
| 950438 | Arachidonyl-HA-Gₖₛ^{m}CₖₛAₖₛT_{ds}T_{ds}^{m}C_{ds}T_{ds}A_{ds}A_{ds}T_{ds}A_{ds}G_{ds} | C20, ω 6, 9, 12, | 3 |
| | ^{m}C_{ds}AₖₛGₖₛ^{m}Cₖ | 15 | |
| | | (Arachidonyl- | |
| | | HA-) | |
| 867593 | DHA6 ω -HA- | C22, ω 3, 6, 9, | 2 |
| | T_{do}^{m}C_{do}A_{do}Gₖₛ^{m}CₖₛAₖₛT_{ds}T_{ds}^{m}C_{ds}T_{ds}A_{ds}A_{ds}T_{ds} | 12, 15, 18 | |
| | A_{ds}G_{ds}^{m}C_{ds}AₖₛGₖₛ^{m}Cₖ | (DHA 6ω -HA-) | |
| 950642 | Elaidoyl-HA-Gₖₛ^{m}CₖₛAₖₛT_{ds}T_{ds}^{m}C_{ds}T_{ds}A_{ds}A_{ds}T_{ds}A_{ds} | C18, ω 9 | 3 |
| | G_{ds}^{m}C_{ds}AₖₛGₖₛ^{m}Cₖ | (Elaidoyl-HA-) | |
| 950643 | Linoelaidoyl-HA-Gₖₛ^{m}CₖₛAₖₛT_{ds}T_{ds}^{m}C_{ds}T_{ds}A_{ds}A_{ds}T_{ds}A_{ds}G_{ds} | C18, ω 6, 9 | 3 |
| | ^{m}C_{ds}AₖₛGₖₛ^{m}Cₖ | (Linoelaidoyl- | |
| | | HA-) | |

TABLE 39
Ki values of Conjugated Oligomeric
Compounds Targeted to Malat-1

| | |
|---|---|
| Compound | Serum Protein (Ki μM) |

| No. | Conjugate | Albumin |
|---|---|---|
| 556089 | None | 24.00 |
| 929856 | C8-HA | 2.20 |
| 929847 | C10-HA | 4.99 |
| 929853 | C12-HA | 3.22 |
| 929854 | C14-HA | 1.97 |
| 859299 | C16-HA | 0.92 |
| 929855 | C18-HA | 0.85 |
| 929857 | C20-HA | 0.91 |
| 929858 | C22-HA | 0.97 |
| 929840 | C22-HA | 0.89 |
| 950439 | C16, ω 7 | 1.70 |
| | (Palmitoleoyl-HA-) | |
| 950437 | C18, ω 6, 9 | 1.86 |
| | (Linoleoyl-HA-) | |
| 950641 | C18, ω 3, 6, 9 | 2.02 |
| | (Linolenyl-HA-) | |
| 950438 | C20, ω 6, 9, 12, 15 | 1.76 |
| | (Arachidonyl-HA-) | |
| 867593 | C22, ω 3, 6, 9, 12, 15, 18 | 5.08 |
| | (DHA 6ω -HA-) | |
| 950642 | C18, ω 9 | 1.08 |
| | (Elaidoyl-HA-) | |
| 950643 | C18, ω 6, 9 | 1.26 |
| | (Linoelaidoyl-HA-) | |

1.-238. (canceled)

239. A method of treating a disease associated with a heart target comprising administering to an individual having or at risk for developing a disease associated with the heart target a therapeutically effective amount of a pharmaceutical composition comprising an antisense compound and a pharmaceutically acceptable carrier or diluent, wherein
the antisense compound is a oligomeric compound comprising a modified oligonucleotide and a conjugate group,
wherein the modified oligonucleotide consist of 10-30 linked nucleosides and has a nucleobase sequence complementary to the nucleobase sequence of a heart target,
wherein the conjugate group comprises a conjugate moiety and a conjugate linker, wherein the conjugate moiety is selected from C₁₀-C₂₆ saturated fatty acid, C₁₀-C₂₆ unsaturated fatty acid, C₁₀-C₂₆ alkyl, or C₁₀-C₂₆ alkenyl.

240. The method of claim 239, wherein the conjugate group consists of the conjugate moiety and conjugate linker.

241. The method of claim 239, wherein the oligomeric compound is single-stranded.

242. The method of claim 239, wherein the oligomeric compound is not an RNAi compound.

243. The method of claim 239, wherein the modified oligonucleotide comprises at least one modified nucleoside comprising a modified sugar moiety.

244. The method of claim 243, wherein the modified oligonucleotide comprises at least one modified nucleoside comprising a bicyclic sugar moiety having a 2′-4′ bridge.

245. The method of claim 243, wherein the modified oligonucleotide comprises at least one modified nucleoside comprising a non-bicyclic sugar moiety comprising a 2′-MOE or 2′-OMe.

246. The method of claim 243, wherein the modified oligonucleotide has a sugar motif comprising:
a 5′-region consisting of 1-5 linked 5′-nucleosides;
a central region consisting of 6-10 linked central region nucleosides; and
a 3′-region consisting of 1-5 linked 3′-region nucleosides; wherein
each of the 5′-region nucleosides and each of the 3′-region comprises a modified sugar moiety.

247. The method of claim 243, wherein the modified oligonucleotide has a sugar motif comprising:
a 5′-region consisting of 5 linked 5′-nucleosides;
a central region consisting of 10 linked central region nucleosides; and
a 3′-region consisting of 5 linked 3′-region nucleosides; wherein
each of the 5′-region nucleosides and each of the 3′-region comprises a modified sugar moiety and each of the central region nucleosides comprises an unmodified DNA sugar moiety.

248. The method of claim 243, wherein the modified oligonucleotide has a sugar motif comprising:
a 5′-region consisting of 3 linked 5′-nucleosides;
a central region consisting of 10 linked central region nucleosides; and
a 3′-region consisting of 3 linked 3′-region nucleosides; wherein
each of the 5′-region nucleosides and each of the 3′-region comprises a modified sugar moiety and each of the central region nucleosides comprises an unmodified DNA sugar moiety.

249. The method of claim 243, wherein the modified oligonucleotide is a gapmer.

250. The method of claim 239, wherein the modified oligonucleotide comprises at least one modified internucleoside linkage.

251. The method of claim 250, wherein the at least one modified internucleoside linkage is a phosphorothioate internucleoside linkage.

252. The method of claim 250, wherein each internucleoside linkage of the modified oligonucleotide is selected from a phosphodiester internucleoside linkage and a phosphorothioate internucleoside linkage.

253. The method of claim 250, wherein the modified oligonucleotide consists of 12-20 linked nucleosides.

254. The method of claim 239, wherein the conjugate moiety is selected from palmitoyl, elaidoyl, linoelaidoyl, palmitoleoyl, linoleoyl, linolenyl, arachidonyl, erucoyl, sapienoyl, myristolenoyl, (E)-11-octadecenoyl, γ-linolenoyl, nervonoyl, and docosahexaenoic acid.

255. The method of claim 239, wherein the conjugate linker comprises 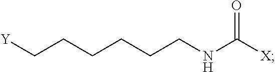
X directly or indirectly attaches to the conjugate moiety; and
Y comprises a phosphate group that directly attaches to the modified oligonucleotide.

256. The method of claim 239, wherein the conjugate group is attached to the 5′ end of the modified oligonucleotide.

257. The method of claim 239, wherein the pharmaceutical composition is administered subcutaneously.

258. The method of claim 239, wherein the pharmaceutical composition is administered intravenously.
